SEQUENCE LISTING (111 sequences)

SEQ ID NO: 1 - protein, 119 aa, Unknown
  Wild type polypeptide
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPS

SEQ ID NO: 2 - protein, 119 aa, Artificial Sequence
  Synthetic Construct
  VARIANT 6: Xaa = Met, Ile, Leu or Phe
  VARIANT 27: Xaa = Phe, Ile, Leu or Val
  VARIANT 30: Xaa = Leu, Ile, Val, His, Asn or Asp
  VARIANT 31: Xaa = Phe, Ile, Leu or Val
  VARIANT 33: Xaa = Val, Ile, Leu, Pro, Thr or Ala
  VARIANT 36: Xaa = Val or Ile
  VARIANT 37: Xaa = Leu or Gln
  VARIANT 42: Xaa = Val or Ala
  VARIANT 47: Xaa = Glu or Val
  VARIANT 52: Xaa = Gln, Pro, Leu, Val, Ala or Glu
  VARIANT 53: Xaa = Lys or Arg
  VARIANT 54: Xaa = Glu, Asp, Lys, Asn, Gln or His
  VARIANT 56: Xaa = His, Pro or Arg
  VARIANT 66: Xaa = Leu, Ile, Val, Pro, Thr, Ala, Arg, Ser or Gly
  VARIANT 67: Xaa = Thr, Ile, Asn, Phe, Ser, Tyr, Val, Ala or Asp
  VARIANT 92: Xaa = Val or Ile
  VARIANT 98: Xaa = Ser, Arg, Asn, Lys, Thr, Ile or Met
  VARIANT 101: Xaa = Asn, Lys, Asp, Glu, His or Gln
EEELQXIQPEKLLLVTVGKTATLHCTXTSXXPXGPXXWFRGXGPGRXLIYNXXXGXFPRVTTVSDXXKRNNMDFSIRISSITPADVGTYYCXKFRKGXPEXVEFKSGPGTEMALGAKPS

SEQ ID NO: 3 - protein, 119 aa, Artificial Sequence
  Synthetic Construct
EEELQIIQPEKLLLVTVGKTATLHCTITSLFPVGPVLWFRGVGPGRVLIYNQRQGPFPRVTTVSDTTKRNNMDFSIRISSITPADVGTYYCIKFRKGSPENVEFKSGPGTEMALGAKPS

SEQ ID NO: 4 - protein, 119 aa, Artificial Sequence
  Synthetic Construct
EEELQIIQPEKLLLVTVGKTATLHCTITSLFPVGPIQWFRGVGPGRVLIYNQRDGPFPRVTTVSDGTKRNNMDFSIRISSITPADVGTYYCVKFRKGTPEDVEFKSGPGTEMALGAKPS

SEQ ID NO: 5 - protein, 119 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPS

SEQ ID NO: 6 - protein, 119 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPS

SEQ ID NO: 7 - protein, 119 aa, Artificial Sequence
  Synthetic Construct
EEELQIIQPEKLLLVTVGKTATLHCTITSLFPVGPIQWFRGAGPGRVLIYNQRDGPFPRVTTVSDGTKRNNMDFSIRISSITPADVGTYYCIKFRKGTPEDVEFKSGPGTEMALGAKPS

SEQ ID NO: 8 - protein, 119 aa, Artificial Sequence
  Synthetic Construct
EEELQIIQPEKLLLVTVGKTATLHCTITSHFPVGPIQWFRGVGPGRVLIYNQKDGHFPRVTTVSDGTKRNNMDFSIRISSITPADVGTYYCVKFRKGSPEDVEFKSGPGTEMALGAKPS

SEQ ID NO: 9 - protein, 119 aa, Artificial Sequence
  Synthetic Construct
EEELQIIQPDKSVLVAAGETATLRCTITSLFPVGPIQWFRGAGPGRVLIYNQRDGPFPRVTTVSDGTKRNNMDFSIRISSITPADVGTYYCVKFRKGTPEDVEFKSGPGTEMALGAKPS

SEQ ID NO: 10 - protein, 119 aa, Artificial Sequence
  Synthetic Construct
EEELQIIQPEKLLLVTVGKTATLHCTITSLFPVGPVLWFRGVGPGRVLIYNQRQGPFPRVTTVSDTTKRNNMDFSIRISSITPADVGTYYCVKFRKGTPEDVEFKSGPGTEMALGAKPS

SEQ ID NO: 11 - protein, 119 aa, Artificial Sequence
  Synthetic Construct
EEELQIIQPEKLLLVTVGKTATLHCTITSLFPVGPIQWFRGVGPGRELIYNAREGRFPRVTTVSDLTKRNNMDFSIRISSITPADVGTYYCVKFRKGSPEDVEFKSGPGTEMALGAKPS

SEQ ID NO: 12 - protein, 119 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 13 - protein, 119 aa, Artificial Sequence
  Synthetic Construct
EEELQIIQPEKLLLVTVGKTATLHCTITSLFPVGPIQWFRGVGPGRVLIYNQREGPFPRVTTVSDGTKRNNMDFSIRISSITPADVGTYYCVKFRKGSPEDVEFKSGPGTEMALGAKPS

SEQ ID NO: 14 - protein, 119 aa, Artificial Sequence
  Synthetic Construct
EEELQIIQPDKSVLVAAGETATLRCTITSLFPVGPIQWFRGAGPGRVLIYNQRDGPFPRVTTVSDGTKRNNMDFSIRISSITPADVGTYYCIKFRKGIPEDVEFKSGPGTEMALGAKPS

SEQ ID NO: 15 - protein, 113 aa, Artificial Sequence
  Synthetic Construct
  VARIANT 111: Xaa = Any Amino Acid
EEELQIIQPDKSVLVAAGETATLRCTITSLFPVGPIQWFRGAGPGRVLIYNQRDGPFPRVTTVSDGTKRNNMDFSIRISSITPADVGTYYCIKFRKGIPEDVEFKSGPGTXWH

SEQ ID NO: 16 - protein, 119 aa, Artificial Sequence
  Synthetic Construct
EEELQIIQPDKSVLVAAGETATLRCTITSLFPVGPIQWFRGAGPGRVLIYNQKDGPFPRVTTVSDGTKRNNMDFSIRISSITPADVGTYYCIKFRKGTPEDVEFKSGPGTEMALGAKPS

SEQ ID NO: 17 - protein, 119 aa, Artificial Sequence
  Synthetic Construct
EEELQIIQPEKLLLVTVGKTATLHCTITSLLPVGPIQWFRGVGPGRELIYNQRDGPFPRVTTVSDGTKRNNMDFSIRISSITPADVGTYYCVKFRKGTPEDVEFKSGPGTEMALGAKPS

SEQ ID NO: 18 - protein, 119 aa, Artificial Sequence
  Synthetic Construct
EEELQIIQPEKLLLVTVGKTATLHCTLTSLLPVGPILWFRGVGPGRVLIYNQRDGPFPRVTTVSDGTKRNNMDFSIRISSITPADVGTYYCVKFRKGNPEDVEFKSGPGTEMALGAKPS

SEQ ID NO: 19 - protein, 119 aa, Artificial Sequence
  Synthetic Construct
EEELQLIQPEKLLLVTVGKTATLHCTITSLFPPGPIQWFRGVGPGRVLIYNQKDGPFPRVTTVSDGTKRNNMDFSIRISSITPADVGTYYCVKFRKGIPEDVEFKSGPGTEMALGAKPS

SEQ ID NO: 20 - protein, 119 aa, Artificial Sequence
  Synthetic Construct
EEELQIIQPEKLLLVTVGKTATLRCTITSLFPVGPIQWFRGAGPGRVLIYNQRDGPFPRVTTVSDGTKRNNMDFSIRISSITPADVGTYYCIKFRKGIPEDVEFKSGPGTEMALGAKPS

SEQ ID NO: 21 - protein, 119 aa, Artificial Sequence
  Synthetic Construct
EEELQIIQPEKLLLVTVGKTATLHCTITSLFPIGPILWFRGVGPGRVLIYNQKDGPFPRVTTVSDGTKRNNMDFSIRISSITPADVGTYYCVKFRKGSPEDVEFKSGPGTEMALGAKPS

SEQ ID NO: 22 - protein, 119 aa, Artificial Sequence
  Synthetic Construct
EEELQMIQPEKLLLVTVGKTATLHCTITSLFPVGPIQWFRGAGPGRVLIYNQRDGPFPRVTTVSDGTKRNNMDFSIRISSITPADVGTYYCIKFRKGIPEDVEFKSGPGTEMALGAKPS

SEQ ID NO: 23 - protein, 119 aa, Artificial Sequence
  Synthetic Construct
EEELQIIQPDKSVLVAAGETATLRCTITSLFPVGPIQWFRGVGPGRVLIYNQKDGPFPRVTTVSDGTKRNNMDFSIRISSITPADVGTYYCIKFRKGIPEDVEFKSGPGTEMALGAKPS

SEQ ID NO: 24 - protein, 119 aa, Artificial Sequence
  Synthetic Construct
  VARIANT 115: Xaa = Any Amino Acid
EEELQIIQPDKSVLVAAGETATLRCTITSLFPVGPIQWFRGAGPGRVLIYNQKDGPFPRVTTVSDGTKRNNMDFSIRISSITPADVGTYYCIKFRKGTPEDVEFKSGPGTEMALXAKPS

SEQ ID NO: 25 - protein, 119 aa, Artificial Sequence
  Synthetic Construct
EDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPS

SEQ ID NO: 26 - protein, 119 aa, Artificial Sequence
  Synthetic Construct
EDELQIIQPEKSVSVAAGESATLRCAITSLFPVGPIQWFRGAGAGRVLIYNQRQGPFPRVTTVSETTKRNNLDFSISISNITPADAGTYYCIKFRKGSPDDVEFKSGAGTELSVRAKPS

SEQ ID NO: 27 - protein, 119 aa, Artificial Sequence
  Synthetic Construct
EEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDHVEFKSGAGTELSVRAKPS

SEQ ID NO: 28 - protein, 119 aa, Artificial Sequence
  Synthetic Construct
EEELQIIQPDKSISVAAGESATLHCTITSLFPVGPIQWFRGAGPGRVLIYNQRQGPFPRVTTVSDTTKRNNMDFSIRISNITPADAGTYYCIKFRKGSPDDVEFKSGAGTELSVRAKPS

SEQ ID NO: 29 - protein, 10 aa, Artificial Sequence
  Synthetic Construct
GGGGSGGGGS

SEQ ID NO: 30 - protein, 251 aa, Artificial Sequence
  Synthetic Construct
DSPDRPWNPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTERGGGGSGGGGSEEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPS

SEQ ID NO: 31 - protein, 241 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVK

SEQ ID NO: 32 - protein, 291 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 33 - protein, 243 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTT

SEQ ID NO: 34 - protein, 245 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPA

SEQ ID NO: 35 - protein, 263 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTDEKFNLKLVIKPAKVTPA

SEQ ID NO: 36 - protein, 275 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 37 - protein, 296 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSDPAGLLDLRQGMFAQLVAQNVLLIDGPLSWYSDPGLAGVSLTGGLSYKEDTKELVVAKAGVYYVFFQMELRRVVAGEGSGSVSLALHLMPLRSAAGAAALALTVDLPPASSEARNSAFGFQGRLLHLSAGQRLGVHLHTEARARHAWQLTQGATVLGLFRVTPEIPA

SEQ ID NO: 38 - protein, 262 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 39 - protein, 262 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSGGGGSGGGGSAPTSSSTKKTQLQLEHLLLTLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 40 - protein, 8 aa, Artificial Sequence
  Synthetic 8xHis tag
HHHHHHHH

SEQ ID NO: 41 - protein, 15 aa, Artificial Sequence
  Synthetic Construct
GLNDIFEAQKIEWHE

SEQ ID NO: 42 - protein, 119 aa, Artificial Sequence; identical to SEQ ID NO: 1
  Synthetic Construct

SEQ ID NO: 43 - protein, 119 aa, Unknown; identical to SEQ ID NO: 25
  Wild type polypeptide

SEQ ID NO: 44 - protein, 119 aa, Unknown; identical to SEQ ID NO: 27
  Wild type polypeptide

SEQ ID NO: 45 - protein, 119 aa, Artificial Sequence
  Synthetic Construct
  VARIANT 6: Xaa = Val or Ile
  VARIANT 27: Xaa = Met or Ile
  VARIANT 31: Xaa = Ile or Phe
  VARIANT 37: Xaa = Met or Gln
  VARIANT 47: Xaa = Glu or Val
  VARIANT 53: Xaa = Lys or Arg
  VARIANT 54: Xaa = Glu or Gln
  VARIANT 56: Xaa = His or Pro
  VARIANT 66: Xaa = Leu or Thr
  VARIANT 92: Xaa = Val or Ile
EDELQXIQPEKSVSVAAGESATLRCAXTSLXPVGPIXWFRGAGAGRXLIYNQXXGXFPRVTTVSEXTKRNNLDFSISISNITPADAGTYYCXKFRKGSPDDVEFKSGAGTELSVRAKPS

SEQ ID NO: 46 - protein, 119 aa, Artificial Sequence
  Synthetic Construct
  VARIANT 6: Xaa = Val or Ile
  VARIANT 27: Xaa = Val or Ile
  VARIANT 31: Xaa = Ile or Phe
  VARIANT 47: Xaa = Glu or Val
  VARIANT 53: Xaa = Lys or Arg
  VARIANT 54: Xaa = Glu or Gln
  VARIANT 56: Xaa = His or Pro
  VARIANT 66: Xaa = Leu or Thr
  VARIANT 92: Xaa = Val or Ile
  VARIANT 101: Xaa = His or Asp
EEELQXIQPDKSISVAAGESATLHCTXTSLXPVGPIQWFRGAGPGRXLIYNQXXGXFPRVTTVSDXTKRNNMDFSIRISNITPADAGTYYCXKFRKGSPDXVEFKSGAGTELSVRAKPS

SEQ ID NO: 47 - protein, 226 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 226 aa, Artificial Sequence
  Synthetic Construct
DKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 226 aa, Artificial Sequence
  Synthetic Construct
DKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 226 aa, Artificial Sequence
  Synthetic Construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 226 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 222 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 53 - protein, 222 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 54 - protein, 222 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 55 - protein, 228 aa, Artificial Sequence
  Synthetic Construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 228 aa, Artificial Sequence
  Synthetic Construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 345 aa, Artificial Sequence
  Synthetic Construct
EEELQIIQPEKLLLVTVGKTATLHCTITSLFPVGPVLWFRGVGPGRVLIYNQRQGPFPRVTTVSDTTKRNNMDFSIRISSITPADVGTYYCIKFRKGSPENVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 345 aa, Artificial Sequence
  Synthetic Construct
EEELQIIQPEKLLLVTVGKTATLHCTITSLFPVGPIQWFRGVGPGRVLIYNQRDGPFPRVTTVSDGTKRNNMDFSIRISSITPADVGTYYCVKFRKGTPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 345 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 345 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 345 aa, Artificial Sequence
  Synthetic Construct
EEELQIIQPEKLLLVTVGKTATLHCTITSLFPVGPIQWFRGAGPGRVLIYNQRDGPFPRVTTVSDGTKRNNMDFSIRISSITPADVGTYYCIKFRKGTPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 345 aa, Artificial Sequence
  Synthetic Construct
EEELQIIQPEKLLLVTVGKTATLHCTITSHFPVGPIQWFRGVGPGRVLIYNQKDGHFPRVTTVSDGTKRNNMDFSIRISSITPADVGTYYCVKFRKGSPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 345 aa, Artificial Sequence
  Synthetic Construct
EEELQIIQPEKLLLVTVGKTATLHCTITSLFPVGPVLWFRGVGPGRVLIYNQRQGPFPRVTTVSDTTKRNNMDFSIRISSITPADVGTYYCVKFRKGTPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 345 aa, Artificial Sequence
  Synthetic Construct
EEELQIIQPEKLLLVTVGKTATLHCTITSLFPVGPIQWFRGVGPGRELIYNAREGRFPRVTTVSDLTKRNNMDFSIRISSITPADVGTYYCVKFRKGSPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 345 aa, Artificial Sequence
  Synthetic Construct
EEELQIIQPEKLLLVTVGKTATLHCTITSLFPVGPIQWFRGVGPGRVLIYNQREGPFPRVTTVSDGTKRNNMDFSIRISSITPADVGTYYCVKFRKGSPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 345 aa, Artificial Sequence
  Synthetic Construct
EEELQIIQPEKLLLVTVGKTATLHCTITSLLPVGPIQWFRGVGPGRELIYNQRDGPFPRVTTVSDGTKRNNMDFSIRISSITPADVGTYYCVKFRKGTPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 345 aa, Artificial Sequence
  Synthetic Construct
EEELQIIQPEKLLLVTVGKTATLHCTLTSLLPVGPILWFRGVGPGRVLIYNQRDGPFPRVTTVSDGTKRNNMDFSIRISSITPADVGTYYCVKFRKGNPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 345 aa, Artificial Sequence
  Synthetic Construct
EEELQLIQPEKLLLVTVGKTATLHCTITSLFPPGPIQWFRGVGPGRVLIYNQKDGPFPRVTTVSDGTKRNNMDFSIRISSITPADVGTYYCVKFRKGIPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 345 aa, Artificial Sequence
  Synthetic Construct
EEELQIIQPEKLLLVTVGKTATLHCTITSLFPIGPILWFRGVGPGRVLIYNQKDGPFPRVTTVSDGTKRNNMDFSIRISSITPADVGTYYCVKFRKGSPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 345 aa, Artificial Sequence
  Synthetic Construct
EEELQMIQPEKLLLVTVGKTATLHCTITSLFPVGPIQWFRGAGPGRVLIYNQRDGPFPRVTTVSDGTKRNNMDFSIRISSITPADVGTYYCIKFRKGIPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 345 aa, Artificial Sequence
  Synthetic Construct
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 345 aa, Artificial Sequence
  Synthetic Construct
EDELQIIQPEKSVSVAAGESATLRCAITSLFPVGPIQWFRGAGAGRVLIYNQRQGPFPRVTTVSETTKRNNLDFSISISNITPADAGTYYCIKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 345 aa, Artificial Sequence
  Synthetic Construct
EEELQIIQPDKSISVAAGESATLHCTITSLFPVGPIQWFRGAGPGRVLIYNQRQGPFPRVTTVSDTTKRNNMDFSIRISNITPADAGTYYCIKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 345 aa, Artificial Sequence; identical to SEQ ID NO: 71
  Synthetic Construct

SEQ ID NO: 75 - protein, 345 aa, Artificial Sequence
  Synthetic Construct
EDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 345 aa, Artificial Sequence
  Synthetic Construct
EEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDHVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 345 aa, Artificial Sequence
  Synthetic Construct
EEELQIIQPDKSVLVAAGETATLRCTITSLFPVGPIQWFRGAGPGRELIYNQREGPFPRVTTVSDTTKRNNMDFSIRIGAITPADAGTYYCVKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 119 aa, Artificial Sequence
  Synthetic Construct
EEELQIIQPDKSVLVAAGETATLRCTITSLFPVGPIQWFRGAGPGRELIYNQREGPFPRVTTVSDTTKRNNMDFSIRIGAITPADAGTYYCVKFRKGSPDDVEFKSGAGTELSVRAKPS

SEQ ID NO: 79 - protein, 226 aa, Artificial Sequence; identical to SEQ ID NO: 49
  Synthetic Construct

SEQ ID NO: 80 - protein, 117 aa, Artificial Sequence
  Synthetic Construct
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVS

SEQ ID NO: 81 - protein, 119 aa, Artificial Sequence
  Synthetic Construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 82 - protein, 345 aa, Artificial Sequence
  Synthetic Construct
EEELQIIQPDKSVLVAAGETATLRCTITSLFPVGPIQWFRGAGPGRVLIYNQRDGPFPRVTTVSDGTKRNNMDFSIRISSITPADVGTYYCVKFRKGTPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 345 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 345 aa, Artificial Sequence
  Synthetic Construct
EEELQIIQPDKSVLVAAGETATLRCTITSLFPVGPIQWFRGAGPGRVLIYNQRDGPFPRVTTVSDGTKRNNMDFSIRISSITPADVGTYYCIKFRKGIPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 345 aa, Artificial Sequence
  Synthetic Construct
EEELQIIQPDKSVLVAAGETATLRCTITSLFPVGPIQWFRGAGPGRVLIYNQKDGPFPRVTTVSDGTKRNNMDFSIRISSITPADVGTYYCIKFRKGTPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 345 aa, Artificial Sequence
  Synthetic Construct
EEELQIIQPEKLLLVTVGKTATLRCTITSLFPVGPIQWFRGAGPGRVLIYNQRDGPFPRVTTVSDGTKRNNMDFSIRISSITPADVGTYYCIKFRKGIPEDVEFKSGPGTEMALGAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 345 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 119 aa, Artificial Sequence
  Synthetic Construct
EDELQIIQPEKSVSVAAGESATLRCAITSLFPVGPIQWFRGAGAGRVLIYNQRQGPFPRVTTVSETTKRNNLDFSISISAITPADAGTYYCIKFRKGSPDDVEFKSGAGTELSVRAKPS

SEQ ID NO: 89 - protein, 119 aa, Artificial Sequence
  Synthetic Construct
EEELQIIQPDKSISVAAGESATLHCTITSLFPVGPIQWFRGAGPGRVLIYNQRQGPFPRVTTVSDTTKRNNMDFSIRISAITPADAGTYYCIKFRKGSPDDVEFKSGAGTELSVRAKPS

SEQ ID NO: 90 - protein, 345 aa, Artificial Sequence
  Synthetic Construct
EDELQIIQPEKSVSVAAGESATLRCAITSLFPVGPIQWFRGAGAGRVLIYNQRQGPFPRVTTVSETTKRNNLDFSISISAITPADAGTYYCIKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 345 aa, Artificial Sequence
  Synthetic Construct
EEELQIIQPDKSISVAAGESATLHCTITSLFPVGPIQWFRGAGPGRVLIYNQRQGPFPRVTTVSDTTKRNNMDFSIRISAITPADAGTYYCIKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 226 aa, Artificial Sequence; identical to SEQ ID NO: 47
  Synthetic Construct

SEQ ID NO: 93 - protein, 226 aa, Artificial Sequence; identical to SEQ ID NO: 48
  Synthetic Construct

SEQ ID NO: 94 - protein, 227 aa, Artificial Sequence
  Synthetic Construct
DKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 227 aa, Artificial Sequence
  Synthetic Construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 227 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 223 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 98 - protein, 223 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 99 - protein, 223 aa, Artificial Sequence
  Synthetic Construct
VECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 229 aa, Artificial Sequence
  Synthetic Construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 229 aa, Artificial Sequence
  Synthetic Construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 250 aa, Artificial Sequence
  Synthetic Construct
DSPDRPWNPPTFSPALLVVTEGDNATFTCSFSNTSESFHVVWHRESPSGQTDTLAFPEDRSQPGQDARFRVTQLPNGRDFHMSVVRARRNDSGTYVCGVISLAPKIQIKESLRAELRVTERGGGGSGGGGSEEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPS

SEQ ID NO: 103 - protein, 241 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVK

SEQ ID NO: 104 - protein, 291 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 105 - protein, 243 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTT

SEQ ID NO: 106 - protein, 245 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPA

SEQ ID NO: 107 - protein, 263 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTDEKFNLKLVIKPAKVTPA

SEQ ID NO: 108 - protein, 275 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 109 - protein, 296 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSDPAGLLDLRQGMFAQLVAQNVLLIDGPLSWYSDPGLAGVSLTGGLSYKEDTKELVVAKAGVYYVFFQMELRRVVAGEGSGSVSLALHLMPLRSAAGAAALALTVDLPPASSEARNSAFGFQGRLLHLSAGQRLGVHLHTEARARHAWQLTQGATVLGLFRVTPEIPA

SEQ ID NO: 110 - protein, 262 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 111 - protein, 262 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPSGGGGSGGGGSAPTSSSTKKTQLQLEHLLLTLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2021/0347848 A1
(43) Pub. Date: Nov. 11, 2021

(54) DECOY POLYPEPTIDES

(71) Applicant: ALX Oncology Inc., Burlingame, CA (US)

(72) Inventors: Bang Janet SIM, Brisbane, CA (US); Jaume PONS, San Francisco, CA (US)

(21) Appl. No.: 17/270,683

(22) PCT Filed: Aug. 29, 2019
(86) PCT No.: PCT/US2019/048921

§ 371 Date: Feb. 23, 2021

Related U.S. Application Data
(60) Provisional application No. 62/725,977, filed on Aug. 31, 2018.

(51) Int. Cl. C07K 14/705; C07K 14/55; C07K 16/18; A61K 45/06

(52) U.S. Cl. CPC C07K 14/70503; C07K 14/55; C07K 16/18; A61K 45/06; A61K 38/00; C07K 2319/30; C07K 2317/71; C07K 2317/40; C07K 2317/92; C07K 2319/32

(57) ABSTRACT

Provided are decoy polypeptides comprising: (a) a SIRPγ variant, a SIRPβ1 variant, or a SIRPβ2 variant, and (b) a human Fc variant comprising at least one amino acid substitution that reduces effector function compared to a wild type human Fc. Also provided are methods of using such decoy polypeptides, and chimeric molecules comprising such polypeptides.

### CROSS REFERENCE TO RELATED APPLICATIONS

This application claims the priority benefit of U.S. Provisional Application Ser. No. 62/725,977, filed Aug. 31, 2018, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 757972000740SEQLIST.txt, date recorded: Aug. 29, 2019, size: 212 KB).

### BACKGROUND OF THE INVENTION

Signal regulatory proteins (SIRPs) constitute a family of cell surface glycoproteins which are expressed on myeloid cells (including macrophages, granulocytes, myeloid dendritic cells, and mast cells), lymphocytes, and neuronal cells and regulate their activity.

### SUMMARY OF THE INVENTION

Provided herein is a decoy polypeptide comprising: (a) a SIRPγ variant and (b) a human Fc variant comprising at least one amino acid substitution that ablates effector function or reduces effector function compared to a wild type human Fc, wherein the SIRPγ variant comprises at least one amino acid substitution relative to a wild type SIRPγ, which substitution increases the affinity of the SIRPγ variant for CD47 as compared to the affinity of the wild type SIRPγ for CD47, and wherein the SIRPγ variant lacks a transmembrane domain. In some embodiments, the at least one amino acid substitution is within a d1 domain of the SIRPγ variant. In some embodiments, the amino acid sequence of the d1 domain of the SIRPγ variant is at least 90% identical to a sequence of a wild type SIRPγ d1 domain set forth in EEELQMIQPEKLLLVTVGKTATLHCTVTSLLPVGPVLWFRGVGPGRELIYNQKEGHF PRVTTVSDLTKRN NMDFSIRISS ITPADVGTYY CVKFRKGSPENVEFKSGPGTEMALGAKPS (SEQ ID NO: 1). In some embodiments, the SIRPγ variant comprises one or more amino acid substitutions at M6, V27, L30, L31, V33, V36, L37, V42, E47, Q52, K53, E54, H56, L66, T67, V92, S98 or N101, wherein the amino acid positions are relative to the wild-type human SIRPγ d1 domain sequence set forth in SEQ ID NO: 1. In some embodiments, the SIRPγ variant comprises the M6 substitution, and wherein the substitution is M6I, M6L or M6F. In some embodiments, the γ variant comprises the V27 substitution, and wherein the substitution is V27F, V27I or V27L. In some embodiments, the SIRPγ variant comprises the L30 substitution, and wherein the substitution is L30I, L30V, L30H, L30N or L30D. In some embodiments, the SIRPγ variant comprises the L31 substitution, and wherein the substitution is L31F, L31I, L31V, L31T, or L31S. In some embodiments, the SIRPγ variant comprises the V33 substitution, and wherein the substitution is V33I, V33L, V33P, V33T, or V33A. In some embodiments, the SIRPγ variant comprises the V36 substitution, and wherein the substitution is V36I. In some embodiments, the SIRPγ variant comprises the L37 substitution, and wherein the substitution is L37Q. In some embodiments, the SIRPγ variant comprises the V42 substitution, and wherein the substitution is V42A. In some embodiments, the SIRPγ variant comprises the E47 substitution, and wherein the substitution is E47V. In some embodiments, the SIRPγ variant comprises the Q52 substitution, and wherein the substitution is Q52P, Q52L, Q52V, Q52A or Q52E. In some embodiments, the SIRPγ variant comprises the K53 substitution, and wherein the substitution is K53R. In some embodiments, the SIRPγ variant comprises E54 substitution, and wherein the substitution is E54D, E54K, E54N, E54Q, or E54H. In some embodiments, the SIRPγ variant comprises the H56 substitution, and wherein the substitution is H56P or H56R. In some embodiments, the SIRPγ variant comprises the L66 substitution, and wherein the substitution is L66I, L66V, L66P, L66T, L66A, L66R, L66S or L66G. In some embodiments, the SIRPγ variant comprises the T67 substitution, and wherein the substitution is T67I, T67N, T67F, T67S, T67Y, T67V, T67A or T67D. In some embodiments, the SIRPγ variant comprises the V92 substitution, and wherein the substitution is V92I. In some embodiments, the SIRPγ variant comprises the S98 substitution, and wherein the substitution is S98R, S98N, S98K, S98T, S981 or S98M. In some embodiments, the SIRPγ variant comprises the N101 substitution, and wherein the substitution is N101K, N101D, N101E, N101H or N101Q.

In some embodiments, the SIRPγ variant comprises an amino acid sequence set forth in EEELQX₁IQPEKLLLVTVGKTATLHCTX₂TSX₃X₄PX₅GPX₆X₇WFRGX₈GPGRX₉LIYNX₁₀X₁₁X₁₂GX₁₃FPRVTTVSDX₁₄X₁₅KRNNMDFSIRISSITPADVGTYYCX₁₆KFRKGX₁₇PEX₁₈VEFKSGPGTEMALGAKPS (SEQ ID NO: 2), wherein X₁ is M, I, L or F; X₂ is F, I, L or V; X₃ is L, I, V, H, N or D; X₄ is F, I, L, V, T, and S; X₅ is V, I, L, P, T or A; X₆ is V or I; X₇ is L or Q; X₈ is V or A; X₉ is E or V; X₁₀ is Q, P, L, V, A or E; X₁₁ is K or R; X₁₂ is E, D, K, N, Q or H; X₁₃ is H, P or R; X₁₄ is L, I, V, P, T, A, R, S or G; X₁₅ is T, I, N, F, S, Y, V, A or D; X₁₆ is V or I; X₁₇ is S, R, N, K, T, I or M; and X₁₈ is N, K, D, E, H or Q.

In some embodiments, the SIRPγ variant comprises an amino acid sequence set forth in any one of SEQ ID NOs: 3-14, 16-24, and 42. In some embodiments, the SIRPγ variant comprises an amino acid sequence set forth in EEELQIIQPDKSVLVAAGETATLRCTITSLFPVGPIQWFRGAGPGRVLIYNQRDGPFPR V TTVSDGTKRNNMDFSIRISSITPADVGTYYCIKFRKGIPEDVEFKSGPGTXWH (SEQ ID NO: 15), wherein X is A, R, N, D, C, Q, E, G, H, I, L, K, M, F, P, S, T, W, Y, or V.

In some embodiments, the decoy polypeptide comprises the amino acid sequence of any one of SEQ ID NOs: 57-71 and 82-86 or an amino acid sequence that is at least about 90%, 91%, 92%, 93%, 94%, 95%, 96%, 97% 98% or 99% identical to any one of SEQ ID NOs: 57-71, 74, and 82-86.

In a related aspect, provided herein is a decoy polypeptide comprising: (a) a SIRPβ1 variant, and (b) a human Fc variant comprising at least one amino acid substitution that reduces effector function compared to a wild type human Fc, wherein the SIRPβ1 variant comprises at least one amino acid substitution relative to a wild type SIRPβ1, which substitution increases the affinity of the SIRPβ1 variant for CD47 as compared to the affinity of the wild type SIRPβ1 for CD47, and wherein the SIRPβ1 variant lacks a transmembrane domain. In some embodiments, the at least one amino acid substitution is within a d1 domain of the SIRPβ1 variant. In some embodiments, the amino acid sequence of the d1 domain of the SIRPβ1 variant is at least 90% identical to a sequence of a wild type SIRPβ1 domain set forth in EDELQVIQPEKSVSVAAGESATLRCAMTSLIPVGPIMWFRGAGAGRELIYNQKEGHF PRVTTVSELTKRNNLDFSISISNITPADAGTYYCVKFRKGSPDDVEFKSGAGTELSVR AKPS (SEQ ID NO: 25). In some embodiments, the SIRPβ1 variant comprises one or more amino acid substitution at V6, M27, 131, M37, E47, K53, E54, H56, L66, N80, or V92, wherein the amino acid positions are relative to a wild-type human SIRPβ1 d1 domain sequence set forth in SEQ ID NO: 25. In some embodiments, the SIRPβ1 variant comprises the V6 substitution, and wherein the substitution is V6I. In some embodiments, the SIRPβ1 variant comprises the M27 substitution, and wherein the substitution is M27I. In some embodiments, the SIRPβ1 variant comprises the I31 substitution, and wherein the substitution is I31F. In some embodiments, the SIRPβ1 variant comprises the M37 substitution, and wherein the substitution is M37Q. In some embodiments, the SIRPβ1 variant comprises the E47 substitution, and wherein the substitution is E47V. In some embodiments, the SIRPβ1 variant comprises the K53 substitution, and wherein the substitution is K53R. In some embodiments, the SIRPβ1 variant comprises the E54 substitution, and wherein the substitution is E54Q. In some embodiments, the SIRPβ1 variant comprises the H56 substitution, and wherein the substitution is H56P. In some embodiments, the SIRPβ1 variant comprises the L66 substitution, and wherein the substitution is L66T. In some embodiments, the SIRPβ1 variant comprises the N80 substitution, and wherein the substitution is N80A, N80C, N80D, N80E, N80F, N80G, N80H, N80I, N80K, N80L, N80M, N80P, N80Q, N80R, N80S, N80T, N80V, N80W, or N80Y. In some embodiments, the SIRPβ1 variant comprises the V92 substitution, and wherein the substitution is V92I. In some embodiments, the SIRPβ1 variant comprises an amino acid sequence of EDELQIIQPEKSVSVAAGESATLRCAITSLFPVGPIQWFRGAGAGRVLIYNQRQGP FPRVTTVSETTKRNNLDFSISISNITPADAGTYYCIKFRKGSPDDVEFKSGAGTEL SVRAKPS (SEQ ID NO: 26). In some embodiments, the SIRPβ1 variant comprises an amino acid sequence of EDELQIIQPEKSVSVAAGESATLRCAITSLFPVGPIQWFRGAGAGRVLIYNQRQGPFPR VTTVSETTKRNNLDFSISISAITPADAGTYYCIKFRKGSPDDVEFKSGAGTELSVRAKP S (SEQ ID NO: 88).

In some embodiments, the decoy polypeptide comprises the amino acid sequence of SEQ ID NO: 72 or an amino acid sequence that is at least about 90%, 91%, 92%, 93%, 94%, 95%, 96%, 97%, 98% or 99% identical to SEQ ID NO: 72. In some embodiments, the decoy polypeptide comprises the amino acid sequence of SEQ ID NO: 90 or an amino acid sequence that is at least about 90%, 91%, 92%, 93%, 94%, 95%, 96%, 97%, 98% or 99% identical to SEQ ID NO: 90.

In a related aspect, provided is a decoy polypeptide comprising: (a) a SIRPβ2 variant and (b) a human Fc variant comprising at least one amino acid substitution that reduces effector function compared to a wild type human Fc, wherein the SIRPβ2 variant comprises at least one amino acid substitution relative to a wild type SIRPβ2, which substitution increases the affinity of the SIRPβ2 variant for CD47 as compared to the affinity of the wild type SIRPβ2 for CD47, and wherein the SIRPβ2 variant lacks a transmembrane domain. In some embodiments, the at least one amino acid substitution is within a d1 domain of the SIRPβ2 variant. In some embodiments, the amino acid sequence of the d1 domain of the SIRPβ2 variant is at least 90% identical to a sequence of a wild type SIRPβ2 d1 domain set forth in EEELQVIQPDKSISVAAGESATLHCTVTSLIPVGPIQWFRGAGPGRELIYNQKEGHFPR VTTVSDLTKRNNMDFSIRISNITPADAGTYYCVKFRKGSPDHVEFKSGAGTELSVRA KPS (SEQ ID NO: 27). In some embodiments, the SIRPβ2 variant comprises one or more amino acid substitutions at V6, V27, 131, E47, K53, E54, H56, L66, N80, V92 or H101, wherein the amino acid positions are relative to a wild-type human SIRPβ2 d1 domain sequence set forth in SEQ ID NO: 27. In some embodiments, the SIRPβ2 variant comprises the V6 substitution, and wherein the substitution is V6I. In some embodiments, the SIRPβ2 variant comprises the V27 substitution, and wherein the substitution is V27I. In some embodiments, the SIRPβ2 variant comprises the I31 substitution, and wherein the substitution is I31F. In some embodiments, the SIRPβ2 variant comprises the E47 substitution, and wherein the substitution is E47V. In some embodiments, the SIRPβ2 variant comprises the K53 substitution, and wherein the substitution is K53R. In some embodiments, the SIRPβ2 variant comprises the E54 substitution, and wherein the substitution is E54Q. In some embodiments, the SIRPβ2 variant comprises the H56 substitution, and wherein the substitution is H56P. In some embodiments, the SIRPβ2 variant comprises the L66 substitution, and wherein the substitution is L66T. In some embodiments, the SIRPβ2 variant comprises the N80 substitution, and wherein the substitution is N80A, N80C, N80D, N80E, N80F, N80G, N80H, N80I, N80K, N80L, N80M, N80P, N80Q, N80R, N80S, N80T, N80V, N80W, or N80Y. In some embodiments, the SIRPβ2 variant comprises the V92 substitution, and wherein the substitution is V92I. In some embodiments, the SIRPβ2 variant comprises the H101 substitution, and wherein the substitution is H101D. In some embodiments, the SIRPβ2 variant comprises the amino acid sequence of EEELQIIQPDKSISVAAGESATLHCTITSLFPVGPIQWFRGAGPGRVLIYNQRQGPF PRVTTVSDTTKRNNMDFSIRISNITPADAGTYYCIKFRKGSPDDVEFKSGAGTELS VRAKPS (SEQ ID NO: 28). In some embodiments, the SIRPβ2 variant comprises the amino acid sequence of EEELQIIQPDKSISVAAGESATLHCTITSLFPVGPIQWFRGAGPGRVLIYNQRQGPFPR VTTVSDTTKRNNMDFSIRISAITPADAGTYYCIKFRKGSPDDVEFKSGAGTELSVRAK PS (SEQ ID NO: 89).

In some embodiments, the decoy polypeptide comprises the amino acid sequence of SEQ ID NO: 73 or an amino acid sequence that is at least 90%, 91%, 92%, 93%, 94%, 95%, 96%, 97% 98% or 99% identical to SEQ ID NO: 73. In some embodiments, the decoy polypeptide comprises the amino acid sequence of SEQ ID NO: 91 or an amino acid sequence that is at least 90%, 91%, 92%, 93%, 94%, 95%, 96%, 97% 98% or 99% identical to SEQ ID NO: 91.

In some embodiments according to (or as applied to) any of the embodiments herein, the decoy polypeptide comprises a human Fc variant that comprises a modification that reduces glycosylation of the human Fc variant relative to a wild-type human Fc. In some embodiments, the glycosylation is reduced by enzymatic deglycosylation, expression in a bacterial host, or modification of an amino acid residue required for glycosylation. In some embodiments, the modification that reduces glycosylation of the human Fc variant comprises a substitution at N297, wherein numbering is according to the EU index of Kabat. In some embodiments, the substitution at N297 is N297A, N297Q, N297D, N297H, N297G, or N297C, wherein numbering is according to the EU index of Kabat. In some embodiments, the human Fc variant comprises substitutions at positions L234, L235, and/or G237, wherein numbering is according to the EU index of Kabat. In some embodiments, the human Fc variant comprises L234A and L235A substitutions, wherein numbering is according to the EU index of Kabat. In some embodiments, the Fc variant further comprises a K322A substitution, wherein numbering is according to the EU index of Kabat. In some embodiments, the modification to the human Fc comprises E233P, L234V, L235A, delG236, A327G, A330S, and P331S mutations, wherein numbering is according to the EU index of Kabat. In some embodiments, the human Fc variant is selected from the group consisting of: (a) a human IgG1 Fc comprising L234A, L235A, G237A, and N297A substitutions, wherein numbering is according to the EU index of Kabat; (b) a human IgG2 Fc comprising A330S, P331S, and N297A substitutions, wherein numbering is according to the EU index of Kabat; and (c) a human IgG4 Fc comprising S228P, E233P, F234V, L235A, delG236, and N297A mutations wherein numbering is according to the EU index of Kabat. In some embodiments, the human Fc variant is a human IgG1 Fc comprising L234A, L235A, G237A, and N297A substitutions wherein numbering is according to the EU index of Kabat. In some embodiments, the human Fc is a human IgG1 Fc comprising (such as further comprising) a D265A substitution, wherein numbering is according to the EU index of Kabat. In some embodiments, the human Fc variant exhibits ablated or reduced binding to an Fcγ receptor as compared to a wild-type human IgG1 Fc. In some embodiments, the human Fc variant exhibits ablated or reduced binding to CD16a, CD32a, CD32b, CD32c, and CD64 Fcγ receptors as compared to a wild-type human IgG1 Fc. In some embodiments, the human Fc variant exhibits ablated or reduced binding to C1q compared to a wild-type human IgG1 Fc. In some embodiments, the human Fc variant is a human IgG2 Fc comprising A330S, P331S, and N297A substitutions, wherein numbering is according to the EU index of Kabat. In some embodiments, the human Fc variant exhibits ablated or reduced binding to an Fcγ receptor as compared to a wild-type human IgG2 Fc. In some embodiments, the human Fc variant exhibits ablated or reduced binding to CD16a, CD32a, CD32b, CD32c, and CD64 Fcγ receptors as compared to a wild-type human IgG2 Fc. In some embodiments, the human Fc variant exhibits ablated or reduced binding to C1q compared to a wild-type human IgG2 Fc. In some embodiments, the human Fc variant is a human IgG4 Fc comprising S228P, E233P, F234V, L235A, delG236, and N297A mutations, wherein numbering is according to the EU index of Kabat. In some embodiments, the human Fc variant is a human IgG4 Fc comprising an S228P substitution, wherein numbering is according to the EU index of Kabat. In some embodiments, the human Fc variant is a human IgG4 Fc comprising S228P and L235E substitutions, wherein numbering is according to the EU index of Kabat. In some embodiments, the human Fc variant exhibits ablated or reduced binding to an Fcγ receptor as compared to a wild-type human IgG4 Fc. In some embodiments, the human Fc variant exhibits ablated or reduced binding to CD16a and CD32b Fcγ receptors compared to the wild-type version of its human IgG4 Fc. In some embodiments, the human Fc variant comprises an amino acid sequence set forth in any one of SEQ ID NOs: 48-51, 53-56, 93-96, and 98-101. In some embodiments, the human Fc variant binds to an Fcγ receptor with a K_{D} greater than about 5×10⁻⁶ M. In some embodiments, the decoy polypeptide does not cause acute anemia in rodents and non-human primates following administration. In some embodiments, the decoy polypeptide does not cause acute anemia in humans following administration.

In some embodiments, the decoy polypeptide blocks binding of CD47 to a ligand. In some embodiments, the CD47 is a human CD47, a CD47 of a non-human primate (e.g., cynomolgus monkey), or a mouse CD47. In some embodiments, the ligand is SIRPα or SIRPγ. In some embodiments, the decoy polypeptide binds to CD47 expressed on the surface of a cell. In some embodiments, the cell is a tumor cell, virally infected cell, bacterially infected cell, damaged red blood cell, arterial plaque cell, fibrotic tissue cell, a healthy normal cell such as hematopoietic stem cell. In some embodiments, the binding of the decoy polypeptide to CD47 expressed on the surface of the cell induces or enhances phagocytosis or ADCC of the cell, e.g., tumor cell, virally infected cell, bacterially infected cell, damaged red blood cell, arterial plaque cell, or fibrotic tissue cell. In some embodiments, the decoy polypeptide is a dimer. In some embodiments, the dimer is a homodimer. In some embodiments, the decoy polypeptide further comprises a detectable label.

In a related aspect, provided is a composition comprising the decoy polypeptide according to (or as applied to) any of the embodiments disclosed herein and a pharmaceutically acceptable excipient. In some embodiments, the composition further comprises one or more additional agents. In some embodiments, the one or more additional agents is a chemotherapeutic agent, a kinase inhibitor, a proteasome inhibitor, an inhibitor of a viral DNA polymerase, an inhibitor of a viral RNA polymerase, or a therapeutic antibody. In some embodiments, the one or more additional agents is a therapeutic antibody. In some embodiments, the therapeutic antibody is cetuximab, necitumumab, pembrolizumab, nivolumab, pidilizumab, ipilimumab, tremelimumab, urelumab, daratumumab, trastuzumab, trastuzumab emtansine, pertuzumab, elotuzumab, rituximab, ofatumumab, obinutuzumab, panitumumab, brentuximab vedotin, MSB0010718C, belimumab, bevacizumab, denosumab, ramucirumab, atezolizumab. In some embodiments, the therapeutic antibody targets a HLA/peptide or MHC/peptide complex comprising a peptide derived from NY-ESO-1/LAGE1, SSX-2, a member of the MAGE protein family, gp100/pmel17, MelanA/MART1, gp75/TRP1, tyrosinase, TRP2, CEA, PSA, TAG-72, Immature laminin receptor, MOK/RAGE-1, WT-1, Her2/neu, EphA3, SAP-1, BING-4, Ep-CAM, MUC1, PRAME, survivin, Mesothelin, BRCA1, BRCA2, CDK4, CML66, MART-2, p53, Ras, (3-catenin, TGF-βRII, HPV E6, or HPV E7. In some embodiments, the therapeutic antibody binds an antigen on a cancer cell, an immune cell, a pathogen-infected cell, or a hematopoietic stem cell. In some embodiments, the therapeutic antibody binds an antigen on a cancer cell, and wherein the antigen is EGFR, Her2/neu, CD19, CD20, CD22, CD25, CD30, CD33, CD38, CD45, CD47, CD56, CD70, CD117, or EpCAM. In some embodiments, the therapeutic antibody binds an antigen on an immune cell, and wherein the antigen is M1prime, CD2, CD3, CD4, CD5, CD8, CD19, CD20, CD22, CD25, CD38, CD56, PD-1, PD-L1, CTLA4, BTLA, TIM3, LAG3, OX40, GITR or CD137 (4-1BB). In some embodiments, the therapeutic antibody binds an antigen on a pathogen-infected cell, and wherein the antigen is a CMV protein, UL18, UL11, pp65, gB, pp150, an HIV envelope protein, Gp41, Gp120, V1V2 glycan, V3 glycan, and influenza hemagglutinin. In some embodiments, the therapeutic antibody binds an antigen on a hematopoietic stem cell, and wherein the antigen is CD11, CD45, CD117 or Sca1.

Also provided is an isolated nucleic acid encoding the decoy polypeptide according to (or as applied to) any of the embodiments herein. Further provided is a vector comprising such a nucleic acid. Provided is a host cell comprising a nucleic acid or a vector according to (or as applied to) any of the embodiments herein. The present disclosure provides a method of producing a decoy polypeptide, comprising culturing a host cell of claim according to (or as applied to) any of the embodiments herein under conditions where the decoy polypeptide is expressed and recovering the decoy polypeptide.

In a related aspect, provided is a method of modulating phagocytosis or ADCC of a cell expressing CD47, the method comprising contacting the cell with a decoy polypeptide according to (or as applied to) any of the embodiments herein or a composition according to (or as applied to) any of the embodiments herein. Also provided is a method of treating a subject having a disease or disorder, comprising administering an effective amount of a decoy polypeptide according to (or as applied to) any of the embodiments herein or a composition according to (or as applied to) any of the embodiments herein to the subject. In some embodiments, the disease or disorder is cancer, anemia, a viral infection, a bacterial infection, an autoimmune disease or an inflammatory disorder, asthma, an allergy, a transplant rejection, atherosclerosis, or fibrosis. In some embodiments, the disease or disorder is cancer, and wherein the cancer is cancer is solid tumor, hematological cancer, acute myeloid leukemia, chronic lymphocytic leukemia, chronic myeloid leukemia, acute lymphoblastic leukemia, non-Hodgkin lymphoma, Hodgkin lymphoma, multiple myeloma, bladder cancer, pancreatic cancer, cervical cancer, endometrial cancer, lung cancer, bronchus cancer, liver cancer, ovarian cancer, colon and rectal cancer, stomach cancer, gastric cancer, gallbladder cancer, gastrointestinal stromal tumor cancer, thyroid cancer, head and neck cancer, oropharyngeal cancer, esophageal cancer, melanoma, non-melanoma skin cancer, Merkel cell carcinoma, virally induced cancer, neuroblastoma, breast cancer, prostate cancer, renal cancer, renal cell cancer, renal pelvis cancer, leukemia, lymphoma, sarcoma, glioma, brain tumor, and carcinoma. In some embodiments, the disease or disorder is an autoimmune disease or inflammatory disorder, and wherein the autoimmune disease or inflammatory disorder is multiple sclerosis, rheumatoid arthritis, a spondyloarthropathy, systemic lupus erythematosus, an antibody-mediated inflammatory or autoimmune disease, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, and inflammatory autoimmune myositis.

Provided is a decoy polypeptide according to (or as applied to) any of the embodiments herein or a composition according to (or as applied to) any of the embodiments herein for use in treating cancer, viral infection, bacterial infection, auto-immune disease, asthma, allergy, transplant rejection, atherosclerosis, or fibrosis. Provided is a decoy polypeptide according to (or as applied to) any of the embodiments herein or a composition according to (or as applied to) any of the embodiments herein for use in preconditioning for a hematopoietic stem cell transplant.

In a related aspect, the present disclosure provides a method of detecting a CD47⁺ cell in a population of cells, comprising contacting the population of cells with a decoy polypeptide according to (or as applied to) any of the embodiments herein or a composition according to (or as applied to) any of the embodiments herein and detecting binding of the decoy polypeptide to CD47⁺ cells, wherein the detecting of the binding indicates the presence of CD47⁺ cells. In some embodiments, the cells are tumor cells, virally infected cells, bacterially infected cells, autoreactive T or B cells, damaged red blood cells, arterial plaque cells, or fibrotic tissue cells. In some embodiments, the contacting is in vivo. In some embodiments, the contacting is in vitro. The present disclosure also provides a method of purifying a CD47⁺ cell from a population of cells, comprising contacting the population of cells with a decoy polypeptide according to (or as applied to) any of the embodiments herein and isolating the cells bound to the decoy polypeptide.

Also provided is a chimeric molecule comprising a decoy polypeptide according to (or as applied to) any of the embodiments herein and an immune checkpoint inhibitor, a co-stimulatory molecule, a cytokine, or an attenuated cytokine. In some embodiments, the decoy polypeptide is linked to the immune checkpoint inhibitor, co-stimulatory molecule, cytokine, or attenuated cytokine through a linker sequence. In some embodiments, the linker sequence comprises Gly and Ser. In some embodiments, the linker sequence comprises GGGGSGGGGS (SEQ ID NO: 29). In some embodiments, the decoy polypeptide is fused to the N-terminal or C-terminal end of the immune checkpoint inhibitor, co-stimulatory molecule, cytokine, or attenuated cytokine. In some embodiments, the decoy polypeptide is fused to an immune checkpoint inhibitor, and wherein the immune checkpoint inhibitor comprises a sequence of a PD-1 or PD-L1 antagonist, a BTLA or CD160 antagonist, a phosphatidylserine antagonist, MFGE8, TIM1, TIM3, or TIM4. In some embodiments, the decoy polypeptide is fused to a co-stimulatory molecule, and wherein the co-stimulatory molecule comprises a sequence of a CD40 agonist, a 41BBL or CD137 agonist. In some embodiments, the decoy polypeptide is fused to a cytokine, and wherein the cytokine comprises a sequence of an IL2. In some embodiments, the IL2 sequence comprises mutations D20T and F42A. In some embodiments, the decoy polypeptide is fused to a cytokine polypeptide, and wherein the cytokine is attenuated. In some embodiments, the chimeric molecule comprises an amino acid sequence set forth in SEQ ID NO: 30 or SEQ ID NO: 102. In some embodiments, the chimeric molecule comprises an amino acid sequence set forth in SEQ ID NO: 31 or SEQ ID NO: 103. In some embodiments, the chimeric molecule comprises an amino acid sequence set forth in any one of SEQ ID NOs: 32-39 or SEQ ID NO: 104-111.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1B show SDS-PAGE analyses to determine the expression of decoy polypeptides under non-reducing and reducing conditions. FIG. 1A shows SDS-PAGE analysis of decoy polypeptides A, C, J, and P under non-reducing and reducing conditions.

FIG. 1B shows SDS-PAGE analysis of decoy polypeptides Q, R, S, and T under non-reducing and reducing conditions. In FIGS. 1A-1B, a protein molecular weight marker was used to determine the molecular weights of the observed bands (kDa) and a negative control of Expi293 cells that were mock-transfected (“no DNA”) was used.

FIG. 2 provides a summary of sequence alignment analyses of SIRPγ, SIRPβ1, SIRPβ2, and SIRPα D1 domain variants used to generate the decoy polypeptides described in Example 1. The percent amino acid similarity is shown on the horizontal axis and the percent amino acid identity is shown on the vertical axis.

FIGS. 3A-3B show amino acid sequence differences between a wild type SIRPβ1 D1 domain and SIRPβ1 D1 domain variant of decoy polypeptide P described in Example 1. FIG. 3A provides a sequence alignment of a SIRPβ1 D1 domain variant comprising the sequence of SEQ ID NO: 26 and a wild type SIRPβ1 D1 domain (SEQ ID NO: 25). Residues indicated by arrows show positions that differed between the variant and wild type amino acid sequences. FIG. 3B shows the SIRPβ1 D1 domain X-ray crystal structure (PDB: 2JJU) superimposed onto a crystal structure of the SIRPα D1 domain bound to CD47 (PDB: 2JJS). Amino acids that differed between wild type and variant SIRPβ1 D1 domains sequences are shown as spheres.

FIGS. 4A-4B show amino acid sequence differences between a wild type SIRPβ2 D1 domain and the SIRPβ2 D1 domain variant of decoy polypeptide Q described in Example 1. FIG. 4A provides a sequence alignment of a SIRPβ2 D1 domain variant comprising the sequence of SEQ ID NO: 28 and a wild type SIRPβ2 D1 domain (SEQ ID NO: 27). Residues indicated by arrows show positions that differed between the variant and wild type amino acid sequences. FIG. 4B shows the SIRPβ2 D1 domain X-ray crystal structure (PDB: 2JJV) superimposed onto a crystal structure of the SIRPα D1 domain bound to CD47 (PDB: 2JJS). Amino acids that differed between wild type and variant SIRPβ2 D1 domains sequences are shown as spheres.

FIGS. 5A-5D show amino acid sequence differences between the SIRPγ D1 domain variants of decoy polypeptides A-O described in Example 1. FIG. 5A provides a sequence alignment of SIRPγ D1 domain variants comprising SEQ ID NOs: 3-8, 10-11, 13, 17-19, 21-22, and 42. Residues denoted with stars differed among the SIRPγD1 domain variants. FIG. 5B provides a sequence alignment of a wild type SIRPγ D1 domain (SEQ ID NO: 1) and four SIRPγ D1 domain variants (SEQ ID NOs: 4, 5, 11, and 17) that demonstrated the highest affinities for hCD47 among the SIRPγ D1 domain variants that were tested. Arrows indicate residues that were substituted in the variants relative to wild type SIRPγ D1 domains. Additionally, FIG. 5B provides a sequence alignment of a wild type SIRPα D1 domain (SEQ ID NO: 81) and an exemplary SIRPα D1 domain variant (SEQ ID NO: 78). FIG. 5C shows a crystal structure of the SIRPγ D1 domain bound to CD47 (PDB: 2JJW). FIG. 5D shows the five amino acid residues that were mutated in the variant SIRPγ D1 domains (FIG. 5B) as spheres on a crystal structure of the SIRPγ D1 domain bound to CD47.

FIG. 6 provides an alignment of the sequences of wild type SIRPα (SEQ ID NO: 81), SIRPβ1 (SEQ ID NO: 25), SIRPβ2 (SEQ ID NO: 27), and SIRPγ (SEQ ID NO: 1) D1 domains. Residues that were substituted in the SIRPα, SIRP1, SIRPβ2, and SIRPγ D1 domain variants that demonstrated improved binding to hCD47 are bolded. Boxed regions indicate the regions of human SIRPα that bind to human CD47. Arrows indicate amino acid positions that were substituted in each of the SIRPα, SIRP1, SIRPβ2, and SIRPγ D1 domain variants that exhibited improved binding to CD47 relative to wild type.

FIGS. 7A-7B show the results of in vitro experiments that were performed to determine the effect of decoy polypeptides in combination with cetuximab (CTX; 10 ng/ml) on the phagocytosis of CFSE-labeled DLD-1 tumor cells by human monocyte-derived macrophages. FIG. 7A shows the effect of decoy polypeptides P, Q, S, T, and U on the phagocytosis of tumor cells by macrophages. FIG. 7B shows the effect of decoy polypeptides A, C, J, R, and U on the phagocytosis of tumor cells by macrophages. In FIGS. 7A-7B, the level of phagocytosis is indicated on the y-axis, as the percent of macrophages that phagocytosed tumor cells and were CFSE+; the concentration of decoy polypeptide is indicated on the x-axis (nM); cells were also incubated with 10 ng/mL cetuximab alone, control hIgG antibody, and no antibody (“Media only”).

FIGS. 8A-8D show the results of experiments that were performed to determine the effect of administration of decoy polypeptide V or decoy polypeptide C in hematological parameters in mice. The time points at which each hematological parameter was measured were 8 hours prior to administration of the decoy polypeptide (i.e., “−8”), 3 days following administration, and 8 days following administration. FIG. 8A shows the effect of administration of decoy polypeptides V and C on white blood cell (WBC: lymphocytes, monocytes, and granulocytes) levels in mice. FIG. 8B shows the effect of administration of decoy polypeptides V and C on lymphocyte levels in mice. FIG. 8C shows the effect of administration of decoy polypeptides V and C on monocyte levels in mice. FIG. 8C shows the effect of administration of decoy polypeptides V and C on platelet (PLT) levels in mice.

### DETAILED DESCRIPTION OF THE INVENTION

### Definitions

The term “decoy polypeptide,” as used herein refer to fusion polypeptides comprising (a) a SIRPγ variant, a SIRPβ1 variant, or a SIRPβ2 variant and (b) a human Fc variant comprising at least one amino acid substitution that reduces effector function compared to a wild type human Fc. The decoy polypeptide prevents binding of CD47 to its ligand (e.g., SIRPα or SIRPγ) in vitro and/or in vivo. For development purposes the binding may be performed under experimental conditions, e.g. using isolated proteins as ligands, using portions of proteins as ligands, using yeast display of proteins or portions of proteins as ligands, and the like. For physiologically relevant purposes the binding of CD47 to its ligands is often an event between two cells, where each cell expresses one of the binding partners. Of particular interest is the expression of SIRP polypeptides on phagocytotic cells, such as macrophages; and the expression of CD47 on cells that could be targets for phagocytosis, e.g. tumor cells, circulating hematopoietic cells, and the like. Decoy polypeptides may be identified using in vitro and in vivo assays for receptor or ligand binding or signaling.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” as used herein refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. The term “amino acid analogs” as used herein refers to compounds that have the same basic chemical structure as a naturally occurring amino acid, i.e., an alpha carbon that is bound to a hydrogen, a carboxyl group, an amino group, and an R-group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” as used herein refers to chemical compounds that have a structure that is different from the general chemical structure of an amino acid, but which functions in a manner similar to a naturally occurring amino acid.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sport, or pet animals, such as dogs, horses, cats, cows, sheep, goats, pigs, mice, rats, rabbits, guinea pigs, monkeys etc. In some embodiments, the mammal is human.

As used herein “cancer” includes any form of cancer, including, but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

The term “binding partner” as used herein refers to a member of a specific binding pair (i.e., two molecules, usually two different molecules, where one of the molecules, e.g., a first binding partner, through non-covalent means specifically binds to the other molecule, e.g., a second binding partner).

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The term “antibody” is used in the broadest sense and specifically covers monoclonal antibodies (including full length monoclonal antibodies), polyclonal antibodies, multispecific antibodies (e.g., bispecific antibodies), peptibodies, human antibodies, humanized antibodies, camelid antibodies (including camelid single domain antibodies), alternative scaffold antibodies (e.g., affibodies, avimers, Fn3 domains, DARPins, Kunitz domains, SMIPs, Domain antibodies, BiTEs, Adnectins, Nanobodies, Stable scFvs, Anticalins) and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

### Decoy Polypeptides

Provided are compositions and methods relating to decoy polypeptides that comprise (a) a SIRPγ variant, a SIRPβ1 variant, or a SIRPβ2 variant; and (b) a human Fc variant comprising at least one amino acid substitution that reduces effector function compared to a wild type human Fc or ablates effector function. The decoy polypeptides provided herein block the binding of CD47 (e.g., human CD47, a CD47 from a non-human primate, such as a cynomolgus monkey, or mouse CD47) to a ligand e.g., SIRPα (from a human, non-human primate, or mouse) or SIRPγ (from a human, non-human primate, or mouse). Blocking the binding of CD47 and SIRPα pathway mediates phagocytosis of targeted cells and can synergize with other cell targeting agents, including, e.g., cancer-specific antibodies, pathogen specific antibodies, and the like. Fc-containing polypeptides that target cell surface antigens can trigger immunostimulatory and effector functions that are associated with Fc receptor (FcR) engagement on immune cells. There are a number of Fc receptors that are specific for particular classes of antibodies, including IgG (gamma receptors), IgE (eta receptors), IgA (alpha receptors) and IgM (mu receptors). Binding of the Fc region to Fc receptors on cell surfaces can trigger a number of biological responses including phagocytosis of antibody-coated particles (antibody-dependent cell-mediated phagocytosis, or ADCP), clearance of immune complexes, lysis of antibody-coated cells by killer cells (antibody-dependent cell-mediated cytotoxicity, or ADCC) and, release of inflammatory mediators, placental transfer, and control of immunoglobulin production. Additionally, binding of the C1q component of complement to the Fc can activate the complement system. Activation of complement can be important for the lysis of cellular pathogens. However, the activation of complement can also stimulate the inflammatory response and can also be involved in autoimmune hypersensitivity or other immunological disorders. Human Fc variants with reduced or ablated ability to bind certain Fc receptors and/or C1q are useful for developing and Fc-fusion polypeptide constructs which act by blocking, targeting, activating, or neutralizing ligand functions while not damaging or destroying local cells or tissues. Generally, the human Fc variants are designed to have mutations that perturb binding to Fc gamma receptors and C1q but the human Fc variants retain binding to FcRn.

In some embodiments, the decoy polypeptide comprises (a) a soluble SIRPγ variant (i.e., a SIRPγ variant lacking a transmembrane domain), a soluble SIRPγ variant (i.e., a SIRPγ variant lacking a transmembrane domain), or a soluble SIRPβ2 variant (i.e., a SIRPβ2 variant lacking a transmembrane domain), and (b) a human Fc variant that comprises a modification (e.g., one or more amino acid substitutions) that reduces binding to a human Fc receptor and C1q protein or ablates binding to a human Fc receptor and C1q protein. In some embodiments, the human Fc variant exhibits ablated or reduced binding to Fc receptors, including human Fcγ receptors, relative to a wild-type Fc region.

In some embodiments, the C-terminus of the SIRPγ variant (such as a soluble SIRPγ variant), SIRPγ variant (such as a soluble SIRPγ variant), or SIRPβ2 variant (such as a soluble SIRPβ2 variant) is joined to the N-terminus of the human Fc variant. In some embodiments, the C-terminus of the SIRPγ variant (such as a soluble SIRPγ variant), SIRPβ1 variant (such as a soluble SIRPβ1 variant), or SIRPβ2 variant (such as a soluble SIRPβ2 variant) is joined to the N-terminus of the human Fc variant by way of a linker using conventional genetic or chemical means, e.g., chemical conjugation. In some embodiments, a linker (e.g., a spacer) is inserted between the SIRPγ variant (such as a soluble SIRPγ variant), SIRPβ1 variant (such as a soluble SIRPβ1 variant), or SIRPβ2 variant (such as a soluble SIRPβ2 variant) and the human Fc variant.

In some embodiments, the SIRPγ variant (such as a soluble SIRPγ variant), SIRPβ1 variant (such as a soluble SIRPβ1 variant), or SIRPβ2 variant (such as a soluble SIRPβ2 variant) variant is fused to a human Fc variant that is incapable of forming a dimer. In some embodiments, the SIRPγ variant (such as a soluble SIRPγ variant), SIRPβ1 variant (such as a soluble SIRPβ1 variant), or SIRPβ2 variant (such as a soluble SIRPβ2 variant) is fused to a human Fc variant that is capable of forming a dimer, e.g., a heterodimer or a homodimer, with a second human Fc variant.

In some embodiments, the decoy polypeptide is a dimer. In some embodiments, the dimer is a homodimer. In some embodiments, the dimer is a heterodimer. In some embodiments, the heterodimer comprises, e.g., a first decoy polypeptide comprising a first human Fc variant and a second decoy polypeptide comprising a second human Fc variant. Additionally or alternatively, in some embodiments, the heterodimer comprises, e.g., a first decoy polypeptide that comprises a first SIRPγ variant and a second decoy polypeptide that comprises a second SIRPγ variant, a first decoy polypeptide that comprises a first SIRPβ1 variant and a second decoy polypeptide that comprises a second SIRPβ1 variant, or a first decoy polypeptide that comprises a first SIRPβ2 variant and a second decoy polypeptide that comprises a second SIRPβ2 variant. In some embodiments, the heterodimer comprises, e.g., a first decoy polypeptide that comprises a SIRPγ variant and a second decoy polypeptide that comprises a SIRPα variant, a SIRPβ1 variant, or a SIRP SIRPβ2 variant. In some embodiments, the heterodimer comprises, e.g., a first decoy polypeptide that comprises a SIRPβ1 variant and a second decoy polypeptide that comprises a SIRPα variant or a SIRPβ2 variant. In some embodiments, the heterodimer comprises, e.g., a first decoy polypeptide that comprises a SIRPβ2 variant and a second decoy polypeptide that comprises a SIRPα variant. Except where indicated otherwise by context, the terms “first decoy polypeptide” and “second decoy polypeptide” are merely arbitrary designations and that “first” and “second” in any of the embodiments described herein can be reversed. Exemplary SIRPα variants are disclosed in, e.g., WO 2013/109752, WO 2016/023040, WO 2017/027422, and WO 2014/094122, the disclosures of all of which are incorporated herein by reference in their entirety.

In some embodiments, the decoy polypeptide binds CD47. In some embodiments, the decoy polypeptide binds to CD47 expressed on the surface of a cell. In some embodiments, decoy polypeptide binds to CD47 expressed on the surface of, e.g., a tumor cell, a virally infected cell, a bacterially infected cell, a self-reactive cell (e.g., a self-reactive T cell or self-reactive B cell) or other undesirable or pathogenic cell in the body (e.g., a damaged red blood cell, an arterial plaque, or fibrotic tissue cells). In some embodiments, binding of the decoy polypeptide to CD47 blocks binding of CD47 to a binding partner or ligand. In some embodiments, the CD47 binding partner or ligand is SIRPα (SIRPA) and/or SIRPγ (SIRPG). In some embodiments, binding of the decoy polypeptide to CD47 (e.g., CD47 expressed on the surface of a cell) activates, enhances, induces, or causes phagocytosis of the cell by a phagocyte, such as a professional phagocyte (e.g., a monocyte, a macrophage, a neutrophil, a dendritic cell, and/or a mast cell) and/or a non-professional phagocyte (e.g., an epithelial cell, an endothelial cell, a fibroblast, and/or a mesenchymal cell).

In some embodiments, the decoy polypeptide comprises a soluble SIRPγ variant, a soluble SIRPβ1 variant, or a soluble SIRPβ2 variant in multimeric form. In some embodiments, the decoy polypeptide comprises a dimer (e.g., a homodimer or a heterodimer), a trimer, a tetramer, a pentamer or other multimer. In some embodiments, the decoy polypeptide comprises a soluble SIRPγ variant, a soluble SIRPβ1 variant, or a soluble SIRPβ2 variant in monomeric form. In some embodiments, the decoy polypeptide is multispecific (e.g., capable of binding CD47 and a second target). In some embodiments, the decoy polypeptide comprises a multi-specific SIRPγ variant, a multispecific SIRPβ1 variant, or a multispecific SIRPβ2 variant.

In some embodiments, the off rate of a decoy polypeptide comprising a soluble SIRPγ variant is decreased by at least about any one of 10-fold, 20-fold, 50-fold 100-fold 500-fold, 750-fold, 1,000-fold, 2,000-fold, 3,000-fold, 4,000-fold, 5,000-fold, 6,000-fold, 7,000-fold, 8,000-fold, 9,000-fold, 10,000-fold or more, as compared to a polypeptide comprising a wild type SIRPγ lacking a transmembrane domain, including any range in between these values. In some embodiments, the off rate of a decoy polypeptide comprising a soluble SIRPβ1 variant is decreased by at least 10-fold, at least 20-fold, at least 50-fold, at least 100-fold, at least 500-fold, or more, as compared to a polypeptide comprising a wild type SIRPβ1 lacking a transmembrane domain, including any range in between these values. In some embodiments, the off rate of a decoy polypeptide comprising a soluble SIRPβ2 variant is decreased by at least 10-fold, at least 20-fold, at least 50-fold, at least 100-fold, at least 500-fold, or more, as compared to a polypeptide comprising a wild type SIRPβ2 lacking a transmembrane domain, including any range in between these values.

In some embodiments, the decoy polypeptides described herein stimulate and/or enhance phagocytosis and/or ADCC by myeloid cells (e.g., macrophages, monocytes, dendritic cells, neutrophils, etc.) to eliminate pathogenic cells (e.g., tumor cells, virally or bacterially infected cells, autoreactive T cells, etc.). In some embodiments, cells are eliminated selectively, thereby reducing the potential for toxic side effects. In some embodiments, the decoy polypeptides are used to enhance the elimination of endogenous cells for therapeutic effect, such as B or T lymphocytes in autoimmune disease, asthma, and allergy, or hematopoietic stem cells (HSCs) for stem cell transplantation.

In some embodiments, the decoy polypeptides described herein exhibit increased occupancy or receptor occupancy compared to other antagonists of the interaction between CD47: SIRPα that are known in the art. In some embodiments, the decoy polypeptides described herein exhibit increased persistence compared to other known antagonists of the interaction between CD47: SIRPα. Occupancy, or receptor occupancy, as used herein, refers to binding to a target cell, target receptor, target protein, or target tissue. Persistence, as used herein, refers to serum half-life or cell binding half-life of the decoy polypeptides when administered to an individual, subject, or patient.

In some embodiments, the decoy polypeptide has an increased affinity for CD47 (e.g., human CD47) as compared to the affinity of a wild type SIRPγ, a wild type SIRPβ1 or a wild type SIRPβ2 for CD47 (e.g., human CD47).

In some embodiments, the decoy polypeptide comprises a SIRPγ variant (e.g., a soluble SIRPγ variant), a SIRPβ1 variant (e.g., a soluble SIRPβ1 variant), or a SIRPβ2 variant (e.g., a soluble SIRPβ2 variant) that has a K_{d} of about 1×10⁻⁷ M or less (e.g., any one of about 1×10⁻⁸ M or less, 1×10⁻⁹ M or less, 1×10⁻¹⁰ M or less, 1×10⁻¹¹ M or less, 1×10⁻¹² M or less, 1×10⁻¹³ M or less, 1×10⁻¹⁴ M or less, 1×10⁻¹⁵ M or less, or 1×10⁻¹⁶ M or less) affinity for CD47. In some embodiments, the decoy polypeptide comprises a SIRPγ variant (e.g., a soluble SIRPγ variant), a SIRPβ1 variant (e.g., a soluble SIRPβ1 variant), or a SIRPβ2 variant (e.g., a soluble SIRPβ2 variant) that has an affinity for CD47 in a range of from 1 fM to 1 μM (e.g., from 1 fM to 800 nM, from 10 fM to 500 nM, from 100 fM to 100 nM, from 500 fM to 50 nM, from 800 fM to 50 nM, from 1 pM to 50 nM, from 10 pM to 50 nM, from 50 pM to 50 nM, from 100 pM to 50 nM, from 500 fM to 100 nM, from 800 fM to 100 nM, from 1 pM to 100 nM, from 10 pM to 100 nM, from 50 pM to 100 nM, or from 100 pM to 100 nM). In some embodiments, the decoy polypeptide comprises a SIRPβγ variant (e.g., a soluble SIRPγ variant), a SIRPβ1 variant (e.g., a soluble SIRPβ1 variant), or a SIRPβ2 variant (e.g., a soluble SIRPβ2 variant) that binds to CD47 with an affinity of 1 μM or greater (e.g., 800 nM or greater, 500 nM or greater, 200 nM or greater, 100 nM or greater, 50 nM or greater, 10 nM or greater, 1 nM or greater, 900 pM or greater, 750 pM or greater, 500 pM or greater, 200 pM or greater, 100 pM or greater, 10 pM or greater, 1 pM or greater, etc., where the affinity increases with decreasing values).

In some embodiments, the decoy polypeptide comprises a SIRPγ variant (e.g., a soluble SIRPγ variant), a SIRPβ1 variant (e.g., a soluble SIRPβ1 variant), or a SIRPβ2 variant (e.g., a soluble SIRPβ2 variant) has an affinity for CD47 that is at least about 2-fold greater or more (e.g., at least about any one of 5-fold greater, 10-fold greater, 100-fold greater, 500-fold greater, 1000-fold greater, 5000-fold greater, 10⁴-fold greater, 10⁵-fold greater, 10⁶-fold greater, 10⁷-fold greater, 10⁸-fold greater or more, etc., including any range in between these values) than the affinity for CD47 of a wild type SIRPγ, a wild type SIRPβ1 or a wild type SIRPβ2 protein.

In some embodiments, the decoy comprises a SIRPγ variant (e.g., a soluble SIRPγ variant), a SIRPβ1 variant (e.g., a soluble SIRPβ1 variant), or a SIRPβ2 variant (e.g., a soluble SIRPβ2 variant) that has a dissociation half-life for CD47 that is 2-fold greater or (e.g., about any one of 5-fold greater, 10-fold greater, 100-fold greater, 500-fold greater, 1000-fold greater, 5000-fold greater, 10⁴-fold greater, 10⁵-fold greater, 10⁶-fold greater, 10⁷-fold greater, 10⁸-fold greater or more, etc., including any range in between these values) greater than the dissociation half-life for CD47 of a wild type SIRPγ, a wild type SIRPβ1 or a wild type SIRPβ2. For example, in some cases, a wild type SIRPγ, a wild type SIRP1, or a wild type SIRPβ2 polypeptide has a dissociation half-life for CD47 of less than 1 second, while a decoy polypeptide described herein comprises a SIRPγ variant (e.g., a soluble SIRPγ variant), a SIRPβ1 variant (e.g., a soluble SIRPβ1 variant), or a SIRPβ2 variant (e.g., a soluble SIRPβ2 variant) that has a dissociation half-life of 5 seconds or more (e.g., 30 seconds or more, 1 minute or more, 5 minutes or more, 10 minutes or more, 20 minutes or more, 30 minutes or more, 40 minutes or more, etc., including any range in between these values). For example, in some embodiments, the amino acid substitution(s)/deletions/insertions in a comprises a SIRPγ variant (e.g., a soluble SIRPγ variant), a SIRPβ1 variant (e.g., a soluble SIRPβ1 variant), or a SIRPβ2 variant (e.g., a soluble SIRPβ2 variant) increase the affinity of the decoy polypeptide for binding to CD47 (e.g., as compared to a wild type SIRPγ, a wild type SIRPβ1 or a wild type SIRPβ2, respectively) by decreasing the off-rate by at least 10-fold, at least 20-fold, at least 50-fold, at least 100-fold, at least 500-fold, or more, including any range in between.

The affinity to bind to CD47 can be determined, for example, by the ability of the decoy polypeptide to bind to CD47 coated on an assay plate; displayed on a microbial cell surface; in solution; etc. The binding activity of decoy polypeptides provided herein to CD47 can be assayed by immobilizing the ligand (e.g., CD47) or the decoy polypeptide to a bead, substrate, cell, etc. Agents can be added in an appropriate buffer and the binding partners incubated for a period of time at a given temperature. After washes to remove unbound material, the bound binding partner can be released with, for example, SDS, buffers with a high pH, and the like and analyzed, for example, by Surface Plasmon Resonance (SPR).

Binding can also be determined by, for example, measuring the ability of a unlabeled decoy polypeptide to compete with a labeled polypeptide comprising the extracellular domain (or a portion thereof) of a wild type SIRPγ, a wild type SIRP1, or a wild type SIRPβ2 polypeptide and a human Fc variant for binding to CD47. Accordingly, relative biding can be assessed by comparing the results using a candidate unlabeled decoy polypeptide to results using an unlabeled polypeptide comprising a wild type SIRPγ, a wild type SIRP1, or a wild type SIRPβ2 and a human Fc variant.

### SIRPγ Variants, SIRPβ1 Variants, and SIRPβ2 Variants

In some embodiments, the decoy polypeptides provided herein comprise (a) a soluble SIRPγ variant (i.e., a variant lacking a transmembrane domain), a soluble SIRPβ1 variant (i.e., a variant lacking a transmembrane domain), or a soluble SIRPβ2 variant (i.e., a variant lacking a transmembrane domain), and (b) a human Fc variant.

Signal regulatory proteins (SIRPs) constitute a family of cell surface glycoproteins which are expressed on myeloid (including macrophages, granulocytes, myeloid dendritic cells, and mast cells) and neuronal cells. SIRPs constitute a diverse multigene family of immune receptors encompassing inhibitory, activating, non-signaling and soluble members. CD47, a broadly expressed transmembrane glycoprotein, functions as a cellular ligand for SIRPα and binds to the NH₂-terminal extracellular terminus of SIRPα, i.e., a region of SIRPα referred to as the d1 domain. SIRPα's role has been best documented in respect of its inhibitory role in the phagocytosis of host cells by macrophages and antibody-directed cellular cytotoxicity (ADCC) by neutrophils. In particular, the binding of SIRPα on myeloid cells by CD47 expressed on target cells, generates an inhibitory signal that negatively regulates phagocytosis and ADCC. Agents that bind to either CD47 or to SIRPα and antagonize the CD47: SIRPα interaction act to active macrophage phagocytosis and neutrophil ADCC, particularly towards antibody-opsonized cells (Majeti et al. (2009) Cell. 138(2): 286-99; Chao et al. (2010) Cell. 142(5): 699-713; Zhang et al. (2016) PLoS ONE. 11(4): e15355; and Weiskopf et al. (2013) Science. 341(6141): 88-91). The agents include, but are not limited to, e.g., monoclonal antibodies, soluble CD47, and SIRPα receptor “decoys.” CD47 is also a ligand for SIRPγ, i.e., a gene distinct from SIRPα that is expressed on lymphocytes of unclear function. SIRPβ1 and SIRPβ2 are also distinct genes from SIRPα, and despite their similarity in sequence and structure to SIRPα, they do not naturally bind CD47. However, they can be made to do so through mutation (Hatherley et al. (2008) Molecular Cell. 31(2):266-77). Without being bound by theory, decoy polypeptides comprising a SIRPγ variant, a SIRPβ1 variant, or a SIRPβ2 variant may antagonize the CD47: SIRPα interaction to increase myeloid cell phagocytosis or ADCC. As the SIRPα ectodomain is highly polymorphic between individuals, administration of a recombinant SIRPα therapeutic may increase the likelihood of immunogenicity if it were administered to patients. By contrast, the ectodomains of SIRPγ, SIRP1, and SIRPβ2 are not widely polymorphic, and thus may be less likely or unlikely to induce an immune response in a patient following administration.

### SIRPγ Variants

The amino acid sequence of full-length wild type human SIRPγ (also known as CD172g) is available in the SWISS-PROT database as Q9P1W8. The 387 amino acid sequence of SIRPγ comprises an extracellular domain (ECD) with four potential N-glycosylation sites, a transmembrane domain, and a cytoplasmic sequence. SIRPγ comprises one V-type Ig-like domain comprising a J-like sequence and two C1-type Ig-like domains, within its ECD (Barclay et al. (2006) Nat. Rev. Immunol. 6: 457; van Beek et al. (2005) J. Immunol. 175: 77811 Isoforms that lack one (isoform 2, 2⁷6 aa) or two (isoform 3, 170 aa) membrane-proximal C-type Ig-like domains have been described (Piccio et al. (2005) Blood, 105: 2421).

In some embodiments, the decoy polypeptide comprises SIRPγ variant (e.g., a soluble SIRPγ variant that lacks a transmembrane domain), which variant comprises at least one amino acid substitution relative to a wild type SIRPγ (e.g., relative to the extracellular domain (ECD) of a wild type human SIRPγ), wherein the substitution increases the affinity the SIRPγ variant for CD47 as compared to the affinity of the wild type SIRPγ for CD47. In some embodiments, the at least one substitution is within the d1 domain of the SIRPγ variant. In some embodiments, the at least one substitution is relative to the d1 domain of a wild type SIRPγ (e.g., a wild type human SIRPγ). In some embodiments, the d1 domain comprises amino acids 29-147 of a wild type SIRPγ, e.g., a wild type SIRPγ having the Uniprot accession number Q9P1W8. In some embodiments the at least one substitution is relative to the d1 domain of a wild type SIRPγ set forth in EEELQMIQPE KLLLVTVGKT ATLHCTVTSL LPVGPVLWFR GVGPGRELIY NQKEGHFPRV TTVSDLTKRN NMDFSIRISS ITPADVGTYY CVKFRKGSPE NVEFKSGPGT EMALGAKPS (SEQ ID NO: 1).

In some embodiments, the soluble SIRPγ variant comprises an amino acid sequence that is at least about any one of 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a wild type SIRPγ d1 domain, e.g., of a wild type SIRPγ d1 domain set forth in EEELQMIQPE KLLLVTVGKT ATLHCTVTSL LPVGPVLWFR GVGPGRELIY NQKEGHFPRV TTVSDLTKRN NMDFSIRISS ITPADVGTYY CVKFRKGSPE NVEFKSGPGT EMALGAKPS (SEQ ID NO: 1). In some embodiments, the soluble SIRPγ variant comprises an amino acid sequence that is at least about 90% identical to SEQ ID NO: 1.

In some embodiments, the SIRPγ variant (e.g., soluble SIRPγ variant) comprises one or more amino acid substitutions, deletions, insertions, inversions, and/or modifications relative to SEQ ID NO: 1. In some embodiments, the SIRPγ variant (e.g., soluble SIRPγ variant) comprises one or more unnatural amino acids, one or more D-amino acids, and/or one or more non-proteinogenic amino acids (i.e., amino acids that are not naturally genetically encoded or found in the genetic code).

In some embodiments, the amino acid substitutions, deletions, insertions, inversions, and/or modifications do not substantially reduce the ability of the SIRPγ variant (e.g., soluble SIRPγ variant) to bind CD47, relative to a Wild type SIRPγ. For example, conservative substitutions that do not substantially reduce CD47 binding affinity may be made.

Conservative substitutions are shown in Table 1 under the heading of “conservative substitutions.” More substantial changes are provided in Table 1 under the heading of “exemplary substitutions,” and as further described below in reference to amino acid side chain classes.

TABLE 1
Conservative Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Non-conservative substitutions entail exchanging a member of one of these classes for another class.

In some embodiments, the amino acid substitutions, deletions, insertions, inversions, and/or modifications increase (such as improve) the ability of the SIRPγ variant (e.g., soluble SIRPγ variant) to bind CD47, relative a wild type SIRPγ. Amino acid substitutions, deletions, insertions, inversions, and/or modifications that increase affinity of the SIRPγ variant (e.g., soluble SIRPγ variant) to bind CD47, relative a wild type SIRPγ, may identified by known methods, such as site-directed mutagenesis, crystallization, nuclear magnetic resonance, photoaffinity labeling, or alanine-scanning mutagenesis (Cunningham et al., Science, 244:1081-1085 (1989); Smith et al., J Mol. Biol., 224:899-904 (1992); de Vos et al., Science, 255:306-312 (1992)). The affinity of a SIRPγ variant (e.g., soluble SIRPγ variant) for CD47 may be measured using methods known in the art, such as ELISA, fluorescence activated cell sorting (FACS) analysis, or radioimmunoprecipitation (RIA). Binding of a SIRPγ variant to CD47 can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

In some embodiments, the SIRPγ variant (e.g., soluble SIRPγ variant) comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen or at least eighteen amino acid substitutions. In some embodiments, the amino acid substitutions are at one or more of M6, V27, L30, L31, V33, V36, L37, V42, E47, Q52, K53, E54, H56, L66, T67, V92, S98, and N101, wherein the amino acid positions are relative to the wild-type human SIRPγ d1 domain sequence set forth in SEQ ID NO: 1. In some embodiments, the SIRPγ variant (e.g., soluble SIRPγ variant) comprises a substitution at M6. In some embodiments, the substitution at M6 is M6I, M6L, or M6F. In some embodiments, the SIRPγ variant (e.g., soluble SIRPγ variant) comprises a substitution at V27. In some embodiments, the substitution at V27 is V27F, V27I, or V27L. In some embodiments, the SIRPγ variant (e.g., soluble SIRPγ variant) comprises a substitution at L30. In some embodiments, the substitution at L30 is L30I, L30V, L30H, L30N, or L30D. In some embodiments, the SIRPγ variant (e.g., soluble SIRPγ variant) comprises a substitution at L31. In some embodiments, the substitution at L31 is L31F, L31I, L31V, L31T, or L31S. In some embodiments, the SIRPγ variant (e.g., soluble SIRPγ variant) comprises a substitution at V33. In some embodiments, the substitution at V33 is V33I, V33L, V33P, V33T, or V33A. In some embodiments, the SIRPγ variant (e.g., soluble SIRPγ variant) comprises a substitution at V36. In some embodiments, the substitution at V36 is V36I. In some embodiments, the SIRPγ variant (e.g., soluble SIRPγ variant) comprises a substitution at L37. In some embodiments, the substitution at L37 is L37Q. In some embodiments, the SIRPγ variant (e.g., soluble SIRPγ variant) comprises a substitution at V42. In some embodiments, the substitution is V42A. In some embodiments, the SIRPγ variant (e.g., soluble SIRPγ variant) comprises a substitution at E47. In some embodiments, the substitution at E47 is E47V. In some embodiments, the SIRPγ variant (e.g., soluble SIRPγ variant) comprises a substitution at Q52. In some embodiments, the substitution at Q52 is Q52P, Q52L, Q52V, Q52A or Q52E. In some embodiments, the SIRPγ variant (e.g., soluble SIRPγ variant) comprises a substitution at K53. In some embodiments, the substitution at K53 is K53R. In some embodiments, the SIRPγ variant (e.g., soluble SIRPγ variant) comprises a substitution at E54. In some embodiments, the substitution at E54 is E54D, E54K, E54N, E54Q or E54H. In some embodiments, the SIRPγ variant (e.g., soluble SIRPγ variant) comprises a substitution at H56. In some embodiments, the substitution at H56 is H56P or H56R. In some embodiments, the SIRPγ variant (e.g., soluble SIRPγ variant) comprises a substitution at L66. In some embodiments, the substitution at L66 is L66I, L66V, L66P, L66T, L66A, L66R, L66S or L66G. In some embodiments, the SIRPγ variant (e.g., soluble SIRPγ variant) comprises a substitution at T67. In some embodiments, the substitution at T67 is T67I, T67N, T67F, T67S, T67Y, T67V, T67A or T67D. In some embodiments, the SIRPγ variant (e.g., soluble SIRPγ variant) comprises a substitution at V92. In some embodiments, the substitution at V92 is V92I. In some embodiments, the SIRPγ variant (e.g., soluble SIRPγ variant) comprises a substitution at S98. In some embodiments, the substitution at S98 is S98R, S98N, S98K, S98T, S981 or S98M. In some embodiments, the SIRPγ variant (e.g., soluble SIRPγ variant) comprises a substitution at N101. In some embodiments, the substitution at N101 is N101K, N101D, N101E, N101H or N101Q.

In some embodiments, the decoy polypeptide comprises a SIRPγ variant that comprises the amino acid sequence: EEELQX₁IQPE KLLLVTVGKT ATLHCTX₂TSX₃X₄PX₅GPX₆X₇WFR GX₈GPGRX₉LIY NX₁₀X₁₁X₁₂GX₁₃FPRV TTVSDX₁₄X₁₅KRN NMDFSIRISS ITPADVGTYY CX₁₆KFRKGX₁₇PE X₁₈VEFKSGPGT EMALGAKPS (SEQ ID NO: 2), wherein X₁ is M, I, L or F; X₂ is F, I, L or V; X₃ is L, I, V, H, N or D; X₄ is F, I, L, V, T, or S; X₅ is V, I, L, P, T or A; X₆ is V or I; X₇ is L or Q; X₈ is V or A; X₉ is E or V; X₁₀ is Q, P, L, V, A or E; X₁₁ is K or R; X₁₂ is E, D, K, N, Q or H; X₁₃ is H, P or R; X₁₄ is L, I, V, P, T, A, R, S or G; X₁₅ is T, I, N, F, S, Y, V, A or D; X₁₆ is V or I; X₁₇ is S, R, N, K, T, I or M; and X₁₈ is N, K, D, E, H or Q.

In some embodiments, the decoy polypeptide comprises a SIRPγ variant that comprises an amino acid sequence set forth in any one of SEQ ID NOs: 3-24 and 42.

The amino acid sequences of SEQ ID NOs: 3-24 and 42 are provided below:

(SEQ ID NO: 3)
EEELQIIQPE KLLLVTVGKT ATLHCTITSL FPVGPVLWFR
GVGPGRVLIY NQRQGPFPRV TTVSDTTKRN NMDFSIRISS
ITPADVGTYY CIKFRKGSPE NVEFKSGPGT EMALGAKPS
(SEQ ID NO: 4)
EEELQIIQPE KLLLVTVGKT ATLHCTITSL FPVGPIQWFR
GVGPGRVLIY NQRDGPFPRV TTVSDGTKRN NMDFSIRISS
ITPADVGTYY CVKFRKGTPE DVEFKSGPGT EMALGAKPS
(SEQ ID NO: 5)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPS
(SEQ ID NO: 6)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPS
(SEQ ID NO: 7)
EEELQIIQPE KLLLVTVGKT ATLHCTITSL FPVGPIQWFR
GAGPGRVLIY NQRDGPFPRV TTVSDGTKRN NMDFSIRISS
ITPADVGTYY CIKFRKGTPE DVEFKSGPGT EMALGAKPS
(SEQ ID NO: 8)
EEELQIIQPE KLLLVTVGKT ATLHCTITSH FPVGPIQWFR
GVGPGRVLIY NQKDGHFPRV TTVSDGTKRN NMDFSIRISS
ITPADVGTYY CVKFRKGSPE DVEFKSGPGT EMALGAKPS
(SEQ ID NO: 9)
EEELQIIQPD KSVLVAAGET ATLRCTITSL FPVGPIQWFR
GAGPGRVLIY NQRDGPFPRV TTVSDGTKRN NMDFSIRISS
ITPADVGTYY CVKFRKGTPE DVEFKSGPGT EMALGAKPS
(SEQ ID NO: 10)
EEELQIIQPE KLLLVTVGKT ATLHCTITSL FPVGPVLWFR
GVGPGRVLIY NQRQGPFPRV TTVSDTTKRN NMDFSIRISS
ITPADVGTYY CVKFRKGTPE DVEFKSGPGT EMALGAKPS
(SEQ ID NO: 11)
EEELQIIQPE KLLLVTVGKT ATLHCTITSL FPVGPIQWFR
GVGPGRELIY NAREGRFPRV TTVSDLTKRN NMDFSIRISS
ITPADVGTYY CVKFRKGSPE DVEFKSGPGT EMALGAKPS
(SEQ ID NO: 12)
EEELQIIQPD KSVLVAAGET ATLRCTITSL FPVGPIQWFR
GAGPGRVLIY NQRQGPFPRV TTVSDTTKRN NMDFSIRIGN
ITPADAGTYY CIKFRKGSPD DVEFKSGAGT ELSVRAKPS
(SEQ ID NO: 13)
EEELQIIQPE KLLLVTVGKT ATLHCTITSL FPVGPIQWFR
GVGPGRVLIY NQREGPFPRV TTVSDGTKRN NMDFSIRISS
ITPADVGTYY CVKFRKGSPE DVEFKSGPGT EMALGAKPS
(SEQ ID NO: 14)
EEELQIIQPD KSVLVAAGET ATLRCTITSL FPVGPIQWFR
GAGPGRVLIY NQRDGPFPRV TTVSDGTKRN NMDFSIRISS
ITPADVGTYY CIKFRKGIPE DVEFKSGPGT EMALGAKPS
(SEQ ID NO: 15)
EEELQIIQPD KSVLVAAGET ATLRCTITSL FPVGPIQWFR
GAGPGRVLIY NQRDGPFPRV TTVSDGTKRN NMDFSIRISS
ITPADVGTYY CIKFRKGIPE DVEFKSGPGTXWH,
wherein X is A, R, N, D, C, Q, E, G, H, I, L,
K, M, F, P, S, T, W, Y, or V
(SEQ ID NO: 16)
EEELQIIQPD KSVLVAAGET ATLRCTITSL FPVGPIQWFR
GAGPGRVLIY NQKDGPFPRV TTVSDGTKRN NMDFSIRISS
ITPADVGTYY CIKFRKGTPE DVEFKSGPGT EMALGAKPS
(SEQ ID NO: 17)
EEELQIIQPE KLLLVTVGKT ATLHCTITSL LPVGPIQWFR
GVGPGRELIY NQRDGPFPRV TTVSDGTKRN NMDFSIRISS
ITPADVGTYY CVKFRKGTPE DVEFKSGPGT EMALGAKPS
(SEQ ID NO: 18)
EEELQIIQPE KLLLVTVGKT ATLHCTLTSL LPVGPILWFR
GVGPGRVLIY NQRDGPFPRV TTVSDGTKRN NMDFSIRISS
ITPADVGTYY CVKFRKGNPE DVEFKSGPGT EMALGAKPS
(SEQ ID NO: 19)
EEELQLIQPE KLLLVTVGKT ATLHCTITSL FPPGPIQWFR
GVGPGRVLIY NQKDGPFPRV TTVSDGTKRN NMDFSIRISS
ITPADVGTYY CVKFRKGIPE DVEFKSGPGT EMALGAKPS
(SEQ ID NO: 20)
EEELQIIQPE KLLLVTVGKT ATLRCTITSL FPVGPIQWFR
GAGPGRVLIY NQRDGPFPRV TTVSDGTKRN NMDFSIRISS
ITPADVGTYY CIKFRKGIPE DVEFKSGPGT EMALGAKPS
(SEQ ID NO: 21)
EEELQIIQPE KLLLVTVGKT ATLHCTITSL FPIGPILWFR
GVGPGRVLIY NQKDGPFPRVT TVSDGTKRN NMDFSIRISS
ITPADVGTYY CVKFRKGSPE DVEFKSGPGT EMALGAKPS
(SEQ ID NO: 22)
EEELQMIQPE KLLLVTVGKT ATLHCTITSL FPVGPIQWFR
GAGPGRVLIY NQRDGPFPRV TTVSDGTKRN NMDFSIRISS
ITPADVGTYY CIKFRKGIPE DVEFKSGPGT EMALGAKPS
(SEQ ID NO: 23)
EEELQIIQPD KSVLVAAGET ATLRCTITSL FPVGPIQWFR
GVGPGRVLIY NQKDGPFPRV TTVSDGTKRN NMDFSIRISS
ITPADVGTYY CIKFRKGIPE DVEFKSGPGT EMALGAKPS
(SEQ ID NO: 24)
EEELQIIQPD KSVLVAAGET ATLRCTITSL FPVGPIQWFR
GAGPGRVLIY NQKDGPFPRV TTVSDGTKRN NMDFSIRISS
ITPADVGTYY CIKFRKGTPE DVEFKSGPGT EMALXAKPS
(SEQ ID NO: 42)
EEELQMIQPE KLLLVTVGKT ATLHCTVTSL LPVGPVLWFR
GVGPGRELIY NQKEGHFPRV TTVSDLTKRN NMDFSIRISS
ITPADVGTYY CVKFRKGSPE NVEFKSGPGT EMALGAKPS

In some embodiments, the SIRPγ variant is more resistant to proteolytic cleavage as compared to a wild type SIRPγ (e.g., a wild type human SIRPγ). In some embodiments, the SIRPγ variant has a longer circulating half-life as compared to a wild type SIRPγ (e.g., a wild type human SIRPγ). In some embodiments, the SIRPγ variant is more resistant to oxidation as compared to a wild type SIRPγ (e.g., a wild type human SIRPγ).

### SIRPβ1 Variants

The amino acid sequence of human SIRPβ1 (also known as Signal Regulatory Protein Beta 1, CD172b, and SIRP beta 1 isoform 1) is available in the SWISS-PROT database as 000241. SIRPβ1 is a transmembrane protein that has three Ig-like domains in its extracellular region and a short cytoplasmic tail that lacks cytoplasmic sequence motifs capable of recruiting SHP-2 and SHP-1. SIRPβ1 does not bind CD47 and lacks cytoplasmic immunoreceptor tyrosine-based inhibition motifs (ITIMs). The hydrophobic transmembrane domain of SIRPβ1 contains a single basic lysine residue, which may facilitate interaction with signaling adaptor protein DAP12. Multiple transcript variants encoding three different isoforms of SIRPβ1 have been identified.

In some embodiments, the decoy polypeptide comprises a soluble SIRPβ1 variant (i.e., SIRPβ1 variant that lacks a transmembrane domain), which variant comprises at least one amino acid substitution relative to a wild type SIRPβ1 (e.g., relative to the extracellular domain (ECD) of a wild type human SIRPβ1), wherein the substitution increases the affinity the SIRPβ1 variant for CD47 as compared to the affinity of the wild type SIRPβ1 for CD47. In some embodiments, the at least one substitution is within the d1 domain of the SIRPβ1 variant. In some embodiments, the at least one substitution is relative to the d1 domain of a wild type SIRPβ1 (e.g., a wild type human SIRP1). In some embodiments, the d1 domain comprises amino acids 30-148 of a wild type SIRPβ1, e.g., a wild type SIRPβ1 having the Uniprot accession number 000241. In some embodiments the at least one substitution is relative to the d1 domain of a wild type SIRPβ1 set forth in EDELQVIQPE KSVSVAAGES ATLRCAMTSL IPVGPIMWFR GAGAGRELIY NQKEGHFPRV TTVSELTKRN NLDFSISISN ITPADAGTYY CVKFRKGSPD DVEFKSGAGT ELSVRAKPS (SEQ ID NO: 25).

In some embodiments, the soluble SIRPβ1 variant comprises an amino acid sequence that is at least about any one of 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a wild type SIRP1I d1 domain set forth in: EDELQVIQPE KSVSVAAGES ATLRCAMTSL IPVGPIMWFR GAGAGRELIY NQKEGHFPRV TTVSELTKRN NLDFSISISN ITPADAGTYY CVKFRKGSPD DVEFKSGAGT ELSVRAKPS (SEQ ID NO: 25). In some embodiments, the soluble SIRPβ1 variant comprises an amino acid sequence that is at least about 90% identical to SEQ ID NO: 25.

In some embodiments, the soluble SIRPβ1 variant comprises one or more amino acid substitutions, deletions, insertions, inversions, and/or modifications relative to SEQ ID NO: 25. In some embodiments, the soluble SIRPβ1 variant comprises one or more unnatural amino acids, one or more D-amino acids, and/or one or more non-proteinogenic amino acids (i.e., amino acids that are not naturally genetically encoded or found in the genetic code). Conservative substitutions are shown in Table 1 above under the heading of “conservative substitutions.” More substantial changes are provided in Table 1 above under the heading of “exemplary substitutions,” and as further described below in reference to amino acid side chain classes. As discussed above, non-conservative substitutions entail exchanging a member of one of these classes for another class.

In some embodiments, the amino acid substitutions, deletions, insertions, inversions, and/or modifications increase (such as improve) the ability of the soluble SIRPβ1 variant to bind CD47, relative a wild type SIRPβ1. Amino acid substitutions, deletions, insertions, inversions, and/or modifications that increase affinity of the soluble SIRPβ1 variant to bind CD47, relative a wild type SIRPβ1, may identified by known methods, e.g., methods described elsewhere herein. The affinity of a soluble SIRPβ1 variant for CD47 may be measured using methods known in the art, e.g., methods described elsewhere herein.

In some embodiments, the soluble SIRPβ1 variant that comprises at least one, at least two, at least three, at least four, at least five, at least six, at least seven, at least eight, at least nine, at least ten, or at least eleven amino acid substitutions at one or more of V6, M27, 131, M37, E47, K53, E54, H56, L66, N80, or V92, wherein the amino acid positions are relative to a wild-type human SIRPβ1 d1 domain sequence set forth in SEQ ID NO: 25. In some embodiments, the soluble SIRPβ1 variant comprises an amino acid substitution at V6. In some embodiments, the substitution at V6 is V6I. In some embodiments, the soluble SIRPβ1 variant comprises an amino acid substitution at M27. In some embodiments, the substitution at M27 is M27I. In some embodiments, the soluble SIRPβ1 variant comprises an amino acid substitution at I31. In some embodiments, the substitution at I31 is I31F. In some embodiments, the soluble SIRP-SIRPβ1 variant comprises an amino acid substitution at M37. In some embodiments, the substitution at M37 is M37Q. In some embodiments, the soluble SIRPβ1 variant comprises an amino acid substitution at E47. In some embodiments, the substitution at E47 is E47V. In some embodiments, the soluble SIRPβ1 variant comprises an amino acid substitution at K53. In some embodiments, the substitution at K53 is K53R. In some embodiments, the soluble SIRPβ1 variant comprises an amino acid substitution at E54. In some embodiments, the substitution at E54 is E54Q. In some embodiments, the soluble SIRPβ1 variant comprises an amino acid substitution at H56. In some embodiments, the substitution at H56 is H56P. In some embodiments, the soluble SIRPβ1 variant comprises an amino acid substitution at L66. In some embodiments, the substitution at L66 is L66T. In some embodiments, the soluble SIRPβ1 variant comprises an amino acid substitution at N80. In some embodiments, the substitution at N80 is N80A, N80C, N80D, N80E, N80F, N80G, N80H, N80I, N80K, N80L, N80M, N80P, N80Q, N80R, N80S, N80T, N80V, N80W, or N80Y. In some embodiments, the substitution at N80 (such as any of the preceding) minimizes or abrogates partial glycosylation of the soluble SIRPβ1 variant. In some embodiments, the substitution at N80 (such as any of the preceding) confers a functional benefit of increasing the homogeneity associated with a soluble SIRPβ1 variant. In some embodiments, the substitution at N80 (such as any of the preceding) removes a glycosylation site in a soluble SIRPβ1 variant, thereby allowing the production of a more uniform protein therapeutic following manufacture. In some embodiments, the soluble SIRPβ1 variant comprises an amino acid substitution at V92. In some embodiments, the substitution at V92 is V92I.

In some embodiments, the SIRPβ1 variant comprises the amino acid sequence EDELQX₁IQPE KSVSVAAGES ATLRCAX₂TSL X₃PVGPIX₄WFR GAGAGRX₅LIY NQX₆X₇GX₈FPRV TTVSEX₉TKRN NLDFSISISX₁₀ITPADAGTYY CX₁₁KFRKGSPD DVEFKSGAGT ELSVRAKPS (SEQ ID NO: 45) wherein X₁ is V or I; X₂ is M or I; X₃ is I or F; X₄ is M or Q; X₅ is E or V; X₁₆ is K or R; X₇ is E or Q; X₈ is H or P; X₉ is L or T; X₁₀ is any amino acid; and X₁₁ is V or I. In some embodiments, X₁₀ is any amino acid other than N. In some embodiments, X₁₀ is A. In some embodiments, the decoy polypeptide comprises a SIRPβ1 variant that comprises an amino acid sequence set forth in EDELQIIQPE KSVSVAAGES ATLRCAITSL FPVGPIQWFR GAGAGRVLIY NQRQGPFPRV TTVSETTKRN NLDFSISISN ITPADAGTYY CIKFRKGSPD DVEFKSGAGT ELSVRAKPS (SEQ ID NO: 26). In some embodiments, the decoy polypeptide comprises a SIRPβ1 variant that comprises an amino acid sequence set forth in EDELQIIQPE KSVSVAAGES ATLRCAITSL FPVGPIQWFR GAGAGRVLIY NQRQGPFPRV TTVSETTKRN NLDFSISISA ITPADAGTYY CIKFRKGSPD DVEFKSGAGT ELSVRAKPS (SEQ ID NO: 88). In some embodiments, the soluble SIRPβ1 variant comprises an amino acid sequence that is least about any one of 80%, 85%, 90%, 91%, 92%, 93%, 94%, 95%, 96%, 97%, 98%, or 99% identical to SEQ ID NO: 26 or SEQ ID NO: 88.

In some embodiments, the SIRPβ1 variant is more resistant to proteolytic cleavage as compared to a wild type SIRPβ1 (e.g., a wild type human SIRP1). In some embodiments, the SIRPβ1 variant has a longer circulating half-life as compared to a wild type SIRPβ1 (e.g., a wild type human SIRP1). In some embodiments, the v variant is more resistant to oxidation as compared to a wild type SIRPβ1 (e.g., a wild type human SIRPβ1).

### SIRPβ2 Variants

The amino acid sequence of SIRPβ2 (also known as Signal Regulatory Protein Beta 2, PTPN1L, and SIRP beta 1 isoform 3) is available in the SWISS-PROT database as Q5TFQ8. The amino acid sequence of SIRPβ2 is highly homologous to that of SIRPβ1. However, SIRPβ2 lacks both cytoplasmic ITIMs and the transmembrane lysine required for association with DAP12. Alternatively spliced transcript variants encoding different isoforms of SIRPβ2 have been identified.

In some embodiments, the decoy polypeptide comprises a soluble SIRPβ2 variant (i.e., SIRPβ2 variant that lacks a transmembrane domain), which variant comprises at least one amino acid substitution relative to a wild type SIRPβ2 (e.g., relative to the extracellular domain (ECD) of a wild type human SIRPβ2), wherein the substitution increases the affinity the SIRPβ2 variant for CD47 as compared to the affinity of the wild type SIRPβ2 for CD47. In some embodiments, the at least one substitution is within the d1 domain of the SIRPβ2 variant. In some embodiments, the at least one substitution is relative to the d1 domain of a wild type SIRPβ2 (e.g., a wild type human SIRPβ2). In some embodiments, the d1 domain comprises amino acids 30-148 of a wild type SIRPβ2, e.g. a wildtype SIRPβ2 having the Uniprot accession number Q5TFQ8. In some embodiments the at least one substitution is relative to the d1 domain of a wild type SIRPβ2 set forth in EEELQVIQPD KSISVAAGES ATLHCTVTSL IPVGPIQWFR GAGPGRELIY NQKEGHFPRV TTVSDLTKRN NMDFSIRISN ITPADAGTYY CVKFRKGSPD HVEFKSGAGT ELSVRAKPS (SEQ ID NO: 27).

In some embodiments, the soluble SIRPβ2 variant comprises an amino acid sequence that is at least about any one of 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a wild type SIRPβ2 d1 domain set forth in: EEELQVIQPD KSISVAAGES ATLHCTVTSL IPVGPIQWFR GAGPGRELIY NQKEGHFPRV TTVSDLTKRN NMDFSIRISN ITPADAGTYY CVKFRKGSPD HVEFKSGAGT ELSVRAKPS (SEQ ID NO: 277). In some embodiments, the soluble SIRPβ2 variant comprises an amino acid sequence that is at least about 90% identical to SEQ ID NO: 27.

In some embodiments, the soluble SIRPβ2 variant comprises one or more amino acid substitutions, deletions, insertions, inversions, and/or modifications relative to SEQ ID NO: 27. In some embodiments, the soluble SIRPβ2 variant comprises one or more unnatural amino acids, one or more D-amino acids, and/or one or more non-proteinogenic amino acids (i.e., amino acids that are not naturally genetically encoded or found in the genetic code). Conservative substitutions are shown in Table 1 above under the heading of “conservative substitutions.” More substantial changes are provided in Table 1 above under the heading of “exemplary substitutions,” and as further described below in reference to amino acid side chain classes. As discussed above, non-conservative substitutions entail exchanging a member of one of these classes for another class.

In some embodiments, the amino acid substitutions, deletions, insertions, inversions, and/or modifications increase the ability of the soluble SIRPβ2 variant to bind CD47, relative a wild type SIRPβ2. Amino acid substitutions, deletions, insertions, inversions, and/or modifications that increase affinity of the soluble SIRPβ2 variant to bind CD47, relative a wild type SIRPβ2, may identified by known methods, as discussed elsewhere herein. The affinity of a SIRPβ2 variant for CD47 may be measured using methods known in the art, as discussed elsewhere herein.

In some embodiments, the soluble SIRPβ2 variant that comprises at least 1, at least 2, at least 3, at least 4, at least 5, at least 6, at least 7, at least 8, at least 9, at least 10, or at least 11 amino acid substitutions at one or more of V6, V27, 131, E47, K53, E54, H56, L66, N80, V92 or H101, wherein the amino acid positions are relative to a wild-type human SIRPβ2 d1 domain sequence set forth in SEQ ID NO: 27. In some embodiments, the soluble SIRPβ2 variant comprises an amino acid substitution at V6. In some embodiments, the substitution at V6 is V6I. In some embodiments, the soluble SIRPβ2 variant comprises an amino acid substitution at V27. In some embodiments, the substitution at V27 is V27I. In some embodiments, the soluble SIRPβ2 variant comprises an amino acid substitution at I31. In some embodiments, the substitution at I31 is I31F. In some embodiments, the soluble SIRPβ2 variant comprises an amino acid substitution at E47. In some embodiments, the substitution at E47 is E47V. In some embodiments, the soluble SIRPβ2 variant comprises an amino acid substitution at K53. In some embodiments, the substitution at K53 is K53R. In some embodiments, the soluble SIRPβ2 variant comprises an amino acid substitution at E54. In some embodiments, the substitution at E54 is E54Q. In some embodiments, the soluble SIRPβ2 variant comprises an amino acid substitution at H56. In some embodiments, the substitution at H56 is H56P. In some embodiments, the soluble SIRPβ2 variant comprises an amino acid substitution at L66. In some embodiments, the substitution at L66 is L66T. In some embodiments, the soluble SIRPβ2 variant comprises an amino acid substitution at N80. In some embodiments, the substitution at N80 is N80A, N80C, N80D, N80E, N80F, N80G, N80H, N80I, N80K, N80L, N80M, N80P, N80Q, N80R, N80S, N80T, N80V, N80W, or N80Y. In some embodiments, the substitution at N80 (such as any of the preceding) minimizes or abrogates partial glycosylation of the soluble SIRPβ2 variant. In some embodiments, the substitution at N80 (such as any of the preceding) confers a functional benefit of increasing the homogeneity associated with a soluble SIRPβ2 variant. In some embodiments, the substitution at N80 (such as any of the preceding) removes a glycosylation site in a soluble SIRPβ2 variant, thereby allowing the production of a more uniform protein therapeutic following manufacture. In some embodiments, the soluble SIRPβ2 variant comprises an amino acid substitution at V92. In some embodiments, the substitution at V92 is V92I. In some embodiments, the soluble SIRPβ2 variant comprises an amino acid substitution at H110. In some embodiments, the substitution at H101 is H101D.

In some embodiments, the soluble SIRPβ2 variant comprises the amino acid sequence EEELQX₁IQPD KSISVAAGES ATLHCTX₂TSL X₃PVGPIQWFR GAGPGRX₄LIY NQX₅X₆GX₇FPRV TTVSDX₈TKRN NMDFSIRISX₁₀ ITPADAGTYY CX₉KFRKGSPD X₁₁VEFKSGAGT ELSVRAKPS (SEQ ID NO: 46) wherein X₁ is V or I; X₂ is V or I; X₃ is I or F; X₄ is E or V; X₅ is K or R; X₆ is E or Q; X₇ is H or P; X₈ is L or T; X₉ is V or I; X₁₀ is any amino acid; and X₁₁ is H or D. In some embodiments, X₁₀ is any amino acid other than N. In some embodiments, X₁₀ is A.

In some embodiments, the soluble SIRPβ2 variant that comprises the amino acid sequence EEELQIIQPD KSISVAAGES ATLHCTITSL FPVGPIQWFR GAGPGRVLIY NQRQGPFPRV TTVSDTTKRN NMDFSIRISN ITPADAGTYY CIKFRKGSPD DVEFKSGAGT ELSVRAKPS (SEQ ID NO: 28). In some embodiments, the soluble SIRPβ2 variant that comprises the amino acid sequence EEELQIIQPD KSISVAAGES ATLHCTITSL FPVGPIQWFR GAGPGRVLIY NQRQGPFPRV TTVSDTTKRN NMDFSIRISA ITPADAGTYY CIKFRKGSPD DVEFKSGAGT ELSVRAKPS (SEQ ID NO: 89). In some embodiments, the soluble SIRPβ2 variant comprises an amino acid sequence that is at least about any one of 80%, 85%, 90%, 91%, 92%, 93%, 94%, 95%, 96%, 97%, 98%, or 99% identical to SEQ ID NO: 28 or SEQ ID NO: 89.

Generating SIRPγ Variants, SIRP/Variants, and/or SIRPβ2 Variants

A variety of well-known methods can be used to generate SIRPγ variants, SIRPβ2 variants, and/or SIRPβ2 variants. As one non-limiting example, mutagenesis can be performed (beginning with a wild type SIRPγ (or the extracellular domain (ECD) thereof), a wild type SIRPβ1 (or the ECD thereof), or a wild type SIRPβ2 polypeptide (or the ECD thereof)) to generate collections of SIRPγ variants, SIRPβ1 variants, or SIRPβ2 variants. Mutagenesis can be targeted to produce changes at particular amino acids, or mutagenesis can be random. In another non-limiting example, SIRPγ variants, SIRPβ2 variants, and/or SIRPβ2 variants can be generated via gene synthesis. The SIRPγ variants, SIRPβ1 variants, or SIRPβ2 variants generated using methods known in the art can then be screened for their ability to bind a CD47 protein. For example, a CD47 protein (or a variant of a CD47 protein, e.g., a version lacking a transmembrane domain) can be labeled (e.g., with a direct label such as a radioisotope, a fluorescent moiety, etc.; or with an indirect label such as an antigen, an affinity tag, biotin, etc.) and used to contact the candidate SIRPγ variant, SIRPβ1 variant or SIRPβ2 variant (e.g., where the candidate SIRPγ variant, SIRPβ1 variant, or SIRPβ2 variant can be attached to a solid surface or displayed on the membrane of a cell, e.g., a yeast cell). By varying the concentration of CD47 used, one can identify high-affinity SIRPγ variant, SIRPβ1 variants, or SIRPβ2 variants from among the candidates.

### Human Fc Variants

The Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Engineering the Fc region of a therapeutic monoclonal antibody or Fc fusion protein allows the generation of molecules that are better suited to the pharmacology activity required of them. The half-life of an IgG depends on its pH-dependent binding to the neonatal receptor FcRn. FcRn, which is expressed on the surface of endothelial cells, binds the IgG in a pH-dependent manner and protects it from degradation.

A “wild-type Fc region” possesses the effector functions of a native-sequence Fc region, in particular for the purposes of the present invention interacting with one or more of the Fc receptors such as FcγRI (also known as CD64); FcγRIIA (also known as CD32a), FcγRIIB (also known as CD32b); FcγRIIC (also known as CD32c), FcγRIIIA (also known as CD16a); FcγRIIIB (also known as CD16b) receptors; and can be assessed using various assays as disclosed, for example, in definitions herein. A “dead” Fc is one that has been mutagenized to retain activity with respect to, for example, prolonging serum half-life through interaction with FcRn, but which has reduced or absent binding to one or more other Fc receptor(s), including without limitation a human FcγR as listed above.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

In some embodiments, a decoy polypeptide provided herein comprises a variant Fc region or an engineered Fc region. A “variant Fc region” or “engineered Fc region” refers to an Fc region that comprises an amino acid sequence that differs from that of a native-sequence Fc region by virtue of, e.g., at least one amino acid modification, or, e.g., one or more amino acid substitution(s). In some embodiments, the decoy polypeptide comprises a variant Fc region that has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. In some embodiments, the decoy polypeptide comprises a variant Fc region having, e.g., at least about 80% homology with a native sequence Fc region and/or with an Fc region of a parent polypeptide, at least about 85% homology therewith, least about 90% homology therewith, at least about 95% homology therewith, at least about 96% homology therewith, at least about 97% homology therewith, at least about 98% homology therewith, or at least about 96% homology therewith, including any range in between these values.

Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

In some embodiments, the decoy polypeptide comprises a dead Fc. For example, variant Fc sequences for a “dead Fc” may include three amino acid substitutions in the CH2 region to reduce FcγRI binding at EU index positions 234, 235, and 237 (see Duncan et al., (1988) Nature 332:563). Two amino acid substitutions in the complement CIq binding site at EU index positions 330 and 331 reduce complement fixation (see Tao et al., J. Exp. Med. 178:661 (1993) and Canfield and Morrison, J. Exp. Med. 173: 1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). In another, non-limiting example, binding of IgG Fcs to the FcγRs or C1q depends on residues located in the hinge region and the CH2 domain. Two regions of the CH2 domain are critical for FcγRs and C1q binding, and have unique sequences in IgG2 and IgG4. Substitutions into human IgG1 or IgG2 residues at EU positions 233-236 and IgG4 residues at EU positions 327, 330 and 331 have been shown to greatly reduce ADCC and CDC. Numerous mutations have been made in the CH2 domain of human IgG1.

In some embodiments, a decoy polypeptide comprises a human Fc variant that comprises an amino acid substitution at L234A, L235A, and/or G237A (wherein numbering is according to the EU index of Kabat). In some embodiments, a decoy polypeptide comprises a human Fc variant that comprises amino acid substitutions at L234A, L235A, and G237A (wherein numbering is according to the EU index of Kabat). This combination of mutations largely eliminates FcγR and complement effector functions (see, for example, US20100266505).

In some embodiments, the decoy polypeptide comprises a human Fc variant that has been modified by the choice of expression host and/or enzymatic treatment of amino acid substitutions to have reduced glycosylation and binding to FcγR, relative to the native protein. Mutations that reduce binding to FcγR include, without limitation, modification of the glycosylation at EU position N297 of the Fc domain, which is known to be required for optimal FcR interaction. For example known amino acid substitutions include, but are not limited to, e.g., N297A, N297Q, N297D, N297H, and N297G. Such changes result in the loss of a glycosylation site on the Fc domain. Enzymatically deglycosylated Fc domains, recombinantly expressed antibodies in the presence of a glycosylation inhibitor, and the expression of Fc domains in bacteria have a similar loss of glycosylation and consequent binding to FcγRs.

In some embodiments, the decoy polypeptide comprises a human Fc variant comprising mutations that significantly reduce FcγR binding. In some embodiments, the decoy polypeptide comprises a human Fc variant comprising LALA mutations, i.e., L234A/L235A (wherein numbering is according to the EU index of Kabat). In some embodiments, the decoy polypeptide comprises one or more of E233P, L234V, L235A, delG236, A327G, A330S, and P331S mutations, (wherein numbering is according to the EU index of Kabat). In some embodiments, the decoy polypeptide comprises E233P, L234V, L235A, delG236, A327G, A330S, and P331S mutations, (wherein numbering is according to the EU index of Kabat). See, for example, Armour et al. (1999) Eur J Immunol. 29(8):2613-24. In some embodiments, the decoy polypeptide comprises K322A, L234A and L235A mutations (wherein numbering is according to the EU index of Kabat) are sufficient to almost completely abolish FcγR and C1q binding. In some embodiments, the decoy polypeptide comprises L234F, L235E, and P331S substitutions (wherein numbering is according to the EU index of Kabat).

Decoy polypeptides comprising other human Fc variants are contemplated, including, without limitation, human Fc variants comprising amino acid substitution(s) and/or deletion(s) that render the variant incapable of forming disulfide bonds, human Fc variants in which residue(s) at the N-terminus have been deleted, and human Fc variants comprising additional methionine residue(s) at the N-terminus. In some embodiments, the decoy polypeptide comprises a human Fc variant that comprises native sugar chains, increased sugar chains compared to a native form, or decreased sugar chains compared to the native form. In some embodiments, the decoy polypeptide comprises an aglycosylated or deglycosylated human Fc variant. The increase, decrease, removal or other modification of the sugar chains may be achieved by methods common in the art, such as a chemical method, an enzymatic method or by expressing it in a genetically engineered production cell line. Such cell lines can include microorganisms, e.g. Pichia Pastoris, and mammalians cell line, e.g. CHO cells, that naturally express glycosylating enzymes. Further, microorganisms or cells can be engineered to express glycosylating enzymes, or can be rendered unable to express glycosylation enzymes (see e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al., BMC Biotechnolog 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies or fusion polypeptides comprising an Fc domain expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See e.g., WO 07/117505). The presence of particular glycosylation moieties has been shown to alter the effector function of immunoglobulins and fusion polypeptides comprising an Fc domain. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (see e.g., Kaneko, et al., Science 313:760 (2006)), whereas removal of fucose from the IgG leads to enhanced ADCC activity (see e.g., Shoj-Hosaka, et al., J. Biochem., 140:777 (2006)).

In some embodiments, the decoy polypeptide comprises a human Fc variant selected from the group consisting of (i) a human IgG1 Fc variant comprising L234A, L235A, G237A, and N297A substitutions (wherein numbering is according to the EU index of Kabat); (ii) a human IgG2 Fc variant comprising A330S, P331S and N297A substitutions (wherein numbering is according to the EU index of Kabat); or (iii) a human IgG4 Fc variant comprising S228P, E233P, F234V, L235A, delG236, and N297A substitutions (wherein numbering is according to the EU index of Kabat).

In some embodiments, the decoy polypeptide comprises a human IgG1 Fc variant comprising L234A, L235A, G237A, or N297A substitutions (wherein numbering is according to the EU index of Kabat). In some embodiments, the decoy polypeptide comprises a human IgG1 Fc variant comprising two or more of L234A, L235A, G237A, or N297A substitutions (wherein numbering is according to the EU index of Kabat). In some embodiments, the decoy polypeptide comprises a human IgG1 Fc variant comprising L234A, L235A, G237A, and N297A substitutions (wherein numbering is according to the EU index of Kabat).

In some embodiments, the decoy polypeptide comprises a human IgG1 Fc variant comprising a D265 substitution (wherein numbering is according to the EU index of Kabat). In some embodiments, the decoy polypeptide comprises a human IgG1 Fc variant comprising L234A, L235A, G237A, D265, and N297A substitutions (wherein numbering is according to the EU index of Kabat).

In some embodiments, the human Fc variant exhibits ablated or reduced binding to an Fcγ receptor compared to a wild-type human IgG1 Fc. In some embodiments, the human Fc variant exhibits ablated or reduced binding to CD16a, CD32a, CD32b, CD32c, and CD64 Fcγ receptors compared to a wild-type human IgG1 Fc. In some embodiments, the human Fc variant exhibits ablated or reduced binding to C1q compared to a wild-type human IgG1 Fc.

In some embodiments, the decoy polypeptide comprises a human IgG2 Fc variant comprising A330S, P331S or N297A substitutions (wherein numbering is according to the EU index of Kabat). In some embodiments, the decoy polypeptide comprises a human IgG2 Fc variant comprising two or more of A330S, P331S and N297A substitutions (wherein numbering is according to the EU index of Kabat). In some embodiments, the decoy polypeptide comprises a human IgG2 Fc variant comprising A330S, P331S and N297A substitutions (wherein numbering is according to the EU index of Kabat). In some embodiments, the human Fc variant exhibits ablated or reduced binding to an Fcγ receptor compared to a wild-type human IgG2 Fc. In some embodiments, the human Fc variant exhibits ablated or reduced binding to CD16a, CD32a, CD32b, CD32c, and CD64 Fcγ receptors compared to a wild-type human IgG2 Fc. In some embodiments, the human Fc variant exhibits ablated or reduced binding to C1q compared to a wild-type human IgG2 Fc.

In some embodiments, the decoy polypeptide comprises a human IgG4 Fc variant comprising an S228P substitution (wherein numbering is according to the EU index of Kabat). In some embodiments, the decoy polypeptide comprises a human IgG4 Fc variant comprising S228P and L235E substitutions (wherein numbering is according to the EU index of Kabat). In some embodiments, the decoy polypeptide comprises a human IgG4 Fc variant comprising S228P, E233P, F234V, L235A, delG236, or N297A mutations (wherein numbering is according to the EU index of Kabat). In some embodiments, the decoy polypeptide comprises a human IgG4 Fc variant comprising two or more of S228P, E233P, F234V, L235A, delG236, and N297A mutations (wherein numbering is according to the EU index of Kabat). In some embodiments, the decoy polypeptide comprises a human IgG4 Fc variant comprising S228P, E233P, F234V, L235A, delG236, and N297A mutations (wherein numbering is according to the EU index of Kabat). In some embodiments, the human Fc variant exhibits ablated or reduced binding to a Fcγ receptor compared to a wild-type human IgG4 Fc. In some embodiments, the human Fc variant exhibits ablated or reduced binding to CD16a and CD32b Fcγ receptors compared to a wild-type human IgG4 Fc.

In some embodiments, the human Fc variant comprises an amino acid sequence set forth in any one of SEQ ID NOs: 48-51, 53-56, 93-96, and 98-101 below.

(SEQ ID NO: 47)
DKTHTCPPCP APELLGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPG
(SEQ ID NO: 48)
DKTHTCPPCP APELLGGPSV FLFPPKPKDT LMISRTPEVT
CVVVAVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPG
(SEQ ID NO: 49)
DKTHTCPPCP APEAAGAPSV FLFPPKPKDT LMISRTPEVT
CVVVDVSHED PEVKFNWYVD GVEVHNAKTK PREEQYASTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPG
(SEQ ID NO: 50)
DKTHTCPPCP APEAAGAPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPG
(SEQ ID NO: 51)
DKTHTCPPCP APELLGGPSV FLFPPKPKDT LMISRTPEVT
CVVVDVSHEDP EVKFNWYVD GVEVHNAKTK PREEQYASTY
RVVSVLTVLH QDWLNGKEYK CKVSNKALPA PIEKTISKAKG
QPREPQVYT LPPSREEMTK NQVSLTCLVK GFYPSDIAVE
WESNGQPENN YKTTPPVLDS DGSFFLYSKLT VDKSRWQQGN
VFSCSVMHE ALHNHYTQKS LSLSPG
(SEQ ID NO: 52)
VECPPCPAPP VAGPSVFLFP PKPKDTLMIS RTPEVTCVVV
DVSHEDPEVQ FNWYVDGVEV HNAKTKPREE QFNSTFRVVS
VLTVVHQDWL NGKEYKCKVS NKGLPAPIEK TISKTKGQPR
EPQVYTLPPS REEMTKNQVS LTCLVKGFYP SDIAVEWESN
GQPENNYKTT PPMLDSDGSF FLYSKLTVDK SRWQQGNVFS
CSVMHEALHN HYTQKSLSLS PG
(SEQ ID NO: 53)
VECPPCPAPP VAGPSVFLFP PKPKDTLMIS RTPEVTCVVV
DVSHEDPEVQ FNWYVDGVEV HNAKTKPREE QFNSTFRVVS
VLTVVHQDWL NGKEYKCKVS NKGLPSSIEK TISKTKGQPR
EPQVYTLPPS REEMTKNQVS LTCLVKGFYP SDIAVEWESN
GQPENNYKTT PPMLDSDGSF FLYSKLTVDK SRWQQGNVFS
CSVMHEALHN HYTQKSLSLS PG
(SEQ ID NO: 54)
VECPPCPAPP VAGPSVFLFP PKPKDTLMIS RTPEVTCVVV
DVSHEDPEVQ FNWYVDGVEV HNAKTKPREE QFASTFRVVS
VLTVVHQDWL NGKEYKCKVS NKGLPAPIEK TISKTKGQPR
EPQVYTLPPS REEMTKNQVS LTCLVKGFYP SDIAVEWESN
GQPENNYKTT PPMLDSDGSF FLYSKLTVDK SRWQQGNVFS
CSVMHEALHN HYTQKSLSLS PG
(SEQ ID NO: 55)
ESKYGPPCPP CPAPEFLGGP SVFLFPPKPK DTLMISRTPE
VTCVVVDVSQ EDPEVQFNWY VDGVEVHNAK TKPREEQFNS
TYRVVSVLTV LHQDWLNGKE YKCKVSNKGL PSSIEKTISK
AKGQPREPQV YTLPPSQEEM TKNQVSLTCL VKGFYPSDIA
VEWESNGQPE NNYKTTPPVL DSDGSFFLYS RLTVDKSRWQ
EGNVFSCSVM HEALHNHYTQ KSLSLSLG
(SEQ ID NO: 56)
ESKYGPPCPP CPAPEFEGGP SVFLFPPKPK DTLMISRTPE
VTCVVVDVSQ EDPEVQFNWY VDGVEVHNAK TKPREEQFNS
TYRVVSVLTV LHQDWLNGKE YKCKVSNKGL PSSIEKTISK
AKGQPREPQV YTLPPSQEEM TKNQVSLTCL VKGFYPSDIA
VEWESNGQPE NNYKTTPPVL DSDGSFFLYS RLTVDKSRWQ
EGNVFSCSVM HEALHNHYTQ KSLSLSLG
(SEQ ID NO: 92)
DKTHTCPPCP APELLGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPGK
(SEQ ID NO: 93)
DKTHTCPPCP APELLGGPSV FLFPPKPKDT LMISRTPEVT
CVVVAVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPGK
(SEQ ID NO: 94)
DKTHTCPPCP APEAAGAPSV FLFPPKPKDT LMISRTPEVT
CVVVDVSHED PEVKFNWYVD GVEVHNAKTK PREEQYASTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPGK
(SEQ ID NO: 95)
DKTHTCPPCP APEAAGAPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPGK
(SEQ ID NO: 96)
DKTHTCPPCP APELLGGPSV FLFPPKPKDT LMISRTPEVT
CVVVDVSHEDP EVKFNWYVD GVEVHNAKTK PREEQYASTY
RVVSVLTVLH QDWLNGKEYK CKVSNKALPA PIEKTISKAKG
QPREPQVYT LPPSREEMTK NQVSLTCLVK GFYPSDIAVE
WESNGQPENN YKTTPPVLDS DGSFFLYSKLT VDKSRWQQGN
VFSCSVMHE ALHNHYTQKS LSLSPGK
(SEQ ID NO: 97)
VECPPCPAPP VAGPSVFLFP PKPKDTLMIS RTPEVTCVVV
DVSHEDPEVQ FNWYVDGVEV HNAKTKPREE QFNSTFRVVS
VLTVVHQDWL NGKEYKCKVS NKGLPAPIEK TISKTKGQPR
EPQVYTLPPS REEMTKNQVS LTCLVKGFYP SDIAVEWESN
GQPENNYKTT PPMLDSDGSF FLYSKLTVDK SRWQQGNVFS
CSVMHEALHN HYTQKSLSLS PGK
(SEQ ID NO: 98)
VECPPCPAPP VAGPSVFLFP PKPKDTLMIS RTPEVTCVVV
DVSHEDPEVQ FNWYVDGVEV HNAKTKPREE QFNSTFRVVS
VLTVVHQDWL NGKEYKCKVS NKGLPSSIEK TISKTKGQPR
EPQVYTLPPS REEMTKNQVS LTCLVKGFYP SDIAVEWESN
GQPENNYKTT PPMLDSDGSF FLYSKLTVDK SRWQQGNVFS
CSVMHEALHN HYTQKSLSLS PGK
(SEQ ID NO: 99)
VECPPCPAPP VAGPSVFLFP PKPKDTLMIS RTPEVTCVVV
DVSHEDPEVQ FNWYVDGVEV HNAKTKPREE QFASTFRVVS
VLTVVHQDWL NGKEYKCKVS NKGLPAPIEK TISKTKGQPR
EPQVYTLPPS REEMTKNQVS LTCLVKGFYP SDIAVEWESN
GQPENNYKTT PPMLDSDGSF FLYSKLTVDK SRWQQGNVFS
CSVMHEALHN HYTQKSLSLS PGK
(SEQ ID NO: 100)
ESKYGPPCPP CPAPEFLGGP SVFLFPPKPK DTLMISRTPE
VTCVVVDVSQ EDPEVQFNWY VDGVEVHNAK TKPREEQFNS
TYRVVSVLTV LHQDWLNGKE YKCKVSNKGL PSSIEKTISK
AKGQPREPQV YTLPPSQEEM TKNQVSLTCL VKGFYPSDIA
VEWESNGQPE NNYKTTPPVL DSDGSFFLYS RLTVDKSRWQ
EGNVFSCSVM HEALHNHYTQ KSLSLSLGK
(SEQ ID NO: 101)
ESKYGPPCPP CPAPEFEGGP SVFLFPPKPK DTLMISRTPE
VTCVVVDVSQ EDPEVQFNWY VDGVEVHNAK TKPREEQFNS
TYRVVSVLTV LHQDWLNGKE YKCKVSNKGL PSSIEKTISK
AKGQPREPQV YTLPPSQEEM TKNQVSLTCL VKGFYPSDIA
VEWESNGQPE NNYKTTPPVL DSDGSFFLYS RLTVDKSRWQ
EGNVFSCSVM HEALHNHYTQ KSLSLSLGK

In some embodiments, the human Fc variant comprises an amino acid sequence that is at least about any one of 80%, 85%, 90%, 91%, 92%, 93%, 94%, 95%, 96%, 97%, 98%, or 99% identical to any one of SEQ ID NOs: 47-56.

In some embodiments, the human Fc variant binds to an Fcγ receptor with a K_{D} greater than about 5×10⁻⁶ M.

In some embodiments, the decoy polypeptide comprises a human Fc variant that does not cause acute anemia in rodents and non-human primates, e.g., following administration of the decoy polypeptide to a rodent or a non-human primate. In some embodiments, the decoy polypeptide comprises a human Fc variant that does not cause acute anemia in humans, e.g., following administration of the decoy polypeptide to the human. In some embodiments, administration of the decoy polypeptide in vivo results in hemoglobin reduction by less than 50% during the first week after administration. In some embodiments, administration of the polypeptide in humans results in hemoglobin reduction by less than 50% during the first week after administration.

### Exemplary Decoy Polypeptides

In some embodiments, a decoy polypeptide comprises an amino acid sequence set forth in any one of SEQ ID NOs: 57-77. The sequences of SEQ ID NOs: 57-77 are provided below and in Table 2 in Example 1.

(SEQ ID NO: 57)
EEELQIIQPE KLLLVTVGKT ATLHCTITSL FPVGPVLWFR
GVGPGRVLIY NQRQGPFPRV TTVSDTTKRN NMDFSIRISS
ITPADVGTYY CIKFRKGSPE NVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 58)
EEELQIIQPE KLLLVTVGKT ATLHCTITSL FPVGPIQWFR
GVGPGRVLIY NQRDGPFPRV TTVSDGTKRN NMDFSIRISS
ITPADVGTYY CVKFRKGTPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 59)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 60)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 61)
EEELQIIQPE KLLLVTVGKT ATLHCTITSL FPVGPIQWFR
GAGPGRVLIY NQRDGPFPRV TTVSDGTKRN NMDFSIRISS
ITPADVGTYY CIKFRKGTPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 62)
EEELQIIQPE KLLLVTVGKT ATLHCTITSH FPVGPIQWFR
GVGPGRVLIY NQKDGHFPRV TTVSDGTKRN NMDFSIRISS
ITPADVGTYY CVKFRKGSPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 63)
EEELQIIQPE KLLLVTVGKT ATLHCTITSL FPVGPVLWFR
GVGPGRVLIY NQRQGPFPRV TTVSDTTKRN NMDFSIRISS
ITPADVGTYY CVKFRKGTPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 64)
EEELQIIQPE KLLLVTVGKT ATLHCTITSL FPVGPIQWFR
GVGPGRELIY NAREGRFPRV TTVSDLTKRN NMDFSIRISS
ITPADVGTYY CVKFRKGSPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 65)
EEELQIIQPE KLLLVTVGKT ATLHCTITSL FPVGPIQWFR
GVGPGRVLIY NQREGPFPRV TTVSDGTKRN NMDFSIRISS
ITPADVGTYY CVKFRKGSPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 66)
EEELQIIQPE KLLLVTVGKT ATLHCTITSL LPVGPIQWFR
GVGPGRELIY NQRDGPFPRV TTVSDGTKRN NMDFSIRISS
ITPADVGTYY CVKFRKGTPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 67)
EEELQIIQPE KLLLVTVGKT ATLHCTLTSL LPVGPILWFR
GVGPGRVLIY NQRDGPFPRV TTVSDGTKRN NMDFSIRISS
ITPADVGTYY CVKFRKGNPE DVEFKSGPGT EMALGAKPS
DKTHTCPPCP APEAAGAPSV FLFPPKPKDT LMISRTPEVT
CVVVDVSHED PEVKFNWYVD GVEVHNAKTK PREEQYASTY
RVVSVLTVLH QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPG
(SEQ ID NO: 68)
EEELQLIQPE KLLLVTVGKT ATLHCTITSL FPPGPIQWER
GVGPGRVLIY NQKDGPFPRV TTVSDGTKRN NMDFSIRISS
ITPADVGTYY CVKFRKGIPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 69)
EEELQIIQPE KLLLVTVGKT ATLHCTITSL FPIGPILWFR
GVGPGRVLIY NQKDGPFPRV TTVSDGTKRN NMDFSIRISS
ITPADVGTYY CVKFRKGSPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 70)
EEELQMIQPE KLLLVTVGKT ATLHCTITSL FPVGPIQWFR
GAGPGRVLIY NQRDGPFPRV TTVSDGTKRN NMDFSIRISS
ITPADVGTYY CIKFRKGIPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 71)
EEELQMIQPE KLLLVTVGKT ATLHCTVTSL LPVGPVLWFR
GVGPGRELIY NQKEGHFPRV TTVSDLTKRN NMDFSIRISS
ITPADVGTYY CVKFRKGSPE NVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 72)
EDELQIIQPE KSVSVAAGES ATLRCAITSL FPVGPIQWFR
GAGAGRVLIY NQRQGPFPRV TTVSETTKRN NLDFSISISN
ITPADAGTYY CIKFRKGSPD DVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 73)
EEELQIIQPD KSISVAAGES ATLHCTITSL FPVGPIQWFR
GAGPGRVLIY NQRQGPFPRV TTVSDTTKRN NMDFSIRISN
ITPADAGTYY CIKFRKGSPD DVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 74)
EEELQMIQPE KLLLVTVGKT ATLHCTVTSL LPVGPVLWFR
GVGPGRELIY NQKEGHFPRV TTVSDLTKRN NMDFSIRISS
ITPADVGTYY CVKFRKGSPE NVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 75)
EDELQVIQPE KSVSVAAGES ATLRCAMTSL IPVGPIMWFR
GAGAGRELIY NQKEGHFPRV TTVSELTKRN NLDFSISISN
ITPADAGTYY CVKFRKGSPD DVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 76)
EEELQVIQPD KSISVAAGES ATLHCTVTSL IPVGPIQWFR
GAGPGRELIY NQKEGHFPRV TTVSDLTKRN NMDFSIRISN
ITPADAGTYY CVKFRKGSPD HVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 77)
EEELQIIQPD KSVLVAAGET ATLRCTITSL FPVGPIQWFR
GAGPGRELIY NQREGPFPRV TTVSDTTKRN NMDFSIRIGA
ITPADAGTYY CVKFRKGSPD DVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 82)
EEELQIIQPD KSVLVAAGET ATLRCTITSL FPVGPIQWFR
GAGPGRVLIY NQRDGPFPRV TTVSDGTKRN NMDFSIRISS
ITPADVGTYY CVKFRKGTPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 83)
EEELQIIQPD KSVLVAAGET ATLRCTITSL FPVGPIQWFR
GAGPGRVLIY NQRQGPFPRV TTVSDTTKRN NMDFSIRIGN
ITPADAGTYY CIKFRKGSPD DVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 84)
EEELQIIQPD KSVLVAAGET ATLRCTITSL FPVGPIQWFR
GAGPGRVLIY NQRDGPFPRV TTVSDGTKRN NMDFSIRISS
ITPADVGTYY CIKFRKGIPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 85)
EEELQIIQPD KSVLVAAGET ATLRCTITSL FPVGPIQWFR
GAGPGRVLIY NQKDGPFPRV TTVSDGTKRN NMDFSIRISS
ITPADVGTYY CIKFRKGTPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 86)
EEELQIIQPE KLLLVTVGKT ATLRCTITSL FPVGPIQWFR
GAGPGRVLIY NQRDGPFPRV TTVSDGTKRN NMDFSIRISS
ITPADVGTYY CIKFRKGIPE DVEFKSGPGT EMALGAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 90)
EDELQIIQPE KSVSVAAGES ATLRCAITSL FPVGPIQWFR
GAGAGRVLIY NQRQGPFPRV TTVSETTKRN NLDFSISISA
ITPADAGTYY CIKFRKGSPD DVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG
(SEQ ID NO: 91)
EEELQIIQPD KSISVAAGES ATLHCTITSL FPVGPIQWFR
GAGPGRVLIY NQRQGPFPRV TTVSDTTKRN NMDFSIRISA
ITPADAGTYY CIKFRKGSPD DVEFKSGAGT ELSVRAKPSD
KTHTCPPCPA PEAAGAPSVF LFPPKPKDTL MISRTPEVTC
VVVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPG

In some embodiments, a decoy polypeptide that comprises an amino acid sequence that is at least about any one of 80%, 85%, 90%, 91%, 92%, 93%, 94%, 95%, 96%, 97%, 98%, or 99% identical to the amino acid sequence of any one of SEQ ID NOs: 57-77, 82-86, and 90-91.

In some embodiments, the decoy polypeptide comprises a soluble SIRPγ variant that has a K_{D} of about 1×10⁻⁷ M or less (e.g., any one of about 1×10⁻⁸ M or less, 1×10⁻⁹ M or less, 1×10⁻¹⁰ M or less, 1×10⁻¹¹ M or less, 1×10⁻¹² M or less, 1×10⁻¹³ M or less, 1×10⁻¹⁴ M or less, 1×10⁻¹⁵ M or less, or 1×10⁻¹⁶ M or less) affinity for CD47 (e.g., human CD47). In some embodiments, the decoy polypeptide comprises a soluble SIRPγ variant that has an affinity for CD47 in a range of from 1 fM to 1 μM (e.g., from 1 fM to 800 nM, from 10 fM to 500 nM, from 100 fM to 100 nM, from 500 fM to 50 nM, from 800 fM to 50 nM, from 1 pM to 50 nM, from 10 pM to 50 nM, from 50 pM to 50 nM, from 100 pM to 50 nM, from 500 fM to 100 nM, from 800 fM to 100 nM, from 1 pM to 100 nM, from 10 pM to 100 nM, from 50 pM to 100 nM, or from 100 pM to 100 nM). In some embodiments, the decoy polypeptide comprises a soluble SIRPγ variant, that binds to CD47 with an affinity of 1 μM or greater (e.g., 800 nM or greater, 500 nM or greater, 200 nM or greater, 100 nM or greater, 50 nM or greater, 10 nM or greater, 1 nM or greater, 900 pM or greater, 750 pM or greater, 500 pM or greater, 200 pM or greater, 100 pM or greater, 10 pM or greater, 1 pM or greater, etc., where the affinity increases with decreasing values). In some embodiments, the decoy polypeptide that comprises a soluble SIRPγ variant has an affinity for CD47 that is at least about 2-fold greater or more (e.g., at least about any one of 5-, 10-, 100-, 200-, 300-, 400-, 500-, 600-, 700-, 800-, 900-, 1000-, 2000-, 3000-, 4000-, 5000-, 6000-, 7000-, 8000-, 9000-10⁴-, 10⁵-, 10⁶-, 10⁷-, or 10⁸-fold greater or more, etc., including any range in between these values) than the affinity of a wild type SIRPγ protein for CD47. In some embodiments, the decoy polypeptide comprises a soluble SIRPγ variant that has a dissociation half-life for CD47 that is 2-fold greater or (e.g., about any one of 5-fold greater, 10-fold greater, 100-fold greater, 500-fold greater, 1000-fold greater, 5000-fold greater, 10⁴-fold greater, 10⁵-fold greater, 10⁶-fold greater, 10⁷-fold greater, 10⁸-fold greater or more, etc., including any range in between these values) greater than the dissociation half-life for CD47 of a wild type SIRPγ. For example, in some cases, a wild type SIRPγ polypeptide has a dissociation half-life for CD47 of less than 1 second, while a decoy polypeptide described herein comprises a soluble SIRPγ variant that has a dissociation half-life of 5 seconds or more (e.g., 30 seconds or more, 1 minute or more, 5 minutes or more, 10 minutes or more, 20 minutes or more, 30 minutes or more, 40 minutes or more, etc., including any range in between these values). For example, in some embodiments, the amino acid substitution(s)/deletion(s)/insertion(s) in a soluble SIRPγ variant increase the affinity of the decoy polypeptide for binding to CD47 (e.g., as compared to a wild type SIRPγ) by decreasing the off-rate by at least about any one of 10-fold, 20-fold, 50-fold 100-fold 500-fold, 750-fold, 1,000-fold, 2,000-fold, 3,000-fold, 4,000-fold, 5,000-fold, 6,000-fold, 7,000-fold, 8,000-fold, 9,000-fold, 10,000-fold, or more, including any range in between.

In some embodiments, the decoy polypeptide comprises a soluble SIRPβ1 variant that has a K_{D} of about 1×10⁻⁷ M or less (e.g., any one of about 1×10⁻⁸ M or less, 1×10⁻⁹ M or less, 1×10⁻¹⁰ M or less, 1×10⁻¹¹ M or less, 1×10⁻¹² M or less, 1×10⁻¹³ M or less, 1×10⁻¹⁴ M or less, 1×10⁻¹⁵ M or less, or 1×10⁻¹⁶ M or less, including any range in between these values) for CD47 (e.g., human CD47, a CD47 of a non-human primate, such as a cynomolgus monkey, or a mouse CD47). In some embodiments, the decoy polypeptide comprises a soluble SIRPβ1 variant that has a K_{D} of about 0.2-0.3 nM of less for CD47 (e.g., human CD47, a CD47 of a non-human primate, such as a cynomolgus monkey, or a mouse CD47). In some embodiments, the decoy polypeptide comprises a soluble SIRPβ1 variant that has an affinity for CD47 in a range of from 1 fM to 1 μM (e.g., from 1 fM to 800 nM, from 10 fM to 500 nM, from 100 fM to 100 nM, from 500 fM to 50 nM, from 800 fM to 50 nM, from 1 pM to 50 nM, from 10 pM to 50 nM, from 50 pM to 50 nM, from 100 pM to 50 nM, from 500 fM to 100 nM, from 800 fM to 100 nM, from 1 pM to 100 nM, from 10 pM to 100 nM, from 50 pM to 100 nM, or from 100 pM to 100 nM). In some embodiments, the decoy polypeptide comprises a soluble SIRPβ1 variant, that binds to CD47 with an affinity of 1 μM or greater (e.g., 800 nM or greater, 500 nM or greater, 200 nM or greater, 100 nM or greater, 50 nM or greater, 10 nM or greater, 1 nM or greater, 900 pM or greater, 750 pM or greater, 500 pM or greater, 200 pM or greater, 100 pM or greater, 10 pM or greater, 1 pM or greater, etc., where the affinity increases with decreasing values). In some embodiments, the decoy polypeptide that comprises a soluble SIRPβ1 variant has an affinity for CD47 that is at least about 2-fold greater or more (e.g., at least about any one of 5-, 10-, 100-, 200-, 300-, 400-, 500-, 600-, 700-, 800-, 900-, 1000-, 2000-, 3000-, 4000-, 5000-, 6000-, 7000-, 8000-, 9000-10⁴-, 10⁵-, 10⁶-, 10⁷-, or 10⁸-fold greater or more, etc., including any range in between these values) than the affinity of a wild type SIRPβ1 protein for CD47. In some embodiments, the decoy polypeptide comprises a soluble SIRPβ1 variant that has a dissociation half-life for CD47 that is 2-fold greater or (e.g., about any one of 5-fold greater, 10-fold greater, 100-fold greater, 500-fold greater, 1000-fold greater, 5000-fold greater, 10⁴-fold greater, 10⁵-fold greater, 10⁶-fold greater, 10⁷-fold greater, 10⁸-fold greater or more, etc., including any range in between these values) greater than the dissociation half-life for CD47 of a wild type SIRPβ1. For example, in some cases, the wild type SIRPβ1 polypeptide does not bind CD47, while a decoy polypeptide described herein comprises a soluble SIRPβ1 variant that has a dissociation half-life of 5 seconds or more (e.g., 30 seconds or more, 1 minute or more, 5 minutes or more, 10 minutes or more, 20 minutes or more, 30 minutes or more, 40 minutes or more, etc., including any range in between these values). For example, in some embodiments, the amino acid substitution(s)/deletion(s)/insertion(s) in a soluble SIRPβ1 variant increase the affinity of the decoy polypeptide for binding to CD47 (e.g., as compared to a wild type SIRPβ1) by decreasing the off-rate by at least about any one of 10-fold, 20-fold, 50-fold 100-fold 500-fold, 750-fold, 1,000-fold, 2,000-fold, 3,000-fold, 4,000-fold, 5,000-fold, 6,000-fold, 7,000-fold, 8,000-fold, 9,000-fold, 10,000-fold, including any range in between.

In some embodiments, the decoy polypeptide comprises a soluble SIRPβ2 variant that has a K_{D} of about 1×10⁻⁷ M or less (e.g., any one of about 1×10⁻⁸ M or less, 1×10⁻⁹ M or less, 1×10⁻¹⁰ M or less, 1×10⁻¹¹ M or less, 1×10⁻¹² M or less, 1×10⁻¹³ M or less, 1×10⁻¹⁴ M or less, 1×10⁻¹⁵ M or less, or 1×10⁻¹⁶ M or less, including any range in between these values) for CD47 (e.g., human CD47, a CD47 of a non-human primate, such as a cynomolgus monkey, or a mouse CD47). In some embodiments, the decoy polypeptide comprises a soluble SIRPβ2 variant that has a K_{D} of about 0.2-0.3 nM of less for CD47 (e.g., human CD47, a CD47 of a non-human primate, such as a cynomolgus monkey, or a mouse CD47). In some embodiments, the decoy polypeptide comprises a soluble SIRPβ2 variant that has an affinity for CD47 in a range of from 1 fM to 1 μM (e.g., from 1 fM to 800 nM, from 10 fM to 500 nM, from 100 fM to 100 nM, from 500 fM to 50 nM, from 800 fM to 50 nM, from 1 pM to 50 nM, from 10 pM to 50 nM, from 50 pM to 50 nM, from 100 pM to 50 nM, from 500 fM to 100 nM, from 800 fM to 100 nM, from 1 pM to 100 nM, from 10 pM to 100 nM, from 50 pM to 100 nM, or from 100 pM to 100 nM). In some embodiments, the decoy polypeptide comprises a soluble SIRPβ2 variant, that binds to CD47 with an affinity of 1 μM or greater (e.g., 800 nM or greater, 500 nM or greater, 200 nM or greater, 100 nM or greater, 50 nM or greater, 10 nM or greater, 1 nM or greater, 900 pM or greater, 750 pM or greater, 500 pM or greater, 200 pM or greater, 100 pM or greater, 10 pM or greater, 1 pM or greater, etc., where the affinity increases with decreasing values). In some embodiments, the decoy polypeptide that comprises a soluble SIRPβ2 variant has an affinity for CD47 that is at least about 2-fold greater or more (e.g., at least about any one of 5-, 10-, 100-, 200-, 300-, 400-, 500-, 600-, 700-, 800-, 900-, 1000-, 2000-, 3000-, 4000-, 5000-, 6000-, 7000-, 8000-, 9000-10⁴-, 10⁵-, 10⁶-, 10⁷-, or 10⁸-fold greater or more, etc., including any range in between these values) than the affinity of a wild type SIRPβ2 protein for CD47. In some embodiments, the decoy polypeptide comprises a soluble SIRPβ2 variant that has a dissociation half-life for CD47 that is 2-fold greater or (e.g., about any one of 5-fold greater, 10-fold greater, 100-fold greater, 500-fold greater, 1000-fold greater, 5000-fold greater, 10⁴-fold greater, 10⁵-fold greater, 10⁶-fold greater, 10⁷-fold greater, 10⁸-fold greater or more, etc., including any range in between these values) greater than the dissociation half-life for CD47 of a wild type SIRPβ2. For example, in some cases, a wild type SIRPβ2 polypeptide does note bind CD47, while a decoy polypeptide described herein comprises a soluble SIRPβ2 variant that has a dissociation half-life of 5 seconds or more (e.g., 30 seconds or more, 1 minute or more, 5 minutes or more, 10 minutes or more, 20 minutes or more, 30 minutes or more, 40 minutes or more, etc., including any range in between these values). For example, in some embodiments, the amino acid substitution(s)/deletion(s)/insertion(s) in a soluble SIRPβ2 variant increase the affinity of the decoy polypeptide for binding to CD47 (e.g., as compared to a wild type SIRPβ2) by decreasing the off-rate by at least about any one of 10-fold, 20-fold, 50-fold 100-fold 500-fold, 750-fold, 1,000-fold, 2,000-fold, 3,000-fold, 4,000-fold, 5,000-fold, 6,000-fold, 7,000-fold, 8,000-fold, 9,000-fold, 10,000-fold, or more, including any range in between.

### Chimeric Molecules Comprising a Decoy Polypeptide

In some embodiments, a decoy polypeptide is modified in a way to form a chimeric molecule comprising the decoy polypeptide fused (e.g., recombinantly fused) to another, heterologous polypeptide or amino acid sequence. In certain embodiments, a chimeric molecule comprises a fusion of a decoy polypeptide with a second moiety (such as a protein transduction domain) which targets the chimeric molecule for delivery to various tissues, or, e.g., across brain blood barrier, using, for example, the protein transduction domain of human immunodeficiency virus TAT protein (Schwarze et al., 1999, Science 285: 1569-72). In certain embodiments, a chimeric molecule comprises a fusion of a decoy polypeptide with a signal sequence or leader sequence so that the decoy polypeptide may be secreted by the cell in which it is expressed.

In certain embodiments, a decoy polypeptide provided herein can be used as bi- or multi-specific (for different target ligands or different epitopes on the same target ligand) in multimer form. For example, a bispecific decoy polypeptide comprises one subunit with specificity for a first target protein or epitope and a second subunit with specificity for a second target protein or epitope. Decoy polypeptides can be joined in a variety of conformations that can increase the valency and thus the avidity of binding to a target ligand.

In certain embodiments a chimeric molecule provided herein comprises two or more (such as three, four, five, six, seven, eight, nine, ten, or more than ten) decoy polypeptides. In certain embodiments, a nucleic acid can be engineered to encode two or more copies of a single decoy polypeptide, which copies are transcribed and translated in tandem to produce a covalently linked multimer of identical subunits. In certain embodiments, the nucleic acid can be engineered to encode two or more different non-naturally occurring CKPs, which copies are transcribed and translated in tandem to produce a covalently linked multimer of different subunits.

In another embodiment, such a chimeric molecule comprises a fusion of a decoy polypeptide with a tag polypeptide which provides an epitope to which an anti-tag antibody can selectively bind. The epitope tag is generally placed at the amino- or carboxyl-terminus of the decoy polypeptide. The presence of such epitope-tagged forms of the decoy polypeptide can be detected using an antibody against the tag polypeptide. Also, provision of the epitope tag enables the decoy polypeptide to be readily purified by affinity purification using an anti-tag antibody or another type of affinity matrix that binds to the epitope tag. Various tag polypeptides and their respective antibodies are known in the art. Examples include poly-histidine (poly-His) (e.g., HHHHHHHH (SEQ ID NO: 40)) or poly-histidine-glycine (poly-His-Gly) tags; a biotin acceptor peptide tag (GLNDIFEAQKIEWHE (SEQ ID NO: 41)); the flu HA tag polypeptide and its antibody 12CA5 (Field et al. (1988) Mol. Cell. Biol. 8, 2159-2165); the c-myc tag and the 8F9, 3C7, 6E10, G4, B7 and 9E10 antibodies thereto (Evan et al. (1985) Mol. Cell. Biol. 5, 3610-3616]; and the Herpes Simplex virus glycoprotein D (gD) tag and its antibody (Paborsky et al. (1990) Protein Eng., 3, 547-553). Other tag polypeptides include the Flag-peptide (Hopp et al. (1988) BioTechnology, 6,1204-1210); the KT3 epitope peptide (Martin et al. (1992) Science, 255, 192-194]; an α-tubulin epitope peptide (Skinner et al. (1991) J. Biol. Chem. 266, 15163-15166); and the T7 gene 10 protein peptide tag (Lutz-Freyermuth et al. (1990) Proc. Natl. Acad. Sci. USA 87, 6393-6397].

In certain embodiments, a decoy polypeptide described herein is fused with a molecule that increases or extends in vivo or serum half-life. In certain embodiments, a decoy polypeptide is fused with albumin, such as human serum albumin (HSA), polyethylene glycol (PEG), polysaccharides, complement, hemoglobin, a binding peptide, lipoproteins or other factors to increase its half-life in the bloodstream and/or its tissue penetration.

In certain embodiments, a decoy polypeptide provided herein is altered by being subjected to random mutagenesis by error-prone PCR, random nucleotide insertion or other methods prior to recombination. One or more portions of a polynucleotide encoding a scaffold that binds to a specific target may be recombined with one or more components, motifs, sections, parts, domains, fragments, etc. of one or more heterologous molecules.

Any of these fusions can generated by standard techniques, for example, by expression of the fusion protein from a recombinant fusion gene constructed using publicly available gene sequences, or by chemical peptide synthesis.

Exemplary heterologous polypeptides that may be fused to a decoy polypeptide described herein include, without limitation, e.g., Glutathione S-transferase (GST), beta-galactosidase, a yeast two-hybrid GAL fusion, a poly-His tag. In some embodiments, the heterologous polypeptide linked to the decoy polypeptide may alter (e.g., enhance or dampen) the ability of the SIRPγ variant, the SIRPβ1 variant, or SIRPβ2 variant to bind CD47. In some embodiments, the heterologous polypeptide fused to the decoy polypeptide may alter the activity that the SIRPγ variant, the SIRPβ1 variant, or SIRPβ2 variant of the decoy polypeptide imparts on myeloid cell activity including phagocytosis and ADCC. In some embodiments, the decoy polypeptide is linked to a green fluorescent protein or a red fluorescent protein. In some embodiments, the decoy polypeptide is linked to a wild type subunit of PD-1 (PDCD1), PD-L1 (CD274), PD-L2 (PDCDILG2), CTLA4, TIM3 (HAVCR2), CEACAMI, LAG3, BTLA, TNFRSF14, TIGIT, PVR, LIGHT, IL2, IL12A, IL15, IL10, LILRB1, LILRB2, LILRB3, LILRB4, LILRB5, LILRA1, LILRA2, LILRA3, LILRA4, LILRA5, LILRA6, CD40, CD40L, OX40, OX40L, CD137 (4-1BB, TNFRSF9), TNFSF9 (4-1BBL), B7-H4 (VCTN1), SIRPA, CD47, CD33, CD44, C5, C3, or other immune regulatory proteins. In some embodiments, the decoy polypeptide further comprises a variant of PD-1 (PDCD1), PD-L1 (CD274), PD-L2 (PDCDILG2), CTLA4, TIM3 (HAVCR2), CEACAM1, LAG3, BTLA, TNFRSF14, TIGIT, PVR, LIGHT, IL2, IL12A, IL15, IL10, LILRB1, LILRB2, LILRB3, LILRB4, LILRB5, LILRA1, LILRA2, LILRA3, LILRA4, LILRA5, LILRA6, CD40, CD40L, OX40, OX40L, CD137 (4-1BB, TNFRSF9), TNFSF9 (4-1BBL), B7-H4 (VCTN1), SIRPA, CD47, CD33, CD44, C5, C3, or other immune regulatory proteins, engineered for high affinity binding to their respective ligands. In some embodiments, the decoy polypeptide further comprises a variant of PD-1 (PDCD1), PD-L1 (CD274), PD-L2 (PDCDILG2), CTLA4, TIM3 (HAVCR2), CEACAM1, LAG3, BTLA, TNFRSF14, TIGIT, PVR, LIGHT, IL2, IL12A, IL15, IL10, LILRB1, LILRB2, LILRB3, LILRB4, LILRB5, LILRA1, LILRA2, LILRA3, LILRA4, LILRA5, LILRA6, CD40, CD40L, OX40, OX40L, CD137 (4-1BB, TNFRSF9), TNFSF9 (4-1BBL), B7-H4 (VCTN1), SIRPA, CD47, CD33, CD44, C5, C3, or other immune regulatory proteins, engineered for reduced affinity binding to their respective ligands. In some embodiments, the decoy polypeptide further comprises a variant of PD-1 (PDCD1), PD-L1 (CD274), PD-L2 (PDCDILG2), CTLA4, TIM3 (HAVCR2), CEACAMI, LAG3, BTLA, TNFRSF14, TIGIT, PVR, LIGHT, IL2, IL12A, IL15, IL10, LILRB1, LILRB2, LILRB3, LILRB4, LILRB5, LILRA1, LILRA2, LILRA3, LILRA4, LILRA5, LILRA6, CD40, CD40L, OX40, OX40L, CD137 (4-1BB, TNFRSF9), TNFSF9 (4-1BBL), B7-H4 (VCTN1), SIRPA, CD47, CD33, CD44, C5, C3 or other immune regulatory proteins, engineered for altered binding affinity to additional ligands besides their natural ligands.

In some embodiments, the decoy polypeptide is linked to a monoclonal antibody, e.g., an anti-CD20 antibody, an anti-EGFR antibody, an anti-Her2/Neu (ERBB2) antibody, an anti-EPCAM antibody, an anti-GL2 antibody, anti-GD2, anti-GD3, anti-CD2, anti-CD3, anti-CD4, anti-CD8, anti-CD I 9, anti-CD22, anti-CD30, anti-CD33, anti-CD45, anti-CD47, anti-CD52, anti-CD56, anti-CD70, anti-CD117, an anti-SIRPA antibody, an anti-CD47 antibody, an anti-LILRB1 antibody, an anti-PD-1 antibody, an anti-PD-L1 antibody, an anti-PD-L2 antibody, or any antibody designed to bind to a tumor cell, a virally- or bacterially-infected cell, immune cell, or healthy normal cell, or to a cytokine, chemokine, or hormone of any kind.

In some embodiments, the decoy polypeptide further comprises a polypeptide sequence comprising an immune checkpoint inhibitor, a co-stimulatory molecule, or a cytokine or an attenuated cytokine. In some embodiments, the decoy polypeptide and the polypeptide sequence comprising an immune checkpoint inhibitor, a co-stimulatory molecule, or a cytokine or an attenuated cytokine are linked by a Gly-Ser linker of varying length and composition. In some embodiments, the linker sequence comprises the sequence GGGGSGGGGS (SEQ ID NO: 29). The order of the polypeptide sequences at the N- or C-terminus may also be varied. The amino acid sequences of exemplary decoy polypeptides comprising immune checkpoint inhibitors (or portions thereof), co-stimulatory molecules (or portions thereof), or cytokines or attenuated cytokines (or portions thereof) are provided below:

### Decoy Polypeptides Comprising Immune Checkpoint Inhibitors

### a. PD-1/PD-L1 Antagonist

### Example: HAC-GV3 (High-Affinity PD-1 Decoy Fused to GV3)

(SEQ ID NO: 30)
DSPDRPWNPP TFSPALLVVT EGDNATFTCS FSNTSESFHV
VWHRESPSGQ TDTLAFPEDR SQPGQDARFR VTQLPNGRDF
HMSVVRARRN DSGTYVCGVI SLAPKIQIKE SLRAELRVTE
RGGGGSGGGG SEEELQIIQP EKLLLVTVGK TATLHCTITS
LFPVGPIQWF RGVGPGRVLI YNQKDGHFPR VTTVSDGTKR
NNMDFSIRIS SITPADVGTY YCVKFRKGSP EDVEFKSGPG
TEMALGAKPS

### Example: HAC+SIRPγ Variant (High-Affinity PD-1 Decoy Fused to the SIRPγ Variant of SEQ ID NO: 5)

(SEQ ID NO: 102)
DSPDRPWNPP TFSPALLVVT EGDNATFTCS FSNTSESFHV
VWHRESPSGQ TDTLAFPEDR SQPGQDARFR VTQLPNGRDF
HMSVVRARRN DSGTYVCGVI SLAPKIQIKE SLRAELRVTE
RGGGGSGGGG SEEELQIIQPE KLLLVTVGKT ATLHCTITSL
FPVGPIQWFR GVGPGRVLIY NQKDGPFPRV TTVSDGTKRN
NMDFSIRISS ITPADVGTYY CVKFRKGSPE DVEFKSGPGT
EMALGAKPS

### b. BTLA/CD160 Antagonist

### Example: GV3-BTLA Decoy

(SEQ ID NO: 31)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSW NIHGKESCDV QLYIKRQSEH SILAGDPFEL
ECPVKYCANR PHVTWCKLNG TTCVKLEDRQ TSWKEEKNIS
FFILHFEPVL PNDNGSYRCS ANFQSNLIES HSTTLYVTDV K

### Example: SIRPγ Variant-BTLA Decoy (Comprising the SIRPγ Variant of SEQ ID NO: 5)

(SEQ ID NO: 103)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSW NIHGKESCDV QLYIKRQSEH SILAGDPFEL
ECPVKYCANR PHVTWCKLNG TTCVKLEDRQ TSWKEEKNIS
FFILHFEPVL PNDNGSYRCS ANFQSNLIES HSTTLYVTDV K

### c. Phosphatidylserine Antagonist

### Example: GV3-MFGE8 Decoy

(SEQ ID NO: 32)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSE LNGCANPLGL KNNSIPDKQI TASSSYKTWG
LHLFSWNPSY ARLDKQGNFN AWVAGSYGND QWLQVDLGSS
KEVTGIITQG ARNFGSVQFV ASYKVAYSND SANWTEYQDP
RTGSSKIFPG NWDNHSHKKN LFETPILARY VRILPVAWHN
RIALRLELLG C

### Example: SIRPγ Variant—MFGE8 Decoy (Comprising the SIRPγ Variant of SEQ ID NO: 5)

(SEQ ID NO: 104)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSE LNGCANPLGL KNNSIPDKQI TASSSYKTWG
LHLFSWNPSY ARLDKQGNFN AWVAGSYGND QWLQVDLGSS
KEVTGIITQG ARNFGSVQFV ASYKVAYSND SANWTEYQDP
RTGSSKIFPG NWDNHSHKKN LFETPILARY VRILPVAWHN
RIALRLELLG C

### Example: GV3-Tim 1 Decoy

(SEQ ID NO: 33)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSV AGSVKVGGEA GPSVTLPCHY SGAVTSMCWN
RGSCSLFTCQ NGIVWTNGTH VTYRKDTRYK LLGDLSRRDV
SLTIENTAVS DSGVYCCRVE HRGWENDMKI TVSLEIVPPK VTT

### Example: SIRPγ Variant-Tim 1 Decoy (Comprising the SIRPγ Variant of SEQ ID NO: 5)

(SEQ ID NO: 105)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSV AGSVKVGGEA GPSVTLPCHY SGAVTSMCWN
RGSCSLFTCQ NGIVWTNGTH VTYRKDTRYK LLGDLSRRDV
SLTIENTAVS DSGVYCCRVE HRGWENDMKI TVSLEIVPPK VTT

### Example: GV3-Tim3 Decoy

(SEQ ID NO: 34)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSS EVEYRAEVGQ NAYLPCFYTP AAPGNLVPVC
WGKGACPVFE CGNVVLRTDE RDVNYWTSRY WLNGDFRKGD
VSLTIENVTL ADSGIYCCRI QIPGIMNDEK FNLKLVIKPA KVTPA

### Example: SIRPγ Variant-Tim 3 Decoy (Comprising the SIRPγ Variant of SEQ ID NO: 5)

(SEQ ID NO: 106)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSS EVEYRAEVGQ NAYLPCFYTP AAPGNLVPVC
WGKGACPVFE CGNVVLRTDE RDVNYWTSRY WLNGDFRKGD
VSLTIENVTL ADSGIYCCRI QIPGIMNDEK FNLKLVIKPA KVTPA

### Example: GV3-Tim4 Decoy

(SEQ ID NO: 35)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGST SETVVTEVLG HRVTLPCLYS SWSHNSNSMC
WGKDQCPYSG CKEALIRTDG MRVTSRKSAK YRLQGTIPRG
DVSLTILNPS ESDSGVYCCR IEVPGWENDV KINVRLNLQR
ASTTTDEKFN LKLVIKPAKV TPA

### Example: SIRPγ Variant-Tim 4 Decoy (Comprising the SIRPγ Variant of SEQ ID NO: 5)

(SEQ ID NO: 107)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGST SETVVTEVLG HRVTLPCLYS SWSHNSNSMC
WGKDQCPYSG CKEALIRTDG MRVTSRKSAK YRLQGTIPRG
DVSLTILNPS ESDSGVYCCR IEVPGWENDV KINVRLNLQR
ASTTTDEKFN LKLVIKPAKV TPA

### Decoy Polypeptides Comprising Co-Stimulatory Molecules

### 2) Fusion to Co-Stimulatory Molecules

### a. CD40 Agonist

### Example: GV3-CD40L

(SEQ ID NO: 36)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSG DQNPQIAAHV ISEASSKTTS VLQWAEKGYY
TMSNNLVTLE NGKQLTVKRQ GLYYIYAQVT FCSNREASSQ
APFIASLCLK SPGRFERILL RAANTHSSAK PCGQQSIHLG
GVFELQPGAS VFVNVTDPSQ VSHGTGFTSF GLLKL

### Example: SIRPγ Variant-CD40L (Comprising the SIRPγ Variant of SEQ ID NO: 5)

(SEQ ID NO: 108)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSG DQNPQIAAHV ISEASSKTTS VLQWAEKGYY
TMSNNLVTLE NGKQLTVKRQ GLYYIYAQVT FCSNREASSQ
APFIASLCLK SPGRFERILL RAANTHSSAK PCGQQSIHLG
GVFELQPGAS VFVNVTDPSQ VSHGTGFTSF GLLKL

### b. 41BB (CD137) Agonist

### Example: GV3-41BBL

(SEQ ID NO: 37)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSD PAGLLDLRQG MFAQLVAQNV LLIDGPLSWY
SDPGLAGVSL TGGLSYKEDT KELVVAKAGV YYVFFQMELR
RVVAGEGSGS VSLALHLMPL RSAAGAAALA LTVDLPPASS
EARNSAFGFQ GRLLHLSAGQ RLGVHLHTEA RARHAWQLTQ
GATVLGLFRV TPEIPA

### Example: SIRPγ Variant-41BBL (Comprising the SIRPγ Variant of SEQ ID NO: 5)

(SEQ ID NO: 109)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSD PAGLLDLRQG MFAQLVAQNV LLIDGPLSWY
SDPGLAGVSL TGGLSYKEDT KELVVAKAGV YYVFFQMELR
RVVAGEGSGS VSLALHLMPL RSAAGAAALA LTVDLPPASS
EARNSAFGFQ GRLLHLSAGQ RLGVHLHTEA RARHAWQLTQ
GATVLGLFRV TPEIPA

### Decoy Polypeptides Comprising Cytokines or Attenuated Cytokines

### 3) Fusion to Cytokines and Attenuated Cytokines

### Example: GV3-IL2

(SEQ ID NO: 38)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSA PTSSSTKKTQ LQLEHLLLDL QMILNGINNY
KNPKLTRMLT FKFYMPKKAT ELKHLQCLEE ELKPLEEVLN
LAQSKNFHLR PRDLISNINV IVLELKGSET TFMCEYADET
ATIVEFLNRW ITFCQSIIST LT

### Example: SIRPγ Variant-IL2 (Comprising the SIRPγ Variant of SEQ ID NO: 5)

(SEQ ID NO: 110)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSA PTSSSTKKTQ LQLEHLLLDL QMILNGINNY
KNPKLTRMLT FKFYMPKKAT ELKHLQCLEE ELKPLEEVLN
LAQSKNFHLR PRDLISNINV IVLELKGSET TFMCEYADET
ATIVEFLNRW ITFCQSIIST LT

### Example: GV3-IL2 (an “Attenuated” Cytokine with Mutations F42A/D20T)

(SEQ ID NO: 39)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPSG
GGGSGGGGSA PTSSSTKKTQ LQLEHLLLTL QMILNGINNY
KNPKLTRMLT AKFYMPKKAT ELKHLQCLEE ELKPLEEVLN
LAQSKNFHLR PRDLISNINV IVLELKGSET TFMCEYADET
ATIVEFLNRW ITFCQSIIST LT

### Example: SIRPγ Variant-IL2 (Comprising the SIRPγ Variant of SEQ ID NO: 5 and an “Attenuated” Cytokine with Mutations F42A/D20T)

(SEQ ID NO: 111)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPSG
GGGSGGGGSA PTSSSTKKTQ LQLEHLLLTL QMILNGINNY
KNPKLTRMLT AKFYMPKKAT ELKHLQCLEE ELKPLEEVLN
LAQSKNFHLR PRDLISNINV IVLELKGSET TFMCEYADET
ATIVEFLNRW ITFCQSIIST LT

### Conjugates Comprising a Decoy Polypeptide

Provided herein are conjugates comprising a decoy polypeptide described herein conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate). In some embodiments, the conjugate comprises a SIRPγ, SIRPβ1, or a SIRPβ2 variant described herein, a decoy polypeptide described herein, or a chimeric molecule that comprises a SIRPγ, SIRPβ1, or a SIRPβ2 variant described herein or a decoy polypeptide described herein.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes. Other toxins include maytansine and maytansinoids, calicheamicin and other cytotoxic agents. A variety of radionuclides are available for the production of radioconjugated decoy polypeptides. Examples include ²¹²Bi, ¹³¹I, ¹³¹In, ⁹⁰Y, and ¹⁸⁶Re.

Conjugates of a decoy polypeptide described herein and, e.g., cytotoxic agent, are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bisdiazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionuclide to a decoy polypeptide. See, WO94/11026.

In another embodiment, the decoy polypeptide can be conjugated to a “receptor” (such as streptavidin) for utilization in ocular “pre-targeting” wherein the non-naturally occurring EETI-II scaffold protein-receptor conjugate is administered to the eye patient, followed by removal of unbound conjugate from the circulation using a clearing agent and then administration of a “ligand” (e.g., avidin) that is conjugated to a cytotoxic agent (e.g., a radionuclide) or a therapeutic agent.

In certain embodiments, the decoy polypeptide provided herein can be used as bi- or multi-specific (for different target ligands or different epitopes on the same target ligand) in multimer form. The attachments may be covalent or non-covalent. For example, a dimeric bispecific decoy polypeptide has one subunit with specificity for a first target protein or epitope and a second subunit with specificity for a second target protein or epitope. Decoy polypeptides can be joined, e.g., via conjugation, in a variety of conformations that can increase the valency and thus the avidity of binding to a target ligand or to bind multiple target ligands.

In certain embodiments, decoy polypeptides provided herein are engineered to provide reactive groups for conjugation. In certain embodiments, the N-terminus and/or C-terminus may also serve to provide reactive groups for conjugation. In certain embodiments, the N-terminus is conjugated to one moiety (such as, but not limited to PEG) while the C-terminus is conjugated to another moiety (such as, but not limited to biotin), or vice versa.

Provided is a decoy polypeptide described herein conjugated to one or more moieties, including but not limited to, peptides, polypeptides, proteins, fusion proteins, nucleic acid molecules, small molecules, mimetic agents, synthetic drugs, inorganic molecules, and organic molecules. Also provided a decoy polypeptide described chemically conjugated (including both covalent and non-covalent conjugations) to a heterologous protein or polypeptide (or fragment thereof, to a polypeptide of at least 10, at least 20, at least 30, at least 40, at least 50, at least 60, at least 70, at least 80, at least 90 or at least 100 amino acids). The fusion does not necessarily need to be direct, but may occur through linker sequences described herein.

In certain embodiments, decoy polypeptide described herein, or analogs or derivatives thereof may be conjugated to a diagnostic or detectable agent. Such decoy polypeptide conjugates can be useful for monitoring or prognosing the development or progression of a disease as part of a clinical testing procedure, such as determining the efficacy of a particular therapy. Such diagnosis and detection can be accomplished by coupling the decoy polypeptide to detectable substances including, but not limited to various enzymes, such as but not limited to horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as but not limited to iodine (¹³¹I, ¹²⁵I, ¹²⁴I, ¹²³I, ¹²¹I) carbon (¹¹C, ¹⁴C), sulfur (³⁵S), tritium (³H), indium (¹¹⁵In, ¹¹³In, ¹¹²In ¹¹¹In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹²Pr, ¹⁰⁵Rh ⁹⁷Ru, ⁶⁸Ge, ⁵⁷Co, ⁶⁴Cu, ⁶⁵Zn, ⁸⁹Zr, ⁸⁵Sr, ³²P, ³³P, ³H, ¹⁵³Gd, ¹⁶⁹Yb, ⁵¹Cr, ⁵⁴Mn, ⁷⁵Se, ¹¹³Sn, ¹¹⁷Tn, ¹³N, ¹⁵O, ⁶²Cu, ⁷⁶Br, and ⁸²Rb; positron emitting metals using various positron emission tomographies, nonradioactive paramagnetic metal ions, and molecules that are radiolabeled or conjugated to specific radioisotopes. In some embodiments, the decoy polypeptide is conjugated to, e.g., Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, BODIPY® FL, Coumarin, Cy®3, Cy®5, Fluorescein (FITC), Oregon Green®, Pacific Blue™, Pacific Green™, Pacific Orange™, Tetramethylrhodamine (TRITC), Texas Red® or other fluorescent label. In some embodiments, the decoy polypeptide is conjugated to a detectable label that comprises a chelating group, such as Cyclen, Cyclam, DO2A, DOTP, DOTMA, TETA, DOTAM, CB-T2A, DOTA or NOTA

Also provided is a decoy polypeptide conjugated to a therapeutic moiety. In certain embodiments, a decoy polypeptide may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

In certain embodiments, a decoy polypeptide is conjugated to therapeutic moieties such as a radioactive metal ion, such as alpha-emitters such as ²¹³Bi or macrocyclic chelators useful for conjugating radiometal ions, including but not limited to, ¹³¹In, ¹³¹Lu, ¹³¹Y ¹³¹Ho, ¹³¹Sm, to polypeptides. In certain embodiments, the macrocyclic chelator is 1, 4, 7, 10-tetraazacyclododecane-N,N′,N″,N′″-tetra-acetic acid (DOTA) which can be attached to the decoy polypeptide via a linker molecule. Such linker molecules are commonly known in the art and described in, e.g., Denardo et al. (1998) Clin Cancer Res. 4, 2483-90; Peterson et al. (1999) Bioconjug. Chem. 10, 553-557; and Zimmerman et al. (1999) Nucl. Med. Biol. 26, 943-50.

Techniques for conjugating therapeutic moieties to antibodies are well known and can be applied to the decoy polypeptides disclosed herein, see, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56. (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radio labeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58. Similar approaches may be adapted for use with the decoy polypeptides provided herein.

The therapeutic moiety or drug conjugated to a decoy polypeptide should be chosen to achieve the desired prophylactic or therapeutic effect(s) for a particular disorder in a subject. A clinician or other medical personnel should consider the following when deciding on which therapeutic moiety or drug to conjugate to a scaffold: the nature of the disease, the severity of the disease, and the condition of the subject.

In certain embodiments, a decoy polypeptide described herein can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Covalent Modifications

Covalent modifications of decoy polypeptide described herein are also contemplated. One type of covalent modification includes reacting targeted amino acid residues of a decoy polypeptide with an organic derivatizing agent that is capable of reacting with selected side chains or the N- or C-terminal residues of the decoy polypeptide. Derivatization with bifunctional agents is useful, for instance, for crosslinking the decoy polypeptide to a water-insoluble support matrix or surface for use in the method for purifying a target ligand, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)-dithio]propioimidate.

Other modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, ubiquitination, deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Covalent modifications may be made anywhere in the SIRPγ variant, the SIRPβ1 variant, or the SIRPβ2 variant, including, for example, the peptide backbone, the amino acid side-chains, and the amino and/or carboxyl termini. Exemplary peptide modifications that may be made to a SIRPγ variant, a SIRPβ1 variant, or a SIRPβ2 variant include, but are not limited to, e.g., glycosylation, lipid attachment, sulfation, gamma-carboxylation of glutamic acid residues, hydroxylation, blockage of the amino or carboxyl group in a polypeptide, or both, by a covalent modification, and ADP-ribosylation.

Another type of covalent modification of a decoy polypeptide comprises linking the decoy polypeptide to one of a variety of nonproteinaceous polymers, e.g., polyethylene glycol (PEG), polypropylene glycol, or polyoxyalkylenes, in the manner set forth in U.S. Pat. No. 4,640,835, 4,496,689, 4,301,144, 4,670,417, 4,791,192 or 4,179,337

The term “polyethylene glycol” or “PEG” means a polyethylene glycol compound or a derivative thereof, with or without coupling agents, coupling or activating moieties (e.g., with thiol, triflate, tresylate, azirdine, oxirane, N-hydroxysuccinimide or a maleimide moiety). The term “PEG” is intended to indicate polyethylene glycol of a molecular weight between 500 and 150,000 Da, including analogues thereof, wherein for instance the terminal OR-group has been replaced by a methoxy group (referred to as mPEG).

In certain embodiments, decoy polypeptides are derivatized with polyethylene glycol (PEG). PEG is a linear, water-soluble polymer of ethylene oxide repeating units with two terminal hydroxyl groups. PEGs are classified by their molecular weights which typically range from about 500 daltons to about 40,000 daltons. In a presently preferred embodiment, the PEGs employed have molecular weights ranging from 5,000 daltons to about 20,000 daltons. PEGs coupled to the decoy polypeptides described herein can be either branched or unbranched (for example, Monfardini, C. et al. 1995 Bioconjugate Chem 6:62-69). PEGs are commercially available from Nektar Inc., Sigma Chemical Co. and other companies. Such PEGs include, but are not limited to, monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, the hydrophilic polymer which is employed, for example, PEG, is capped at one end by an unreactive group such as a methoxy or ethoxy group. Thereafter, the polymer is activated at the other end by reaction with a suitable activating agent, such as cyanuric halides (for example, cyanuric chloride, bromide or fluoride), diimadozle, an anhydride reagent (for example, a dihalosuccinic anhydride, such as dibromosuccinic anhydride), acyl azide, p-diazoiumbenzyl ether, 3-(p-diazoniumphenoxy)-2-hydroxypropylether) and the like. The activated polymer is then reacted with a decoy polypeptide herein to produce a decoy polypeptide derivatized with a polymer. Alternatively, a functional group in the decoy polypeptide provided herein can be activated for reaction with the polymer, or the two groups can be joined in a concerted coupling reaction using known coupling methods. It will be readily appreciated that the decoy polypeptide be derivatized with PEG using a myriad of other reaction schemes known to and used by those of skill in the art

### Methods of Making Decoy Polypeptides

Also provided are isolated nucleic acids encoding the decoy polypeptides described herein, vectors comprising such nucleic acids, and host cells comprising such vectors or nucleic acids. An “isolated” nucleic acid molecule is a nucleic acid molecule that is identified and separated from at least one contaminant. In some embodiments, a decoy polypeptides described herein is produced using recombinant techniques. For example, the nucleic acid(s) encoding a decoy polypeptide may be inserted into a replicable vector for further cloning (e.g., amplification of the DNA) or for expression. DNA encoding a decoy polypeptide can be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. In some embodiments, a decoy polypeptide described herein may be produced as a fusion with a heterologous or homologous polypeptide. The heterologous or homologous polypeptide may include, e.g., a signal sequence and/or a protease cleavage site at the N-terminus of the mature protein, etc. In some embodiments, a heterologous signal sequence that is recognized and processed (i.e., cleaved by a signal peptidase) by the host cell may be selected. For prokaryotic host cells that do not recognize and process an eukaryotic signal sequence, the signal sequence is substituted by a prokaryotic signal sequence.

Examples of suitable host cells for cloning or expressing nucleic acids provided herein include, but are not limited to, e.g., prokaryotic cells, microbial cells (such as yeast cells), insect cells, or eukaryotic cells (such as mammalian cells). Examples of useful mammalian host cell lines are, e.g., monkey kidney CV1 line transformed by SV40 (COS-7, ATCC CRL 1651); human embryonic kidney line (293 or 293 cells subcloned for growth in suspension culture, Graham et al., J. Gen Viral. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1.982)); MRC 5 cells; FS4 cells; and a human hepatoma line (Hep G2). Host cells are transformed with the above-described expression or cloning vectors for decoy polypeptide production and cultured in conventional nutrient media modified as appropriate for inducing promoters, selecting transformants, or amplifying the genes encoding the desired sequences.

Decoy polypeptides expressed by host cells may be purified using chromatography techniques known in the art. Exemplary techniques that can be used to purify a decoy polypeptide include, for example, hydroxylapatite chromatography, mixed mode chromatography, anion and/or cation exchange chromatography, gel electrophoresis, dialysis, and affinity chromatography (such as protein A, protein L, and/or protein G chromatography). The suitability of protein A as an affinity ligand depends on the isotype of the immunoglobulin Fc domain that is present in the decoy polypeptide. Protein A can be used to purify antibodies that are based on human γ1, γ2, or γ4 heavy chains (Lindmark et al., J. Immunol. Meth. 62:1-13 (1983)). Protein G is usually recommended for human IgG3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the decoy polypeptide comprises a CH3 domain, the BakerbondABX™ resin (J. T. Baker, Phillipsburg, N.J.) may be useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also well known and widely used.

Following any preliminary purification step(s), the mixture comprising the decoy polypeptide and contaminants may be subjected to low pH hydrophobic interaction chromatography using an elution buffer at a pH between about 2.5-4.5, preferably performed at low salt concentrations (e.g., from about 0-0.25M salt).

### Methods of Detecting, Imaging, and Visualizing

Also provided herein are methods for detecting, imaging, or visualizing a cell expressing CD47 comprising contacting a population of cells with a decoy polypeptide described herein comprising a detectable label. In some embodiments, the detectable label comprises an enzymatic label such as horseradish peroxidase (HRP), alkaline phosphatase (AP) or glucose oxidase. In additional aspects, the detectable label comprises a fluorescent label such as Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, BODIPY® FL, Coumarin, Cy®3, Cy®5, Fluorescein (FITC), Oregon Green®, Pacific Blue™, Pacific Green™, Pacific Orange™, Tetramethylrhodamine (TRITC), Texas Red® or other fluorescent label. In further aspects, the detectable label comprises a radioactive isotope such as 32P, 33P, 3H, 14C, 125I or other radioactive isotope.

In some embodiments the methods include detecting, imaging, or visualizing a tumor cell, a virally infected cell, a bacterially infected cell, an autoreactive T cell, damaged red blood cells, arterial plaques, or fibrotic tissue. In some embodiments, the cell is a healthy normal cell such as hematopoietic stem cell, a healthy myeloid or lymphoid precursor cell, or a healthy differentiated hematopoietic cell type such as a T cell, a B cell, a plasma cell, or an NK cell. In some embodiments, the methods comprise detecting, imaging, or visualizing a cell in vivo, ex vivo, or in vitro. In some embodiments, the cell or tissue is imaged or visualized via microscopy, fluorescent microscopy, fluorescence activated cell sorting or positron emission tomography (PET) imaging. In some embodiments, the method is a diagnostic.

### Methods of Treatment

Provided herein are methods for stimulating the immune system of a subject in need thereof, which methods comprise administering a decoy polypeptide described herein. In some instances, the administration of decoy polypeptide described herein induces and/or sustains phagocytosis of a cell expressing CD47. In other instances, the administration of a decoy polypeptide described herein induces and/or sustains phagocytosis of a cell not expressing CD47. In some instances, the cell is a cancer cell, a virally infected cell, a bacterially infected cell, an autoreactive T or B cell, a damaged red blood cell, an arterial plaque, or a cell in fibrotic tissue.

Also provided herein are methods of treating of cancer in a subject comprising administering a decoy polypeptide described herein to the subject. In some embodiments, the subject has cancer and/or has been diagnosed with cancer. In some embodiments, the subject is suspected of having cancer. In some embodiments, the cancer is selected from the group consisting of breast cancer, lung cancer, adenocarcinoma of the lung, squamous cell lung cancer, small cell lung cancer, non-small cell lung cancer, head and neck cancer, brain tumor or brain cancer, abdominal cancer, colon cancer, rectal cancer, colorectal cancer, esophageal cancer, parapharyngeal cancer, gastrointestinal cancer, stomach cancer, gastric cancer, gastrointestinal stromal tumor cancer, glioma, liver cancer, oral cancer, tongue cancer, neuroblastoma, osteosarcoma, ovarian cancer, renal cancer, renal cell cancer, renal pelvis cancer, bladder cancer, urinary bladder cancer, urinary tract cancer, pancreatic cancer, retinoblastoma, cervical cancer, uterine cancer, oropharyngeal cancer, bronchus cancer, Merkel cell carcinoma, virally induced cancer, prostate cancer, Wilm's tumor, multiple myeloma, skin cancer (including melanoma and non-melanoma skin cancer), lymphoma, leukemia, blood cancer, thyroid cancer, bone cancer, adenocystic tumor, chondrosarcoma, pancreatic islet cell tumor, neuroendocrine tumor, prostate cancer, ovarian cancer, glioblastoma, endometrial carcinoma, endometrial cancer, leiomyosarcoma, gall bladder cancer, hepatocellular cancer, hematological cancer, multiple myeloma, acute myelogenous leukemia (also known as acute myeloid leukemia), acute/chronic lymphoblastic leukemia, hairy-cell leukemia, follicular lymphoma, multiple myeloma, plasmacytoma, diffuse large B-cell lymphoma. In some embodiments, the cancer is a hematological cancer. In some embodiments, the cancer is multiple myeloma, acute/chronic myelogenous leukemia, acute/chronic lymphoblastic leukemia, hairy-cell leukemia, follicular lymphoma, multiple myeloma, plasmacytoma or diffuse large B-cell lymphoma.

In some embodiments, the cancer is associated with expression of CD47 including but not limited to Acute myeloid leukemia (AML), Acute leukocytic leukemia (ALL), Hodgkin's lymphoma (HL), Non-Hodgkin's B cell lymphoma (NHBCL), Chronic leukocytic leukemia (B-CLL), Multiple myeloma (MM), pancreatic adenocarcinoma, pancreatic neuroendocrine tumor (PanNET), glioma, medulloblastoma, astrocytoma, prostate cancer, osteosarcoma, small cell lung carcinoma (SCLC), non-small cell lung carcinoma (NSCLC), melanoma, squamous cell head and neck carcinoma, prostate carcinoma, ovarian cancer, breast cancer, colon cancer, renal cancer, and bladder cancer. In some embodiments, the cancer is associated with solid tumors. In certain instances, the solid tumors are advanced, e.g., stage 3 or 4. In some embodiments, the solid tumors are histologically associated with the expression of the CD47.

In some embodiments, “treatment” or “treating” or “treated” refers to therapeutic treatment wherein the object is to slow (lessen) an undesired physiological condition, disorder or disease, or to obtain beneficial or desired clinical results. In some embodiments, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. In some embodiments, treatment includes eliciting a clinically significant response without excessive levels of side effects. In some embodiments, treatment includes prolonging survival as compared to expected survival if not receiving treatment. In some embodiments, “treatment” or “treating” or “treated” refers to prophylactic measures, wherein the object is to delay onset of or reduce severity of an undesired physiological condition, disorder or disease, such as, for example is a subject who is predisposed to a disease (e.g., a subject who carries a genetic marker for a disease such as breast cancer).

The methods of treatment described herein are used for the treatment various stages of cancer, including stages which are locally advanced, metastatic and/or recurrent. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV). Cancer designated as recurrent generally is defined as the cancer has recurred, usually after a period of time, after being in remission or after a tumor has visibly been eliminated. Recurrence can either be local, i.e., appearing in the same location as the original, or distant, i.e., appearing in a different part of the body. In some embodiments, a cancer treatable by combination therapies described herein is unresectable, or unable to be removed by surgery.

Exemplary Combination Treatments

In some embodiments, the methods of treatment described herein provide adjunct therapy to any other cancer therapy prescribed to a subject. In some embodiments, a method of treatment comprises administering a decoy polypeptide described herein in combination with at least one additional anti-cancer agent (e.g., at least two, at least three, or at least four additional anti-cancer agents) including, but not limited to, for example, methotrexate (RHEUMATREX®, Amethopterin), cyclophosphamide (CYTOXAN®), abiraterone, abemaciclib, altretamine, thalidomide (THALIDOMID®), acridine carboxamide, Actimid®, actinomycin, actinomycin-D, afatinib, 17-N-allylamino-17-demethoxygeldanamycin, alectinib, alpelisib, aminopterin, amsacrine, anlotinib, anthracycline, antineoplastic, antineoplaston, apartinib, 5-azacytidine, 6-mercaptopurine, 6-thioguanine, arabinosylcytosine, axitinib, azacitidine, azathioprine, BL22, bendamustine, binimetinib, biricodar, bleomycin, bortezomib, bosutinib, brigatinib, bryostatin, busulfan, cabozantinib, calyculin, camptothecin, capecitabine, carboplatin, carmustine, ceritinib, chlorambucil, cisplatin, cladribine, clofarabine, cobimetinib, crizotinib, cytarabine, dabrafenib, dacarbazine, dacomitinib, dasatinib, daunorubicin, decitabine, dexamethasone, dichloroacetic acid, discodermolide, docetaxel, doxorubicin, encorafenib, epirubicin, entrectinib, enzalutamide, epothilone, erdafitinib, eribulin, erlotinib, estramustine, etoposide, everolimus, exatecan, exisulind, ferruginol, floxuridine, fludarabine, fluorouracil (such as 5-fluorouracil), folinic acid, fosfestrol, fotemustine, fruquintinib, ganciclovir, gefitinib, gemcitabine, gilteritinib, goserelin, hexamethylmelamine, hydroxycarbamide, hydroxyurea, IT-101, ibrutinib, icotinib, idarubicin, idelalisib, ifosfamide, imatinib, irinoimiquimod, irinotecan, irofulven, ivosidenib, ixabepilone, laniquidar, lapatinib, larotrectinib, lenalidomide, lenvatinib, lorlatinib, lomustine, lurtotecan, mafosfamide, masoprocol, mechlorethamine, melphalan, mercaptopurine, methotrexate, methylprednisolone, mitomycin, mitotane, mitoxantrone, nelarabine, neratinib, niraparib, nilotinib, nintedanib, oblimersen, olaparib, osimertinib, oxaliplatin, nedaplatin, phenanthriplatin, picoplatin, PAC-I, paclitaxel, palbociclib, pazopanib, pemetrexed, pegfilgrastim, pentostatin, pipobroman, pixantrone, plicamycin, prednisone, ponatinib, procarbazine, proteasome inhibitors (e.g., bortezomib), pyrotinib, raltitrexed, rebeccamycin, Revlimid®, regorafenib, ribociclib, rubitecan, rucaparib, ruxolitinib, SN-38, salinosporamide A, satraplatin, sirolimus, sonidegib, sorafenib, streptozocin, streptozotocin, sunitinib, swainsonine, talazoparib, tariquidar, taxane, tegafur-uracil, temsirolimus, teniposide, temozolomide, testolactone, thioTEPA, tioguanine, topotecan, trabectedin, trametinib, tretinoin, trifluridine, triplatin tetranitrate, tris(2-chloroethyl)amine, troxacitabine, uracil mustard, valrubicin, vandetanib, vemurafenib, venetoclax (ABT-199), navitoclax (ABT-263), vinblastine, vincristine, vinorelbine, vismodegib, vorinostat, ziv-aflibercept (ZALTRAP®), zosuquidar, or the like.

In some embodiments, a method of treatment comprises administering a decoy polypeptide described herein in combination with anti-cancer agents/chemotherapeutic agents of a particular class. For example, in some embodiments, a method of treatment comprises administering a decoy polypeptide described herein in combination with an adrenal inhibitor (including, but not limited to adrenal inhibitors described herein). For example, in some embodiments, a method of treatment comprises administering a decoy polypeptide described herein in combination with an anthracycline (including, but not limited to anthracyclines described herein). In some embodiments, a method of treatment comprises administering a decoy polypeptide described herein in combination with an alkylating agent (including, but not limited to alkylating agents described herein). In some embodiments, a method of treatment comprises administering a decoy polypeptide described herein in combination with an androgen inhibitor (including, but not limited to androgen inhibitors described herein). In some embodiments, a method of treatment comprises administering a decoy polypeptide described herein in combination with an antimetabolite, e.g., a purine analog, (including, but not limited to antimetabolites, e.g., purine analogs, described herein). In some embodiments, a method of treatment comprises administering a decoy polypeptide described herein in combination with an antitumor antibiotic (including, but not limited to antitumor antibiotics described herein). In some embodiments, a method of treatment comprises administering a decoy polypeptide described herein in combination with a BLC-2 inhibitor (including, but not limited to BLC-2 inhibitors described herein). In some embodiments, a method of treatment comprises administering a decoy polypeptide described herein in combination with a BTK inhibitor (including, but not limited to BTK inhibitors described herein). In some embodiments, a method of treatment comprises administering a decoy polypeptide described herein in combination with a CDK 4/6 inhibitor (including, but not limited to CDK 4/6 inhibitors described herein). In some embodiments, a method of treatment comprises administering a decoy polypeptide described herein in combination with a colony stimulating factor (including, but not limited to colony stimulating factors described herein). In some embodiments, a method of treatment comprises administering a decoy polypeptide described herein in combination with a corticosteroid (including, but not limited to corticosteroids described herein). In some embodiments, a method of treatment comprises administering a decoy polypeptide described herein in combination with an EGFR inhibitor (including, but not limited to EGFR inhibitors described herein). In some embodiments, a method of treatment comprises administering a decoy polypeptide described herein in combination with a gonadotrophin releasing hormone (GnRH) agonist (including, but not limited to GnRH agonists described herein). In some embodiments, a method of treatment comprises administering a decoy polypeptide described herein in combination with a mitotic inhibitor/microtubule inhibitor (including, but not limited to mitotic inhibitors/microtubule inhibitors described herein). In some embodiments, a method of treatment comprises administering a decoy polypeptide described herein in combination with an mTOR kinase inhibitor (including, but not limited to mTOR kinase inhibitors described herein). In some embodiments, a method of treatment comprises administering a decoy polypeptide described herein in combination with a proteasome inhibitor (including, but not limited to proteasome inhibitors described herein). In some embodiments, a method of treatment comprises administering a decoy polypeptide described herein in combination with a signal transduction inhibitor, e.g., a protein-tyrosine kinase inhibitor, a PAK4 inhibitor, a PI3K inhibitor, (including, but not limited to signal transduction inhibitors described herein). In some embodiments, a method of treatment comprises administering a decoy polypeptide described herein in combination with a topoisomerase inhibitor, (including, but not limited to topoisomerase inhibitors described herein). In some embodiments, a method of treatment comprises administering a decoy polypeptide described herein in combination with a tyrosine kinase inhibitor, (including, but not limited to tyrosine kinase inhibitors described herein). In some embodiments, a method of treatment comprises administering a decoy polypeptide described herein in combination with a VEGF inhibitor, such as a VEGF1 inhibitor, a VEGF2 inhibitor, and/or a VEGF3 inhibitor (including, but not limited to VEGF inhibitors described herein). In some embodiments, a method of treatment comprises administering a decoy polypeptide described herein in combination with an agent that modulates apoptosis, e.g., by modulating the activity of Bcl-2, Mcl1, Bcl-lx, etc., (including, but not limited to agents that modulate apoptosis, e.g., by modulating the activity of Bcl-2, Mcl1, Bcl-lx, etc., described herein). In some embodiments, a method of treatment comprises administering a decoy polypeptide described herein in combination with a platinum-based agent, (including, but not limited to platinum-based agents described herein). In some embodiments, a method of treatment comprises administering a decoy polypeptide described herein in combination with an inhibitor of NTRK1, NTRK2, and/or NTRK3, an ALK inhibitor, a ROS inhibitor, a FLT3 inhibitor, a BRAF inhibitor, an inhibitor of MEK1 and/or MEK2, an inhibitor of HER2, HER3, and/or HER 4, an inhibitor of RET/PTC, an inhibitor of BCR-ABL, a c-KIT inhibitor, an inhibitor of PDGFR-alpha and/or PDGFR-beta, an inhibitor of FGFR1, FGFR2, FGFR3, and/or FGFR4, an Smoothened inhibitor and/or an inhibitor of PARP1, PARP2, and/or PARP3 (including, but not limited to inhibitors described herein).

In some embodiments, the methods of treatment described herein, a decoy polypeptide is administered in combination with one or more monoclonal antibodies, including, but not limited to, e.g., 3F8, 8H9, Abagovomab, Abciximab, Abituzumab, Abrilumab, Actoxumab, Adalimumab, Adecatumumab, Aducanumab, Afelimomab, Afutuzumab, Alacizumab pegol, ALD518, Alemtuzumab, Alirocumab, Altumomab pentetate, Amatuximab, Anatumomab mafenatox, Anetumab ravtansine, Anifrolumab, Anrukinzumab (IMA-638), Apolizumab, Arcitumomab, Ascrinvacumab, Aselizumab, Atezolizumab, Atinumab, Atlizumab (tocilizumab), Atorolimumab, Avelumab, MSBBapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Blinatumomab, Blosozumab, Bococizumab, Brentuximab vedotin, Briakinumab, Brodalumab, Brolucizumab, Brontictuzumab, Cabiralizumab (FPA008), Camrelizumab, Canakinumab, Cantuzumab mertansine, Cantuzumab ravtansine, Caplacizumab, Capromab pendetide, Carlumab, Catumaxomab, cBR96-doxorubicin immunoconjugate, CC49, Cedelizumab, Certolizumab pegol, Cetuximab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, Crenezumab, CR6261, Dacetuzumab, Daclizumab, Dalotuzumab, Dapirolizumab pegol, Daratumumab, Dectrekumab, Demcizumab, Denintuzumab mafodotin, Denosumab, Derlotuximab biotin, Detumomab, Dinutuximab, Diridavumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab (RG7155), Emibetuzumab, Enavatuzumab, Enfortumab vedotin, Enlimomab pegol, Enoblituzumab, Enokizumab, Enoticumab, Ensituximab, Epitumomab cituxetan, Epratuzumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Infliximab, Intetumumab, Inolimomab, Inotuzumab ozogamicin, Ipilimumab, Iratumumab, Isatuximab, Itolizumab, Ixekizumab, Keliximab, Labetuzumab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, MSB0010718C (avelumab), Mapatumumab, Margetuximab, Maslimomab, Mavrilimumab, Matuzumab, MEDI6469, MEDI0680, MED16383, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Rituximab, Robatumumab, Roledumab, Romosozumab, Rontalizumab, Rovelizumab, Ruplizumab, Sacituzumab govitecan, Samalizumab, SAR650984 (Isatuximab) Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Sintilimab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teplizumab, Teprotumumab, Tesidolumab, TGN1412, Ticilimumab (tremelimumab), Tildrakizumab, Tigatuzumab, TNX-650, Tocilizumab (atlizumab), Toralizumab, Toripalimab, Tosatoxumab, Tositumomab, Tovetumab, Tralokinumab, Trastuzumab, trastuzumab-emtansine, TRBS07, Tregalizumab, Tremelimumab, Tucotuzumab celmoleukin, Tuvirumab, Ublituximab, Ulocuplumab, Urelumab, Urtoxazumab, Ustekinumab, Utomilumab (PF-05082566), Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Volociximab, Vonlerolizumab (RG7888), Vorsetuzumab mafodotin, Votumumab, Zalutumumab, Zanolimumab, Zatuximab, Ziralimumab, or Zolimomab aritox, including biosimilars of any of the preceding therapeutic antibodies. In some embodiments, the decoy polypeptide is administered in combination with one or more monoclonal antibodies including, but not limited to, e.g., an anti-CD20 antibody, an anti-EGFR antibody, an anti-Her2/Neu (ERBB2) antibody, an anti-EPCAM antibody, an anti-GL2 antibody, anti-GD2, anti-GD3, anti-CD2, anti-CD3, anti-CD4, anti-CD8, anti-CD I 9, anti-CD22, anti-CD30, anti-CD33, anti-CD39, anti-CD45, anti-CD47, anti-CD52, anti-CD56, anti-CD70, anti-CD73, anti-CD117, an anti-SIRPA antibody, an anti-LILRB1, an anti-LILRB2, an anti-LILRB4 antibody, an anti-PD-1 antibody, an anti-PD-L1 antibody, an anti-PD-L2 antibody, or any antibody designed to bind to a tumor cell, a virally- or bacterially-infected cell, immune cell, or healthy normal cell, or to a cytokine, chemokine, or hormone of any kind.

In some embodiments, a decoy polypeptide described herein is administered in combination with one or more monoclonal antibodies targeting, e.g., CS1/SLAMF7, Trop-2, VWF, vimentin, VEGFR2, VEGFR-1, VEGF, VEGF-A, TYRP1 (glycoprotein 75), TWEAK receptor, tumor specific glycosylation of MUC1, tumor antigen CTAA16.88, TRAIL-R2, TRAIL-R1, TNF-alpha, TGF-beta, TGF beta 2, TGF beta 1, TFPI, tenascin C, TEM1, TAG-72, T-cell receptor, STEAP1, sphingosine-1-phosphate, SOST, SLAMF7, BCL-2, selectin P, SDC1, sclerostin, RTN4, RON, Rhesus factor, RHD, respiratory syncytial virus, RANKL, rabies virus glycoprotein, platelet-derived growth factor receptor beta, phosphatidylserine, phosphate-sodium co-transporter, PDGF-R alpha, PDCD1, PD-1, PD-L1, PCSK9, oxLDL, OX-40, NRP1, Notch receptor 4, Notch receptor 3, Notch receptor 2, Notch receptor 1, NOGO-A, NGF, neural apoptosis-regulated proteinase 1, NCA-90 (granulocyte antigen), NARP-1, N-glycolylneuraminic acid, myostatin, myelin-associated glycoprotein, mucin CanAg, MUC1, MSLN, MS4A1, MIF, mesothelin, MCP-1, LTA, LOXL2, lipoteichoic acid, LINGO-1, LFA-1 (CD11a), Lewis-Y antigen, L-selectin (CD62L), KIR2D, ITGB2 (CD18), ITGA2, interferon alpha/beta receptor, interferon receptor, interferon gamma-induced protein, integrin αvβ3, integrin αIIβ3, integrin α7β7, integrin α5β1, integrin α4β7, integrin α4, insulin-like growth factor I receptor, Influenza A hemagglutinin, ILGF2, IL9, IL6, IL4, IL3 IRA, IL23, ILI 7A, IL-6 receptor, IL-6, IL-S, IL-4, IL-23, IL-22, IL-I, IL-I 7A, IL-I 7, IL-13, IL-I2, IL-I, IL 20, IGHE, IgG4, IGF-I, IGF-I receptor, IgE Fc region, IFN-gamma, IFN-alpha, ICAM-1 (CD54), human TNF, human scatter factor receptor kinase, Hsp90, HNGF, HLA-DR, HIV-1, histone complex, HHGFR, HGF, HER3, HER2, HER2/neu, HER1, hepatitis B surface antigen, hemagglutinin, GUCY2C, GPNMB, GMCSF receptor alpha-chain, glypican 3, GD3 ganglioside, GD2, ganglioside GD2, Frizzled receptor, folate receptor 1, folate hydrolase, fibronectin extra domain-B, fibrin II, beta chain, FAP, F protein of respiratory syncytial virus, ERBB3, episialin, EpCAM, endotoxin, EGFR, EGFL7, E. coli shiga toxin type-2, E. coli shiga toxin type-I, DRS, DPP4, DLL4, dabigatran, cytomegalovirus glycoprotein B, CTLA-4, CSF2, CSF1R, clumping factor A, CLDN18.2, ch4DS, CFD, CEA-related antigen, CEA, CD80, CD79B, CD74, CD73, CD70, CD6, CD56, CD52, CD51, CD5, CD44 v6, CD41, CD40 ligand, CD40, CD4, CD39, CD38, CD37, CD33, CD30 (TNFRSF8), CD123, CD138, CD3 epsilon, CD3, CD28, CD274, CD27, CD2S (a chain of IL-2 receptor), CD23 (IgE receptor), CD221, CD22, CD200, CD20, CD2, CD19, CD137, CD154, CD152, CD15, CD147 (basigin), CD140a, CD125, CD11, CD-18, CCR5, CCR4, CCL11 (eotaxin-I), cardiac myosin, carbonic anhydrase 9 (CA-IX), Canis lupus familiaris IL31, CA-125, C5, C242 antigen, C-X-C chemokine receptor type 4, beta-amyloid, BAFF, B7-H3, B-lymphoma cell, AOC3 (VAP-I), anthrax toxin, protective antigen, angiopoietin 3, angiopoietin 2, alpha-fetoprotein, AGS-22M6, adenocarcinoma antigen, ACVR2B, activin receptor-like kinase I, 5T4, 5AC, 4-IBB or 1-40-beta-amyloid.

In some embodiments, a decoy polypeptide described herein is administered in combination with a second antibody, e.g., an antibody that binds an antigen expressed by the cancer (e.g., an effective amount of the second antibody, which in some embodiments as described above may be considered in the context of administering an anti-SIRP-α antibody of the present disclosure). Exemplary antigens expressed by cancers are known in the art and include without limitation EphA4, BCMA, Mucin 1, Mucin 16, PTK7, PD-L1, STEAP1, Endothelin B Receptor, mesothelin, EGFRvIII, ENPP3, SLC44A4, GNMB, nectin 4, NaPi2b, LIV-1A, Guanylyl cyclase C, DLL3, EGFR, HER2, VEGF, VEGFR, integrin αVβ3, integrin α5β1, MET, IGF1R, TRAILR1, TRAILR2, RANKL, FAP, Tenascin, Le^{y}, EpCAM, CEA, gpA33, PSMA, TAG72, a mucin, CAIX, EPHA3, folate receptor α, GD2, GD3, and an MHC/peptide complex comprising a peptide from NY-ESO-1/LAGE, SSX-2, a MAGE family protein, MAGE-A3, gp100/pmel17, Melan-A/MART1, gp75/TRP1, tyrosinase, TRP2, CEA, PSA, TAG-72, immature laminin receptor, MOK/RAGE-1, WT-1, SAP-1, BING-4, EpCAM, MUC1, PRAME, survivin, BRCA1, BRCA2, CDK4, CML66, MART-2, p53, Ras, β-catenin, TGF-βRII, HPV E6, or HPV E7. For example, in some embodiments, an antibody of the present disclosure is administered in combination with a monoclonal antibody that binds CD123 (also known as IL-3 receptor alpha), such as talacotuzumab (also known as CSL362 and JNJ-56022473).

In some embodiments, a decoy polypeptide described herein is administered in combination with a second antibody that binds an antigen expressed by an NK cell. Exemplary antigens expressed by an NK cell include, without limitation, NKR-PIA (KLRB1), CD94 (NKG2A), KLRG1, KIR2DL5A, KIR2DL5B, KIR2DL1, KIR2DL2, KIR2DL3, KIR2DS2, KIR2DS3, KIR2DS4, KIR2DS5, KIR3DS1, KIR2DS1, CD94 (NKG2C/E), NKG2D, CD160 (BY55), CD16 (FcγRIIIA), NKp46 (NCR1), NKp30 (NCR3), NKp44 (NCR2), DNAM1 (CD226), CRTAM, CD27, NTB-A (SLAMF6), PSGL1, CD96 (Tactile), CD100 (SEMA4D), NKp80 (KLRF1, CLEC5C), SLAMF7 (CRACC, CS1, CD319), and CD244 (2B4, SLAMF4).

In some embodiments, a decoy polypeptide described herein is administered in combination with an immunotherapeutic agent. An immunotherapeutic agent may refer to any therapeutic that targets the immune system and promotes a therapeutic redirection of the immune system, such as a modulator of a costimulatory pathway, cancer vaccine, recombinantly modified immune cell, etc. Exemplary and non-limiting immunotherapeutic agents are described infra. Without wishing to be bound to theory, it is thought that the decoy polypeptides of the present disclosure are suitable for use with immunotherapeutic agents due to complementary mechanisms of action, e.g., in activating both macrophages and other immune cells such as T_{effector} cells to target tumor cells. In some embodiments, the immunotherapeutic agent is or comprises an antibody. Exemplary antigens of immunotherapeutic antibodies are known in the art and include without limitation BDCA2, BDCA4, ILT7, LILRB1, LILRB2, LILRB3, LILRB4, LILRB5, Siglec-3, Siglec-7, Siglec-9, Siglec-10, Siglec-15, FGL-1, CD200, CD200R, CSF-1R, CD24, CD40, CD40L, CD163, CD206, DEC205, CD47, CD123, arginase, IDO, TDO, AhR, EP2, COX-2, CCR2, CCR-7, CXCR1, CX₃CR1, CXCR2, CXCR3, CXCR4, CXCR7, TGF-β RI, TGF-β RII, c-Kit, CD244, L-selectin/CD62L, CD11b, CD11c, CD68, 41BB, CTLA4, PD1, PD-L1, PD-L2, TIM-3, BTLA, VISTA, LAG-3, CD28, OX40, GITR, CD137, CD27, HVEM, CCR4, CD25, CD103, KIrg1, Nrp1, CD278, Gpr83, TIGIT, CD154, CD160, TNFR2, PVRIG, DNAM, and ICOS. Immunotherapeutic agents that are approved or in late-stage clinical testing include, without limitation, ipilimumab, pembrolizumab, nivolumab, atezolizumab, avelumab, durvalumab, and the like. In certain embodiments, the decoy polypeptides of the present disclosure is administered in combination with an inhibitor of the PD-L1/PD-1 pathway, e.g., an anti-PD-L1 or anti-PD-1 antibody. As demonstrated herein, combined administration of a decoy polypeptides of the present disclosure and an inhibitor of the PD-L1/PD-1 pathway can result in synergistic anti-tumor activity. In some embodiments, the immunotherapeutic agent is or comprises a vaccine, oncolytic virus, adoptive cell therapy, cytokine, or small molecule immunotherapeutic agent. Examples of such immunotherapeutic agents are known in the art. For example, adoptive cell therapies and therapeutics can include without limitation chimeric antigen receptor T-cell therapy (CAR-T), tumor infiltrating lymphocytes (TILs), TCR engineered T cells, TCR engineered NK cell, and macrophage cell products. Vaccines can include without limitation polynucleotide vaccines, polypeptide vaccines, or cell-based (e.g., tumor or dendritic cell-based) vaccines. Various cytokines useful for the treatment of cancer are known and include without limitation IL-2, IL-15, IL-7, IL-10, IL-12, IL21, TNFα, IFNs, GM-CSF, and engineered cytokine mutants. Small molecule immunotherapeutic agents can include without limitation IDO/TDO inhibitors, AhR inhibitors, arginase inhibitors, A2a R inhibitors, TLR agonists, STING agonists, and Rig-1 agonists.

In some embodiments, a decoy polypeptide described herein is administered in combination with a chemotherapeutic agent or small molecule anti-cancer agent. In some embodiments, the decoy polypeptides of the present disclosure is administered in combination with an immunotherapeutic agent and a chemotherapeutic agent or small molecule anti-cancer agent. For example, it is thought that kinase inhibitors or other inhibitors of signaling pathways (e.g., PAK4, PI3K, mTOR etc.) may be useful in combination with modulation of the immune system for treating cancer. As such, the decoy polypeptides of the present disclosure may find use in combination with one or more chemotherapeutic agents and/or small molecules (e.g., kinase inhibitors) for treating cancer. In some embodiments, the targeted small molecule inhibitor is a VEGFR and/or PDGFR inhibitor, EGFR inhibitor, ALK inhibitor, CDK4/6 inhibitor, PARP inhibitor, mTOR inhibitor, KRAS inhibitor, TRK inhibitor, BCL2 inhibitor, B-raf inhibitor, IDH inhibitor, PI3K inhibitor, DDR (DNA damage response) inhibitor, or hypomethylation agent. In other cases, the targeted small molecule modulates a cellular signaling pathway of the cell expressing CD47, e.g., an IDO/TDO inhibitor, AhR inhibitor, arginase inhibitor, A2a R inhibitor, TLR agonists, STING agonist, or Rig-1 agonist.

In some embodiments, a decoy polypeptide described herein is administered in combination with at least two additional agents (such as anti-cancer agents). In some embodiments, the at a least two additional agents (e.g., anti-cancer agents) are from different classes and/or exert their anti-cancer effects via different mechanisms of action. For example, in some embodiments, a decoy polypeptide described herein is administered in combination with a chemotherapeutic agent (including, but not limited to those described herein) and a therapeutic antibody (including, but not limited to those described herein, e.g., an anti-HER2 antibody). In some embodiments, a decoy polypeptide described herein is administered in combination with a chemotherapeutic agent (including, but not limited to those described herein) and a small molecule inhibitor (including, but not limited to those described herein). Other combinations are also contemplated.

In some embodiments, a decoy polypeptide described herein is administered in combination with a second therapy. In some embodiments, the second therapy is radiotherapy (e.g., gamma-rays, X-rays, and/or the directed delivery of radioisotopes to tumor cells, microwaves, UV radiation, or gene therapy. For example, therapeutic genes include an antisense version of an inducer of cellular proliferation (oncogene), an inhibitor of cellular proliferation (tumor suppressor), or an inducer of programmed cell death (pro-apoptotic gene). In some embodiments, the combination therapies described herein are administered in combination with a surgery (e.g., resection).

In some embodiments, a decoy polypeptide described herein is administered in combination with one or more agents including, without limitation, e.g., anti-diarrheal agents, anti-emetic agents, analgesics, opioids and/or non-steroidal anti-inflammatory agents.

In some embodiments, a decoy polypeptide described herein is administered to a subject who has been pre-treated with cyclophosphamide, or imitanib, or daclizumab and/or other anti-cancer agent. In some embodiments, a decoy polypeptide described herein is administered to a subject who has not been pre-treated with cyclophosphamide and/or other anti-cancer agent.

In some embodiments, treatment with a decoy polypeptide described herein prolongs lifespan and/or increases survival rates for subjects suffering from cancer. In some embodiments, treatment with a decoy polypeptide described herein improves quality of life for a subject suffering from cancer (e.g., a subject needs a lower dose of an anti-cancer drug that causes side-effects when the subject is treated with a decoy polypeptide described herein).

In some embodiments, treatment with a decoy polypeptide described herein induces and/or sustains phagocytosis or ADCC in a subject. Phagocytosis includes phagocytosis by professional phagocytes (e.g. monocytes, macrophages, neutrophils, dendritic cells or mast cells), non-professional phagocytes (e.g. epithelial cells, endothelial cells, fibroblasts or mesenchymal cells) or both. ADCC includes antibody dependence cell-mediated cytotoxicity by myeloid cells including neutrophils, monocytes, and natural killer cells. Measurement of phagocytosis and ADCC is accomplished by any known method including, for example, fluorescence microscopy or flow cytometry. In some embodiments, treatment with a decoy polypeptide described herein induces and/or enhances antibody-dependent cell-mediated phagocytosis (ADCP) or ADCC of IgE producing B and plasma cells by combining the decoy polypeptide comprising a SIRPγ, SIRPβ1, or SIRPβ2 variant with antibodies against M1 prime or CD38 in a subject with asthma or allergy.

Also provided herein are methods for treating a viral infection, disorder or condition in an individual comprising administering to a subject having a viral infection, disorder or condition a decoy polypeptide described herein. In some embodiments, the viral infection, disorder or condition is chronic. In some embodiments, the viral infection, disorder or condition is acute. In some embodiments, the viral infection, disorder or condition is an Adenoviridae such as, Adenovirus; a Herpesviridae such as Herpes simplex, type 1, Herpes simplex, type 2, Varicella-zoster virus, Epstein-Barr virus, Human cytomegalovirus, or Human herpesvirus, type 8); a Papillomaviridae (such as Human papillomavirus); a Polyomaviridae (such as BK virus or JC virus); a Poxviridae (such as Smallpox); a Hepadnaviridae (such as Hepatitis B virus); a Parvoviridae (such as Human bocavirus or Parvovirus); a Astroviridae (such as Human astrovirus); a Caliciviridae (such as Norwalk virus); a Picomaviridae (such as coxsackievirus, hepatitis A virus, poliovirus, rhinovirus); a Coronaviridae (such as Severe acute respiratory syndrome virus); a Flaviviridae (such as Hepatitis C virus, yellow fever virus, dengue virus, West Nile virus); a Togaviridae (such as Rubella virus); a Hepeviridae (such as Hepatitis E virus); a Retroviridae (such as Human immunodeficiency virus (HIV)); a Orthomyxoviridae (such as Influenza virus); a Arenaviridae (such as Guanarito virus, Junin virus, Lassa virus, Machupo virus, or Sabia virus); a Bunyaviridae (such as Crimean-Congo hemorrhagic fever virus); a Filoviridae (such as Ebola virus or Marburg virus); a Paramyxoviridae (such as Measles virus, Mumps virus, Parainfluenza virus, Respiratory syncytial virus, Human metapneumovirus, Hendra virus, or Nipah virus); a Rhabdoviridae (such as Rabies virus); Hepatitis D virus; or a Reoviridae (such as Rotavirus, Orbivirus, Coltivirus, Banna virus). In particular aspects, the viral infection, disorder or condition is Human immunodeficiency virus (HIV), Human cytomegalovirus, Epstein-Barr virus, Hepatitis C virus, or Hepatitis B virus.

Also provided herein are methods for treating a bacterial infection, disorder or condition in a subject comprising administering to the subject having a bacterial infection, disorder or condition a decoy polypeptide described herein. In some embodiments, the bacterial infection, disorder or condition is chronic. In some embodiments, the bacterial infection, disorder or condition is acute. In some embodiments, the bacterial infection is a Bacillus such as Bacillus anthracis or Bacillus cereus; a Bartonella such as Bartonella henselae or Bartonella quintana; a Bordetella such as Bordetella pertussis; a Borrelia such as Borrelia burgdorferi, Borrelia garinii, Borrelia afzelii, Borrelia recurrentis; a Brucella such as Brucella abortus, a Brucella canis, Brucella melitensis or Brucella suis; a Campylobacter such as Campylobacter jejuni; a Chlamydia or Chlamydophila such as Chlamydia pneumoniae, Chlamydia trachomatis, Chlamydophila psittaci; a Clostridium such as Clostridium botulinum, a Clostridium difficile, Clostridium perfringens, Clostridium tetani; a Corynebacterium such as Corynebacterium diphtheriae; an Enterococcus such as Enterococcus faecalis or Enterococcus faecium; a Escherichia such as Escherichia coli; a Francisella such as Francisella tularensis; a Haemophilus such as Haemophilus influenzae; a Helicobacter such as Helicobacter pylori; a Legionella such as Legionella pneumophila; a Leptospira such as Leptospira interrogans, Leptospira santarosai, Leptospira weilii or Leptospira noguchii; a Listeria such as Listeria monocytogenes; a Mycobacterium such as Mycobacterium leprae, Mycobacterium tuberculosis or Mycobacterium ulcerans; a Mycoplasma such as Mycoplasma pneumoniae; a Neisseria such as Neisseria gonorrhoeae or Neisseria meningitidis; a Pseudomonas such as Pseudomonas aeruginosa; a Rickettsia such as Rickettsia rickettsii; a Salmonella such as Salmonella typhi or Salmonella typhimurium; a Shigella such as Shigella sonnei; a Staphylococcus such as Staphylococcus aureus, Staphylococcus epidermidis, Staphylococcus saprophyticus; a Streptococcus such as Streptococcus agalactiae, Streptococcus pneumoniae, Streptococcus pyogenes; a Treponema such as Treponema pallidum; a Vibrio such as Vibrio cholerae; a Yersinia such as Yersinia pestis, Yersinia enterocolitica or Yersinia pseudotuberculosis.

Also provided herein are methods for treating anemia in a subject comprising administering to the subject a decoy polypeptide described herein. In some embodiments, the anemia is a thalassemia, an aplastic anemia, a haemolytic anemia, a sickle cell anemia, a pernicious anemia or a fanconi anemia.

Also provided herein are methods for treating a person undergoing a transplant comprising administering to a subject undergoing an organ transplant a decoy polypeptide described herein. In some embodiments, the transplanted organ is a heart, a lung, a heart and lung, a kidney, a liver, a pancreas, an intestine, a stomach, a testis, a hand, a cornea, skin, islets of Langerhans, bone marrow, stem cells, blood, a blood vessel, a heart valve, or a bone.

Also provided herein are methods for treating a person with autoimmune disease or inflammatory disorder comprising administering to a subject with autoimmune disease a decoy polypeptide described herein. In some embodiments, the autoimmune disease is an antibody-mediated inflammatory or autoimmune disease, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic, esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type I diabetes), Juvenile myositis, Kawasaki syndrome, Lambert-Eaton syndrome, vasculitis, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, graft versus host disease, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, acute coronary syndrome, ischemic reperfusion, myasthenia gravis, asthma, acute respiratory distress syndrome (ARDS), Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, spondyloarthropathy, Sarcoidosis, Schmidt syndrome, Scleritis, Scleroderma, acute coronary syndrome, Sjogren's syndrome, progressive systemic sclerosis, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type I diabetes, Ulcerative colitis, Undifferentiated connective tissue disease (UCTD), Uveitis, Vasculitis, Vesiculobullous dermatosis, Vitiligo or Wegener's granulomatosis (now termed Granulomatosis with Polyangiitis (GPA)). The diseases described above fall into this category; depletion of autoreactive T or B cells may both be part of a regimen to treat the listed autoimmune diseases.

### Dosages

The dosage of a decoy polypeptide described herein to a subject in need thereof depends on several factors, including, but not limited to, the subject's weight, body surface area, and/or disease state. In some embodiments, the subject to whom the decoy polypeptide is administered is a single organism. In certain embodiments, a decoy polypeptide described herein is administered in combination with subject is a mammal, such as a primate. In some embodiments, the subject is a non-human primate, such as a rhesus or cynomolgous monkey. In some embodiments, the subject is a human. In some embodiments, the subject is a patient, is awaiting medical care or treatment, or is under medical care and treatment.

In some embodiments, the dose of decoy polypeptide administered to a subject is normalized to the body weight of the subject. In some embodiments, a subject is administered a dose of about 10 μg/kg, about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1,000 μg/kg, about 1,100 μg/kg, 1,200 μg/kg, 1,300 μg/kg, 1,400 μg/kg, 1,500 μg/kg, 1,600 μg/kg, 1,700 μg/kg, 1,800 μg/kg, 1,900 μg/kg, about 2,000 μg/kg, about 3000 μg/kg, about 4000 μg/kg, about 5000 μg/kg, about 6000 μg/kg, about 7000 μg/kg, about 8000 μg/kg, about 9000 μg/kg, about 10 mg/kg, about 20 mg/kg, about 30 mg/kg, about 40 mg/kg, about 50 mg/kg, about 60 mg/kg, about 70 mg/kg, about 80 mg/kg about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg of a decoy polypeptide described herein, in either single or cumulative applications. In some embodiments, the dose given to the subject is about 7000 mg/kg of decoy polypeptide per week. In some embodiments, the dose given to the subject is about 70 mg/kg of decoy polypeptide per week. In some embodiments, the dose given to the subject is about 7 mg/kg of decoy polypeptide per week. In some embodiments, the dose given to the subject is about any one of 1,000 μg, 500 μg, 250 μg, 100 μg, or 50 μg of decoy polypeptide per week.

In some embodiments, a subject will receive a dose of the decoy polypeptide described herein, for example, multiple times daily, every day, every other day, once a week, once every other week, once every three weeks, once per month or any other suitable dosing regimen. In some embodiments, a subject will receive a dose of the decoy polypeptide as a continuous infusion. In some embodiments, routinely administering encompasses administering a dose of a decoy polypeptide described herein once a week for a period of time. In some embodiments, the dosing regimen optionally comprises other permutations of decoy polypeptide delivery. In some embodiments, the decoy polypeptide is administered once, twice, three times, four times, five times, six times, or more times a week at a physician's discretion. In some embodiments, a subject is given at least 5 doses over a period of time. In some embodiments, a subject is given greater than or fewer than 5 doses. In some embodiments, a subject is given a dose of about 10 mg/kg of the decoy polypeptide every week. In some embodiments, a subject is given two doses of 5 mg/kg twice a week, or a daily 2 mg/kg dose over five days.

These dosage examples are not limiting and only used to exemplify particular dosing regimens for administering about 10 mg/kg of a decoy polypeptide described herein. For instance, if the appropriate dose for a given situation is 10 mg/kg per week, the doses is optionally broken down into any number of permutations, e.g., four injections of 2.5 mg/kg per week. This also holds true if the appropriate dose for a particular situation is greater than or less than 10 mg/kg.

In some embodiments, the period of time that a decoy polypeptide is administered to the subject is any suitable period as determined by the stage of the disease, the patient's medical history and the attending physician's discretion. Examples of such suitable periods include, but are not limited to, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months or longer. In particular aspects, the treatment period is continued for longer than 24 months, if desired, such as for 30 months, 31 months, 32 months, 33 months, 34 months, 35 months, 36 months, or longer than 36 months. In some embodiments, the period is 6 months, 1 year or 2 years.

In some embodiments, the period of time of dosing for any of the methods described herein is for at least about 2 weeks, at least about 4 weeks, at least about 8 weeks, at least about 16 weeks, at least about 17 weeks, at least about 18 weeks, at least about 19 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 60 weeks, at least about 68 weeks, at least about 72 weeks, at least about 80 weeks, at least about 88 weeks, at least about 96 weeks, or at least about 104 weeks.

In some embodiments, a decoy polypeptide described herein is administered in different phases of treatment. In some embodiments, the decoy polypeptide is administered in both a treatment phase and a maintenance phase. In some embodiments, the treatment phase will comprise administration of the decoy polypeptide formulation in weekly dosages, whereas the maintenance phase is for longer time periods, such as about every 6 weeks, about every 7 weeks, about every 8 weeks, about every 9 weeks, about every 10 weeks, about every 11 weeks, about every 12 weeks, or longer. In some embodiments, the dosage given in the treatment phase will be greater than the dosage given in the maintenance phase. Treatment and maintenance phases are designed to a particular subject so the time and dosages between the treatment and maintenance phases vary from the above examples. Generally, the maintenance phase begins at any time deemed appropriate. In some embodiments, the treatment phase will be eight weeks and the maintenance phase will continue throughout the subject's lifetime. In some embodiments, only a treatment or a maintenance phase will be undertaken.

In some embodiments, a decoy polypeptide described herein is given prophylactically. In some embodiments, the administration of decoy polypeptide prevents onset of disease in a subject (e.g., a subject genetically pre-disposed to developing cancer, such as breast cancer; a subject predisposed to developing a bacterial or viral infection; a subject about to undergo an organ transplant; or a subject predisposed to developing anemia or autoimmune disease.)

The amount of time that a subject should remain on a decoy polypeptide described herein is determined by the attending physician. In some embodiments, it is advantageous to administer the decoy polypeptide for the rest of the subject's lifetime. In some embodiments, a decoy polypeptide is administered in four quadrants of the body, e.g., near lymph nodes, (e.g., in each armpit), in each buttock (e.g., subcutaneously) and the like. In some of such embodiments, a decoy polypeptide is administered via a pump. In some embodiments, a pump and/or delivery device is implanted in a subject to allow chronic dosing. Examples of implantable pumps include and are not limited to Alzet® osmotic pumps

### Kits

Provided herein are kits comprising decoy polypeptides described herein. Such kits comprise a first drug product vial containing a decoy polypeptide and a second vial containing a suitable sterile liquid as described herein for reconstitution. In some embodiments, a kit comprises a first vial, i.e., a drug product vial containing 300 μg of a decoy polypeptide, which represents a 120% fill. This excess is intended to facilitate the withdrawal and administration of the specified dose. In some embodiments, the kit further comprises a second vial containing up to 1 mL of 0.9% sodium chloride solution for injection. After reconstitution of the drug product with 0.6 mL of sodium chloride solution for injection (0.9% w/v), a drug product vial yields 0.5 mL for delivery corresponding to 250 μg of a decoy polypeptide. By way of example, if the dose is mg total, 4 vials are required per dose.

While some embodiments have been shown and described herein, it will be obvious to those skilled in the art that such embodiments are provided by way of example only. Numerous variations, changes, and substitutions will now occur to those skilled in the art without departing from the embodiments described herein. It should be understood that various alternatives to the embodiments described herein may be employed in making and using the decoy polypeptides described above. It is intended that the following claims define the scope of the invention and that methods and structures within the scope of these claims and their equivalents be covered thereby.

### EXAMPLES

### Example 1: Generation of Decoy Polypeptides with Enhanced Binding to Human CD47

The following example describes the design and construction of decoy polypeptides that comprise (a) a SIRPγ d1 domain variant with improved affinity for CD47, a SIRPβ1 d1 domain variant with improved affinity for CD47, or a SIRPβ2 d1 domain variant with improved affinity for CD47 and (b) a human Fc variant with reduced or ablated effector function.

### Materials and Methods

Generation, Expression, and Purification of Decoy Polypeptides

Nucleic acid sequences encoding D1 domain variants of human SIRPα vI (NP_542970.1; SEQ ID NO: 81), human SIRPγ (NP_061026.2; SEQ ID NO: 1), human SIRPβ1 (also known as SIRP beta 1 isoform 1; NP_006056.2; SEQ ID NO: 25), and human SIRPβ2 (also known as SIRP beta 1 isoform 3; Q5TFQ8 SEQ ID NO: 27) comprising specific substitution mutations were synthesized by Genewiz. The amino acid sequences of SEQ ID NOs: 81, 1, 25, and 27 are provided below:

(SEQ ID NO: 81)
EEELQVIQPD KSVLVAAGET ATLRCTATSL IPVGPIQWFR
GAGPGRELIY NQKEGHFPRV TTVSDLTKRN NMDFSIRIGN
ITPADAGTYY CVKFRKGSPD DVEFKSGAGT ELSVRAKPS
(SEQ ID NO: 1)
EEELQMIQPE KLLLVTVGKT ATLHCTVTSL LPVGPVLWFR
GVGPGRELIY NQKEGHFPRV TTVSDLTKRN NMDFSIRISS
ITPADVGTYY CVKFRKGSPE NVEFKSGPGT EMALGAKPS
(SEQ ID NO: 25)
EDELQVIQPE KSVSVAAGES ATLRCAMTSL IPVGPIMWFR
GAGAGRELIY NQKEGHFPRV TTVSELTKRN NLDFSISISN
ITPADAGTYY CVKFRKGSPD DVEFKSGAGT ELSVRAKPS
(SEQ ID NO: 27)
EEELQVIQPD KSISVAAGES ATLHCTVTSL IPVGPIQWFR
GAGPGRELIY NQKEGHFPRV TTVSDLTKRN NMDFSIRISN
ITPADAGTYY CVKFRKGSPD HVEFKSGAGT ELSVRAKPS

The nucleic acids were then fused to a nucleic acid sequence encoding a human IgG-Fc domain with reduced effector function. The decoy polypeptides generated are shown in Table 2. Protein expression constructs were then generated encoding each decoy polypeptide.

TABLE 2
Decoy Polypeptides

| Decoy | SEQ ID | | |
|---|---|---|---|
| polypeptide | NO: | Sequence | Description |
| A | 57 | EEELQIIQPE KLLLVTVGKT ATLHCTITSL FPVGPVLWFR | SIRPγ variant |
| | | GVGPGRVLIY NQRQGPFPRV TTVSDTTKRN NMDFSIRISS | (SEQ ID NO: |
| | | ITPADVGTYY CIKFRKGSPE NVEFKSGPGT EMALGAKPSD | 3) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| B | 58 | EEELQIIQPE KLLLVTVGKT ATLHCTITSL FPVGPIQWFR | SIRPγ variant |
| | | GVGPGRVLIY NQRDGPFPRV TTVSDGTKRN NMDFSIRISS | (SEQ ID NO: |
| | | ITPADVGTYY CVKFRKGTPE DVEFKSGPGT EMALGAKPSD | 4) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| C | 59 | EEELQIIQPE KLLLVTVGKT ATLHCTITSL FPVGPIQWFR | SIRPγ variant |
| | | GVGPGRVLIY NQKDGPFPRV TTVSDGTKRN NMDFSIRISS | (SEQ ID NO: |
| | | ITPADVGTYY CVKFRKGSPE DVEFKSGPGT EMALGAKPSD | 5) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| D | 60 | EEELQIIQPE KLLLVTVGKT ATLHCTITSL FPVGPIQWFR | SIRPγ variant |
| | | GVGPGRVLIY NQKDGHFPRV TTVSDGTKRN NMDFSIRISS | (SEQ ID NO: |
| | | ITPADVGTYY CVKFRKGSPE DVEFKSGPGT EMALGAKPSD | 6) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| E | 61 | EEELQIIQPE KLLLVTVGKT ATLHCTITSL FPVGPIQWFR | SIRPγ variant |
| | | GAGPGRVLIY NQRDGPFPRV TTVSDGTKRN NMDFSIRISS | (SEQ ID NO: |
| | | ITPADVGTYY CIKFRKGTPE DVEFKSGPGT EMALGAKPSD | 7) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| F | 62 | EEELQIIQPE KLLLVTVGKT ATLHCTITSH FPVGPIQWFR | SIRPγ variant |
| | | GVGPGRVLIY NQKDGHFPRV TTVSDGTKRN NMDFSIRISS | (SEQ ID NO: |
| | | ITPADVGTYY CVKFRKGSPE DVEFKSGPGT EMALGAKPSD | 8) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| G | 63 | EEELQIIQPE KLLLVTVGKT ATLHCTITSL FPVGPVLWFR | SIRPγ variant |
| | | GVGPGRVLIY NQRQGPFPRV TTVSDTTKRN NMDFSIRISS | (SEQ ID NO: |
| | | ITPADVGTYY CVKFRKGTPE DVEFKSGPGT EMALGAKPSD | 10) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| H | 64 | EEELQIIQPE KLLLVTVGKT ATLHCTITSL FPVGPIQWFR | SIRPγ variant |
| | | GVGPGRELIY NAREGRFPRV TTVSDLTKRN NMDFSIRISS | (SEQ ID NO: |
| | | ITPADVGTYY CVKFRKGSPE DVEFKSGPGT EMALGAKPSD | 11) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| I | 65 | EEELQIIQPE KLLLVTVGKT ATLHCTITSL FPVGPIQWFR | SIRPγ variant |
| | | GVGPGRVLIY NQREGPFPRV TTVSDGTKRN NMDFSIRISS | (SEQ ID NO: |
| | | ITPADVGTYY CVKFRKGSPE DVEFKSGPGT EMALGAKPSD | 13) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| J | 66 | EEELQIIQPE KLLLVTVGKT ATLHCTITSL LPVGPIQWFR | SIRPγ variant |
| | | GVGPGRELIY NQRDGPFPRV TTVSDGTKRN NMDFSIRISS | (SEQ ID NO: |
| | | ITPADVGTYY CVKFRKGTPE DVEFKSGPGT EMALGAKPSD | 17) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| K | 67 | EEELQIIQPE KLLLVTVGKT ATLHCTLTSL LPVGPILWFR | SIRPγ variant |
| | | GVGPGRVLIY NQRDGPFPRV TTVSDGTKRN NMDFSIRISS | (SEQ ID NO: |
| | | ITPADVGTYY CVKFRKGNPE DVEFKSGPGT EMALGAKPSD | 18) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| L | 68 | EEELQLIQPE KLLLVTVGKT ATLHCTITSL FPPGPIQWFR | SIRPγ variant |
| | | GVGPGRVLIY NQKDGPFPRV TTVSDGTKRN NMDFSIRISS | (SEQ ID NO: |
| | | ITPADVGTYY CVKFRKGIPE DVEFKSGPGT EMALGAKPSD | 19) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| M | 69 | EEELQIIQPE KLLLVTVGKT ATLHCTITSL FPIGPILWFR | SIRPγ variant |
| | | GVGPGRVLIY NQKDGPFPRV TTVSDGTKRN NMDFSIRISS | (SEQ ID NO: |
| | | ITPADVGTYY CVKFRKGSPE DVEFKSGPGT EMALGAKPSD | 21) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| N | 70 | EEELQMIQPE KLLLVTVGKT ATLHCTITSL FPVGPIQWFR | SIRPγ variant |
| | | GAGPGRVLIY NQRDGPFPRV TTVSDGTKRN NMDFSIRISS | (SEQ ID NO: |
| | | ITPADVGTYY CIKFRKGIPE DVEFKSGPGT EMALGAKPSD | 22) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| O | 71 | EEELQMIQPE KLLLVTVGKT ATLHCTVTSL LPVGPVLWFR | SIRPγ variant |
| | | GVGPGRELIY NQKEGHFPRV TTVSDLTKRN NMDFSIRISS | (SEQ ID NO: |
| | | ITPADVGTYY CVKFRKGSPE NVEFKSGPGT EMALGAKPSD | 42) fused to |
| | | KTHTCPPCPA PEAAGAPSVF LFPPKPKDTL MISRTPEVTC | IgG1_AAA_N297A |
| | | VVVDVSHEDP EVKFNWYVDG VEVHNAKTKP REEQYASTYR | (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| P | 72 | EDELQIIQPE KSVSVAAGES ATLRCAITSL FPVGPIQWFR | SIRPβ1 |
| | | GAGAGRVLIY NQRQGPFPRV TTVSETTKRN NLDFSISISN | variant (SEQ |
| | | ITPADAGTYY CIKFRKGSPD DVEFKSGAGT ELSVRAKPSD | ID NO: 26) |
| | | KTHTCPPCPA PEAAGAPSVF LFPPKPKDTL MISRTPEVTC | fused to IgG1 |
| | | VVVDVSHEDP EVKFNWYVDG VEVHNAKTKP REEQYASTYR | AAA N297A |
| | | VVSVLTVLHQ DWLNGKEYKC KVSNKALPAP IEKTISKAKG | (SEQ ID NO: |
| | | QPREPQVYTL PPSREEMTKN QVSLTCLVKG FYPSDIAVEW | 49) |
| | | ESNGQPENNY KTTPPVLDSD GSFFLYSKLT VDKSRWQQGN | |
| | | VFSCSVMHEA LHNHYTQKSL SLSPG | |
| Q | 73 | EEELQIIQPD KSISVAAGES ATLHCTITSL FPVGPIQWFR | SIRPβ2 |
| | | GAGPGRVLIY NQRQGPFPRV TTVSDTTKRN NMDFSIRISN | variant (SEQ |
| | | ITPADAGTYY CIKFRKGSPD DVEFKSGAGT ELSVRAKPSD | ID NO: 28) |
| | | KTHTCPPCPA PEAAGAPSVF LFPPKPKDTL MISRTPEVTC | fused to IgG1 |
| | | VVVDVSHEDP EVKFNWYVDG VEVHNAKTKP REEQYASTYR | AAA N297A |
| | | VVSVLTVLHQ DWLNGKEYKC KVSNKALPAP IEKTISKAKG | (SEQ ID NO: |
| | | QPREPQVYTL PPSREEMTKN QVSLTCLVKG FYPSDIAVEW | 49) |
| | | ESNGQPENNY KTTPPVLDSD GSFFLYSKLT VDKSRWQQGN | |
| | | VFSCSVMHEA LHNHYTQKSL SLSPG | |
| R | 74 | EEELQMIQPE KLLLVTVGKT ATLHCTVTSL LPVGPVLWFR | Wild type |
| | | GVGPGRELIY NQKEGHFPRV TTVSDLTKRN NMDFSIRISS | SIRPγ (SEQ |
| | | ITPADVGTYY CVKFRKGSPE NVEFKSGPGT EMALGAKPSD | ID NO: 1) |
| | | KTHTCPPCPA PEAAGAPSVF LFPPKPKDTL MISRTPEVTC | fused to IgG1 |
| | | VVVDVSHEDP EVKFNWYVDG VEVHNAKTKP REEQYASTYR | AAA N297A |
| | | VVSVLTVLHQ DWLNGKEYKC KVSNKALPAP IEKTISKAKG | (SEQ ID NO: |
| | | QPREPQVYTL PPSREEMTKN QVSLTCLVKG FYPSDIAVEW | 49) |
| | | ESNGQPENNY KTTPPVLDSD GSFFLYSKLT VDKSRWQQGN | |
| | | VFSCSVMHEA LHNHYTQKSL SLSPG | |
| S | 75 | EDELQVIQPE KSVSVAAGES ATLRCAMTSL IPVGPIMWFR | Wild type |
| | | GAGAGRELIY NQKEGHFPRV TTVSELTKRN NLDFSISISN | SIRPβ1 (SEQ |
| | | ITPADAGTYY CVKFRKGSPD DVEFKSGAGT ELSVRAKPSD | ID NO: 25) |
| | | KTHTCPPCPA PEAAGAPSVF LFPPKPKDTL MISRTPEVTC | fused to IgG1 |
| | | VVVDVSHEDP EVKFNWYVDG VEVHNAKTKP REEQYASTYR | AAA N297A |
| | | VVSVLTVLHQ DWLNGKEYKC KVSNKALPAP IEKTISKAKG | (SEQ ID NO: |
| | | QPREPQVYTL PPSREEMTKN QVSLTCLVKG FYPSDIAVEW | 49) |
| | | ESNGQPENNY KTTPPVLDSD GSFFLYSKLT VDKSRWQQGN | |
| | | VFSCSVMHEA LHNHYTQKSL SLSPG | |
| T | 76 | EEELQVIQPD KSISVAAGES ATLHCTVTSL IPVGPIQWFR | Wild type |
| | | GAGPGRELIY NQKEGHFPRV TTVSDLTKRN NMDFSIRISN | SIRPβ2 (SEQ |
| | | ITPADAGTYY CVKFRKGSPD HVEFKSGAGT ELSVRAKPSD | ID NO: 27) |
| | | KTHTCPPCPA PEAAGAPSVF LFPPKPKDTL MISRTPEVTC | fused to IgG1 |
| | | VVVDVSHEDP EVKFNWYVDG VEVHNAKTKP REEQYASTYR | AAA N297A |
| | | VVSVLTVLHQ DWLNGKEYKC KVSNKALPAP IEKTISKAKG | (SEQ ID NO: |
| | | QPREPQVYTL PPSREEMTKN QVSLTCLVKG FYPSDIAVEW | 49) |
| | | ESNGQPENNY KTTPPVLDSD GSFFLYSKLT VDKSRWQQGN | |
| | | VFSCSVMHEA LHNHYTQKSL SLSPG | |
| U | 77 | EEELQIIQPD KSVLVAAGET ATLRCTITSL FPVGPIQWFR | SIRPα variant |
| | | GAGPGRELIY NQREGPFPRV TTVSDTTKRN NMDFSIRIGA | (SEQ ID NO: |
| | | ITPADAGTYY CVKFRKGSPD DVEFKSGAGT ELSVRAKPSD | 78) fused to |
| | | KTHTCPPCPA PEAAGAPSVF LFPPKPKDTL MISRTPEVTC | IgG1 AAA |
| | | VVVDVSHEDP EVKFNWYVDG VEVHNAKTKP REEQYASTYR | N297A (SEQ |
| | | VVSVLTVLHQ DWLNGKEYKC KVSNKALPAP IEKTISKAKG | ID NO: 49) |
| | | QPREPQVYTL PPSREEMTKN QVSLTCLVKG FYPSDIAVEW | |
| | | ESNGQPENNY KTTPPVLDSD GSFFLYSKLT VDKSRWQQGN | |
| | | VFSCSVMHEA LHNHYTQKSL SLSPG | |
| V | 87 | EEELQIIQPE KLLLVTVGKT ATLHCTITSL FPVGPIQWFR | SIRPγ variant |
| | | GVGPGRVLIY NQKDGPFPRV TTVSDGTKRN NMDFSIRISS | (SEQ ID NO: |
| | | ITPADVGTYY CVKFRKGSPE DVEFKSGPGT EMALGAKPSD | 5) fused to |
| | | KTHTCPPCPA PELLGGPSVF LFPPKPKDTL MISRTPEVTC | wild type |
| | | VVVDVSHEDP EVKFNWYVDG VEVHNAKTKP REEQYNSTYR | IgG1 Fc |
| | | VVSVLTVLHQ DWLNGKEYKC KVSNKALPAP IEKTISKAKG | region (SEQ |
| | | QPREPQVYTL PPSREEMTKN QVSLTCLVKG FYPSDIAVEW | ID NO: 47) |
| | | ESNGQPENNY KTTPPVLDSD GSFFLYSKLT VDKSRWQQGN | |
| | | VFSCSVMHEA LHNHYTQKSL SLSPG | |

Each decoy polypeptide were expressed in Expi293 cells (Invitrogen) using the standard manufacturer's protocol. Expression cultures were typically grown for five days at 37° C. in 8% CO₂. Cell culture supernatants were harvested via centrifugation and were sterile filtered. Proteins were affinity purified utilizing MabSelect Sure LX resin (GE Healthcare) and dialyzed into 1×PBS (Phosphate Buffered Saline, pH 7.4). Purified proteins were separated by SDS-PAGE under either reducing or non-reducing conditions, and detected using Coomassie staining.

Determination of the Binding Affinity (KD) of Decoyvpolyvpeptides for CD47

The binding affinities each decoy polypeptides for CD47 from various species (e.g., human CD47, cynomolgus monkey CD47, and mouse CD47) were determined using indirect capture via biotinylated Protein A (via NLC chip). All experiments were performed at 25° C. using a Surface Plasmon Resonance (SPR)-based ProteOn XPR36 biosensor (BioRad, Inc., Hercules, Calif.). The running buffer was PBS at pH 7.4 with 0.01% Tween-20 (PBST+). All analytes were used at their nominal concentrations as determined by A₂₈₀ absorbance and using their molar calculated extinction coefficients. CD47 analytes were injected in a “one-shot” kinetic mode as described elsewhere (See, e.g., Bravman et al., (2006) Anal. Biochem. 358:281-288).

As a first step, 15 μg/mL biotinylated protein A (Thermofisher) was injected at 30 μL/min for 120 seconds over the NLC chip to obtain an immobilization response of about 1000-1200 RUs. Next, decoy polypeptides (about 100-160 nM) were injected for 80 seconds at 30 μL/min. The CD47 analytes (from human, cynomolgus monkey, and mouse) were subsequently injected in a “one-shot” kinetic mode at nominal concentrations of 100 nM, 33 nM, 11 nM, 3.7 nM, 1.2 nM, and 0 nM. Association times were monitored for 60 seconds at 25 μL/min, and dissociation times were monitored for 500 seconds. The surfaces were regenerated with a 2:1 v/v blend of Pierce IgG elution buffer/4M NaCl. Biosensor data were double-referenced by subtracting the interspot data (containing no immobilized protein) from the reaction spot data (immobilized protein) and then subtracting the response of a buffer “blank” analyte injection from that of an analyte injection. Double-referenced data were fit globally to a simple Langmuir model and the K_{D} value was calculated from the ratio of the apparent kinetic rate constants (K_{D}=k_{d}/kₐ).

Identification of Decoy Polypeptides that Block Binding of SIRPα to CD47

To determine whether high affinity decoy polypeptides block binding of SIRPα to CD47, SPR screens were carried out. Decoy polypeptides were captured to surface-immobilized Protein A prepared as described above. A high affinity SIRPα d1 domain variant (SEQ ID NO:78) engineered to bind CD47 with high nM affinity was used for the screen rather than a wild type SIRPα, as wild type SIRPα has low μM binding affinity to CD47. (Low RM binding affinity does not allow for stable complex interaction to assess sandwich formation in SPR assays.) First, approximately 100 nM of purified decoy polypeptide was injected for 80s at 30 μl/min and captured over the immobilized protein A followed by a brief buffer flow of 1 min at 100 μL/min. Next, 100 nM of human CD47 (QLLFNKTKSV EFTFSNDTVV IPCFVTNMEA QNTTEVYVKW KFKGRDIYTF DGALNKSTVP TDFSSAKIEV SQLLKGDASL KMDKSDAVSH TGNYTCEVTE LTREGETIIE LKYRVVS (SEQ ID NO: 80)) premixed with the high affinity SIRPα variant at different concentrations of 0, 20, 55, 500, or 1500 nM was injected separately for one minute at 100 μL/min with a dissociation time of 10 minutes.

### Results

Expression and Purification of Decoy Polypeptides

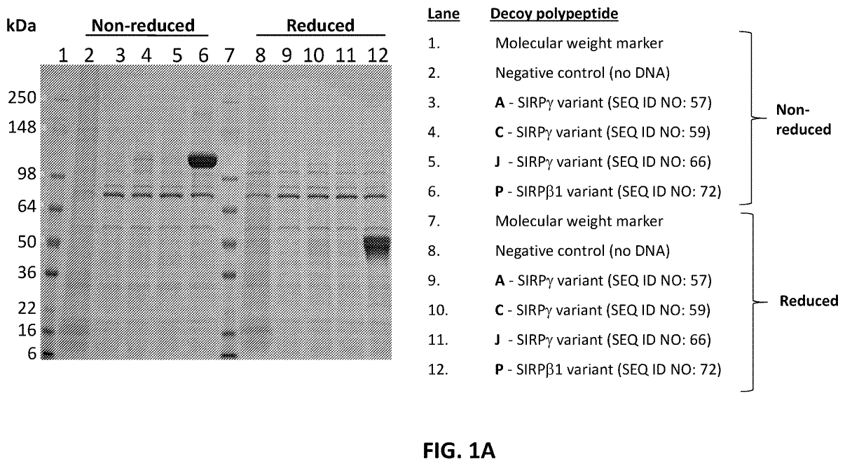

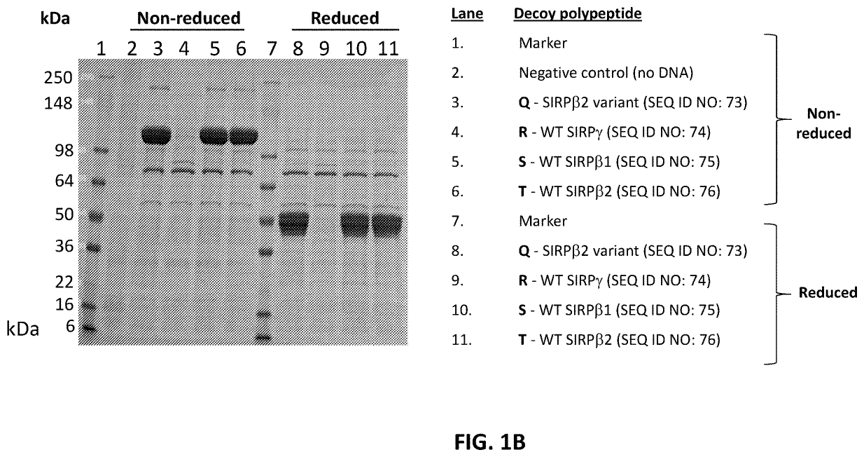

Non-reducing SDS-PAGE analysis of purified decoy polypeptides revealed good expression of decoy polypeptide P (SEQ ID NO: 72), which comprises a SIRPβ1 D1 domain variant (see lane 6 of FIG. 1A), and decoy polypeptide S (SEQ ID NO: 75), which comprises a wild type SIRPβ1 D1 domain (See lane 5 of FIG. 1B). Similarly, good expression was observed for decoy polypeptide T (SEQ ID NO: 76), which comprises a wild type SIRPβ2 D1 domain (See lane 6 of FIG. 1B) and for decoy polypeptide Q (SEQ ID NO: 73), which comprises a variant SIRPβ2 D1 domain (See lane 3 of FIG. 1B).

In contrast, decoy polypeptide R (SEQ ID NO: 74), which comprises a wild type SIRPγ D1 domain, was expressed at a low level, as no visible overexpression was observed in the SDS-PAGE analysis (see lane 4 in FIG. 1B). Similarly, decoy polypeptides A (SEQ ID NO: 57), C (SEQ ID NO: 59), and J (SEQ ID NO: 66), which each comprise a different SIRPγ D1 domain variant, were expressed at low levels (see lanes 3-5 of FIG. 1A).

To determine whether decoy polypeptides were present as dimers linked by disulfide bonds, the SDS-PAGE analysis was also carried out under reducing conditions that disrupt disulfide bonds in proteins. As shown in FIG. 1A, the high molecular weight band observed between 98 kDa and 148 kDa in non-reduced SDS-PAGE for the decoy polypeptide P (see lane 6 in FIG. 1A) disappeared in the reduced SDS-PAGE analysis, and a lower molecular weight band (˜50 kDa) appeared (see lane 12 in FIG. 1A). These results indicated that decoy polypeptide P was expressed as a dimer. Similarly, as shown in FIG. 1B, the high molecular weight bands observed between 98 kDa and 148 kDa in non-reduced SDS-PAGE disappeared in the reduced SDS-PAGE analysis, and lower molecular weight bands (˜50 kDa) appeared for decoy polypeptides Q, R, S, and T, indicating that decoy polypeptides Q, R, S, and T were also present as dimers. It is likely that decoy polypeptide dimers are linked by disulfide bonds, possibly through the Fc domains. Binding kinetics of SIRPγ variants to human CD47

The affinities (K_{D}) of decoy polypeptides comprising SIRPβγ D1 domain variants or a wild-type SIRPγ D1 domain for human CD47 were determined by SPR. As shown in Table 3, several decoy polypeptides comprising SIRPγ D1 domain variants had improved affinity for hCD47 as compared to the decoy polypeptide comprising a wild type SIRPγ D1 domain. Decoy polypeptides B (SEQ ID NO: 58), C (SEQ ID NO: 59), D (SEQ ID NO: 60), F (SEQ ID NO: 62), G (SEQ ID NO: 63), H (SEQ ID NO: 64), J (SEQ ID NO: 66), and L (SEQ ID NO: 68), which each comprise a different SIRPγ D1 domain variant, bound to human CD47 with affinities that were between with between 545- to 9012-fold higher than the affinity of decoy polypeptide R (SEQ ID NO: 74) for human CD47. (As noted in Table 2 above and in Table 3 below, decoy polypeptide R comprises a wild type SIRPγ D1 domain.)

TABLE 3
Binding kinetics of decoy polypeptides comprising SIRPγ
variants or wild type SIRPγ to human CD47.

| | | | Fold | |
|---|---|---|---|---|
| | | | improvement | |
| Decoy | SEQ | K_{D} (M) | in KD vs wild | |
| polypeptide | ID NO | for hCD47* | type‡ | Description |
| A | 57 | 7.48E−08 | | SIRPγ variant |
| | | | | (SEQ ID NO: 3) |
| B | 58 | 3.20E−10 | 6844 | SIRPγ variant |
| | | | | (SEQ ID NO: 4) |
| C | 59 | 2.43E−10 | 9012 | SIRPγ variant |
| | | | | (SEQ ID NO: 5) |
| D | 60 | 1.80E−09 | 1217 | SIRPγ variant |
| | | | | (SEQ ID NO: 6) |
| E | 61 | No binding | | SIRPγ variant |
| | | | | (SEQ ID NO: 7) |
| F | 62 | 8.81E−10 | 2486 | SIRPγ variant |
| | | | | (SEQ ID NO: 8) |
| G | 63 | 1.56E−09 | 1404 | SIRPγ variant |
| | | | | (SEQ ID NO: 10) |
| H | 64 | 4.83E−10 | 4534 | SIRPγ variant |
| | | | | (SEQ ID NO: 11) |
| I | 65 | No binding | | SIRPγ variant |
| | | | | (SEQ ID NO: 13) |
| J | 66 | 2.75E−10 | 7964 | SIRPγ variant |
| | | | | (SEQ ID NO: 17) |
| K | 67 | No binding | | SIRPγ variant |
| | | | | (SEQ ID NO: 18) |
| L | 68 | 4.02E−09 | 545 | SIRPγ variant |
| | | | | (SEQ ID NO: 19) |
| M | 69 | No binding | | SIRPγ variant |
| | | | | (SEQ ID NO: 21) |
| N | 70 | No binding | | SIRPγ variant |
| | | | | (SEQ ID NO: 22) |
| O | 71 | No binding | | SIRPγ variant |
| | | | | (SEQ ID NO: 42) |
| R | 74 | 2.19E−06 | 1 | Wild type SIRPγ |
| | | | | (SEQ ID NO: 1) |

*The amino acid sequence of hCD47 is set forth in SEQ ID NO: 80.
‡Decoy polypeptide R comprises a wild type SIRPγ D1 domain

Binding Kinetics of SIRPβ Variants to Human CD47

The affinities (K_{D}) of decoy polypeptides comprising a SIRPβ1 d1 domain variant, a SIRPβ32 D1 domain variant, a wild type SIRPβ1 d1 or a wild type SIRPβ2 d1 domain for human CD47 were determined by SPR. As shown in Table 4, decoy polypeptides S (SEQ ID NO: 75), which comprises a wild type SIRPβ1 d1 domain and decoy polypeptide T (SEQ ID NO: 76), which comprises a wild type SIRPβ32 D1 domain, did not bind to human CD47. Surprisingly, decoy polypeptides P (SEQ ID NO: 72), which comprises a SIRPβ1 d1 domain variant, and Q (SEQ ID NO: 73 which comprises a SIRPβ2 d1 domain variant, bound to human CD47 with K_{D} values in the range of 0.21 nM to 0.35 nM.

TABLE 4
Binding kinetics of decoy polypeptides comprising a wild type
SIRPβ1 d1 domain, a wild type SIRPβ2 d1 domain, a IRPβ1 d1
domain variant, or a SIRPβ2 d1 domain variant to human CD47.

| Decoy | SEQ | K_{D} (M) | |
|---|---|---|---|
| polypeptide | ID NO | for hCD47* | Description |
| P | 72 | 3.50E−10 | SIRPβ1 variant |
| | | | (SEQ ID NO: 26) |
| S | 75 | No binding | Wild type SIRPβ1 |
| | | | (SEQ ID NO: 25) |
| Q | 73 | 2.13E−10 | SIRPβ2 variant |
| | | | (SEQ ID NO: 28) |
| T | 76 | No binding | Wild type SIRPβ2 |
| | | | (SEQ ID NO: 27) |

*The amino acid sequence of hCD47 is set forth in SEQ ID NO: 80.

### Binding Kinetics of SIRPγ and SIRPβ Variants to Human, Cynomolgus Monkey, and Mouse CD47

Next, the affinities (K_{D}) of decoy polypeptides comprising a wild SIRPβ1, SIRPβ2, or SIRPγ D1 domains for human, cynomolgus monkey, and mouse CD47 were determined by SPR.

As shown in Table 4, decoy polypeptide R (SEQ ID NO: 74), which comprises a wild type human SIRPγ D1 domain, did not bind to mouse CD47. In contrast, decoy polypeptides C (SEQ ID NO: 59) and J (SEQ ID NO: 66), which each comprise a different SIRPγ D1 domain variant, bound with high affinities to mouse CD47, with K_{D} values of 0.9 nM and 1.3 nM, respectively (see Table 4). In addition, decoy polypeptides C and J also bound with high affinities to cynomolgus monkey CD47, with K_{D} values of 2.74E-10 and 3.30E-10, respectively.

Decoy polypeptides S (SEQ ID NO: 75), which comprises a wild type human SIRPβ D1 domain, and decoy polypeptide T (SEQ ID NO: 76), which comprises a wild type human SIRPβ D1 domain, exhibited no binding to human CD47, cynomolgus monkey CD47, or mouse CD47. In contrast, decoy polypeptide P (SEQ ID NO: 72), which comprises a SIRPβ D1 domain variant, and decoy polypeptide Q (SEQ ID NO: 73), which comprises a SIRPβ2 D1 domain variant, exhibited some binding to mouse CD47 and bound with high affinities to cynomolgus monkey CD47. As shown in the last column of Table 4, decoy polypeptides C, J, P, and Q each blocked the binding of SIRPα to CD47.

Decoy polypeptide U (SEQ ID NO: 77), which comprises a SIRPα D1 domain variant, was used as a positive control. Decoy polypeptide U bound with high affinity to human (K_{D}=0.19 nM), cynomolgus monkey (K_{D}=0.22 nM), and mouse CD47 (K_{D}=7.8 nM).

TABLE 4
Binding kinetics of decoy polypeptides to human, cynomolgus monkey, and mouse CD47.

| | | | | | | Blocking |
|---|---|---|---|---|---|---|
| Decoy | | SEQ | Binding to CD47 (K_{D} = M) | | | CD47:SIRPα |
| polypeptide | Description | ID NO: | Human | Cyno | Mouse | interaction |
| C | SIRPγ variant | 59 | 2.43E−10 | 2.74E−10 | 9.05E−10 | Yes |
| | (SEQ ID NO: 5) | | | | | |
| J | SIRPγ variant | 66 | 3.98E−10 | 3.30E−10 | 1.30E−09 | Yes |
| | (SEQ ID NO: 17) | | | | | |
| R | Wild type SIRPγ | 74 | 2.19E−06 | No binding | No binding | Not tested |
| | (SEQ ID NO: 1) | | | | | |
| P | SIRPβ1 variant | 72 | 2.88E−10 | 3.34E−10 | 1.46E−07 | Yes |
| | (SEQ ID NO: 26) | | | | | |
| S | Wild type SIRPβ1 | 75 | No binding | No binding | No binding | N.A. |
| | (SEQ ID NO: 25) | | | | | |
| Q | SI RPβ2 variant | 73 | 7.85E−11 | 1.06E−10 | 2.41E−08 | Yes |
| | (SEQ ID NO: 28) | | | | | |
| T | Wild type SIRPβ2 | 76 | No binding | No binding | No binding | N.A |
| | (SEQ ID NO: 27) | | | | | |
| U | SIRPα variant | 77 | 1.93E−10 | 2.24E−10 | 7.82E−09 | Yes |
| | (SEQ ID NO: 78) | | | | | |

### Example 2: Sequence Analysis of SIRPγD1 Domain Variants, a SIRPα D1 Domain Variant, a SIRPβ1 D1 Domain Variant, and a SIRPβ2 D1 Domain Variant

In the following example, the amino acid sequences of SIRPγ, SIRPα, SIRPβ1, and SIRPβ2 D1 domain variants were analyzed to identify residues that were important for improved binding to CD47.

Wild type human SIRPβ1 and wild type human SIRPβ2 do not bind human CD47 (e.g., See, Tables 3-4), whereas wild type human SIRPγ binds with low μM affinity to human CD47. Wild type human SIRPα binds to human CD47 with 10 fold higher affinity than wild type human SIRPγ.

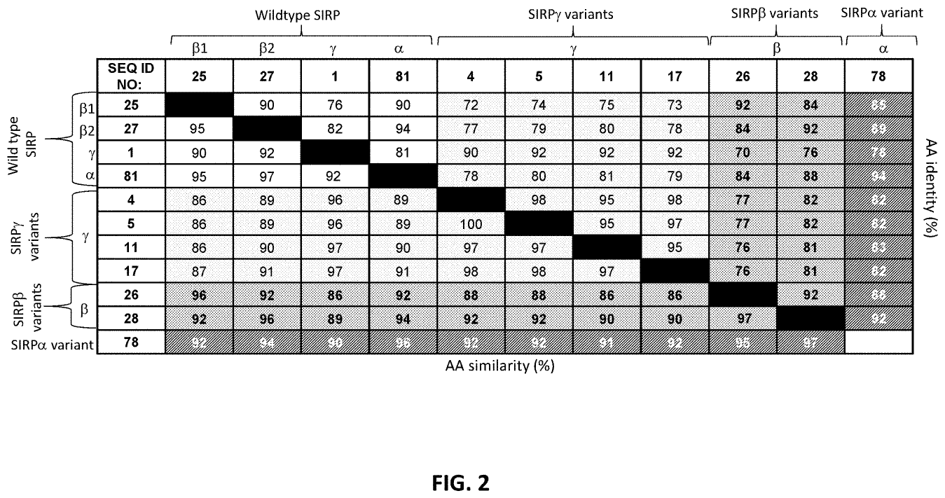

Notwithstanding the differences in binding affinities for CD47 described above, the wild type SIRPα D1 domain (SEQ ID NO: 81) has higher sequence identity to the wild type D1 domains of SIRPβ1 (SEQ ID NO: 25) and SIRPβ2 (SEQ ID NO: 27) than to the wild type D1 domain of SIRPγ (SEQ ID NO: 1). As shown in FIG. 2, wild type SIRPα D1 domain (SEQ ID NO: 81) had 90% and 94% sequence identity to wild type D1 domains of SIRPβ1 (SEQ ID NO: 25) and SIRPβ2 (SEQ ID NO: 27), respectively. In contrast, wild type SIRPα D1 domain had 81% sequence identity to wild type SIRPγ D1 domain (SEQ ID NO: 1). Similarly, wild type SIRPα D1 domain had 95% and 97% sequence similarity to wild type D1 domains of SIRPβ1 and SIRPβ2, respectively, while it had 92% sequence similarity to wild type SIRPγ.

As shown in FIG. 2, decoy polypeptides comprising SIRPγ, SIRPβ, SIRPβ2, or SIRPα D1 domain variants that exhibited improved affinities to CD47 (nM-pM) relative to wild type displayed varied percentage sequence identities and similarities among each other. Sequence similarity was defined as the percentage of identical and similar amino acids between each sequence pair among all un-gapped positions. Sequence identity was defined as the percentage of identical residues between each sequence pair among all un-gapped positions.

The SIRPβ D1 domain variant and the SIRPγ D1 domain variants shared between 76% and 82% amino acid sequence identity. Similarly, the sequences of the SIRPα domain variant and the SIRPγ D1 domain variants were approximately 82% identical. The SIRPα D1 domain variant shared 92% amino acid sequence identity with the SIRPβ D1 domain variant and 88% amino acid sequence identity the SIRPβ2 D1 domain variant.

Identification and Structural Modeling of Sequence Differences Between Wild Type and High Affinity Variant SIRPβ1 D1 Domains

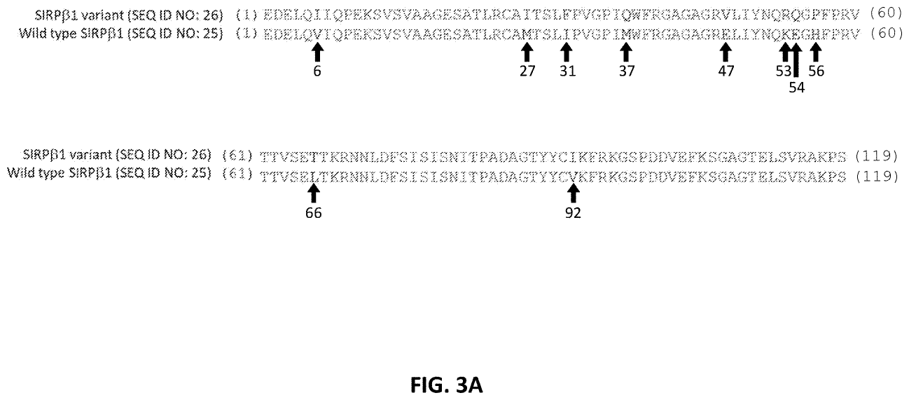

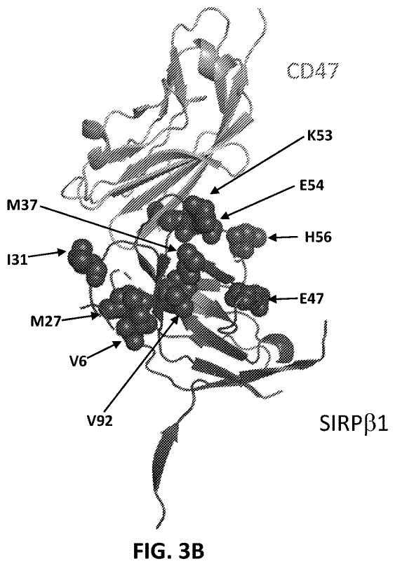

As shown in FIG. 3A, sequence alignments of the wild type SIRPβ1 D1 domain (SEQ ID NO: 25) and the SIRPβ1 D1 domain variant (SEQ ID NO: 26) revealed ten amino acid differences: V6I, M27I, I31F, M37Q, E47V, K53R, E54Q, H56P, L66T, and V92I. The ten residues that differ between the wild type SIRPβ1 D1 domain and the SIRPβ1 D1 domain variant are highlighted as spheres in the structural model shown in FIG. 3B. FIG. 3B shows a wild type human SIRPβ1 D1 domain X-ray crystal structure (PDB: 2JJU) superimposed onto a crystal structure of the SIRPα D1 domain bound to CD47 (PDB: 2JJS).

Identification and Structural Modeling of Sequence Differences Between Wild Type and High Affinity Variant SIRPβ2 D1 Domains

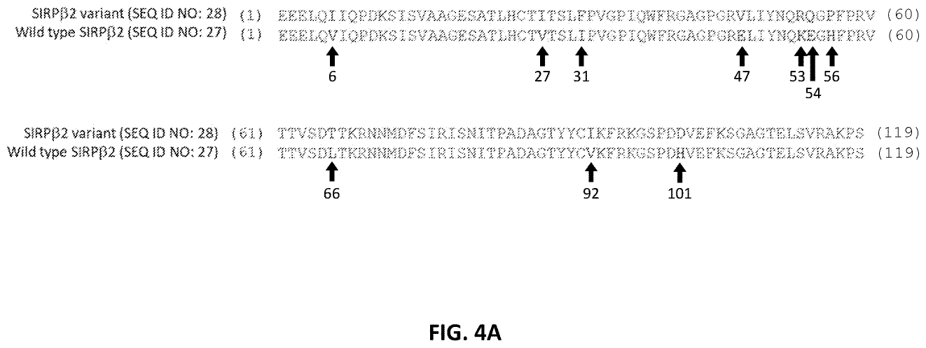

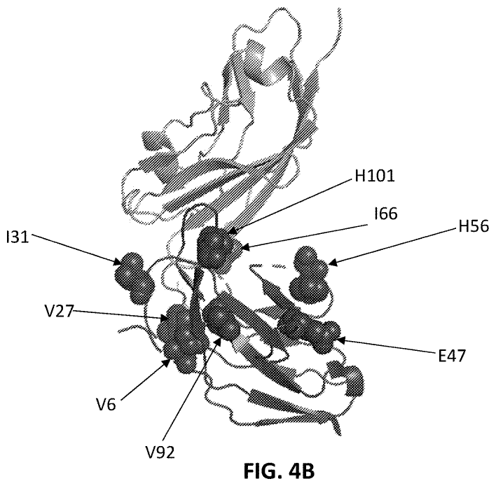

As shown in FIG. 4A, sequence alignments of the wild type SIRPβ2 D1 domain (SEQ ID NO: 27) and a SIRPβ2 D1 domain variant (SEQ ID NO: 28) also revealed ten amino acid differences: V6I, V27I, I31F, E47V, K53R, E54Q, H56P, L66T, V92I, and H101D. The ten residues that differ between wild type and high affinity variant SIRPβ2 D1 domains are highlighted as spheres in the structural model shown in FIG. 4B. FIG. 4B shows a wild type SIRPβ2 D1 domain X-ray crystal structure (PDB: 2JJV) superimposed onto a crystal structure of the SIRPα D1 domain bound to CD47 (PDB: 2JJS). (Residues K53 and E54 are not visible in FIG. 4B.)

Identification and Structural Modeling of Sequence Differences Between Wild Type and Variant SIRPγD1 Domains

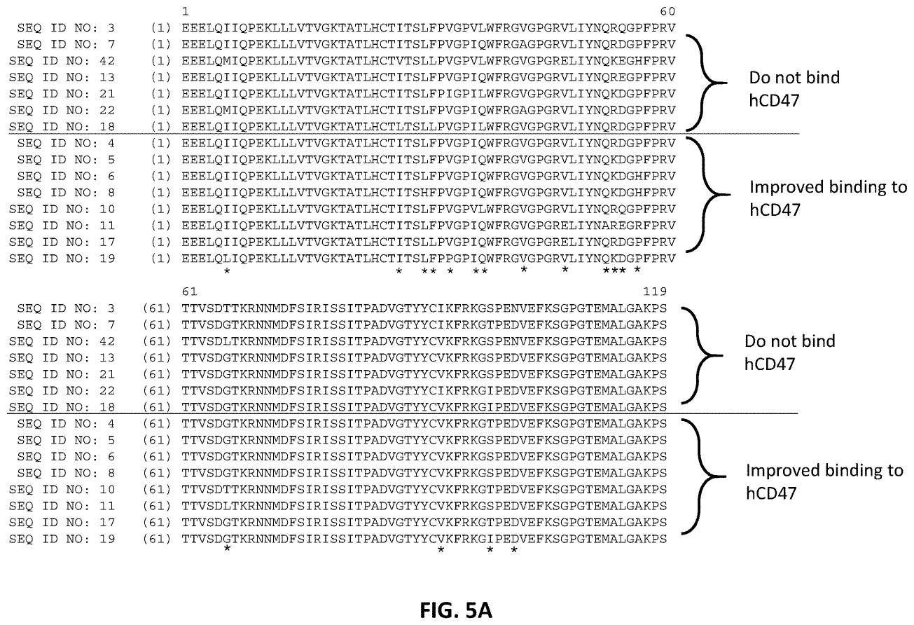

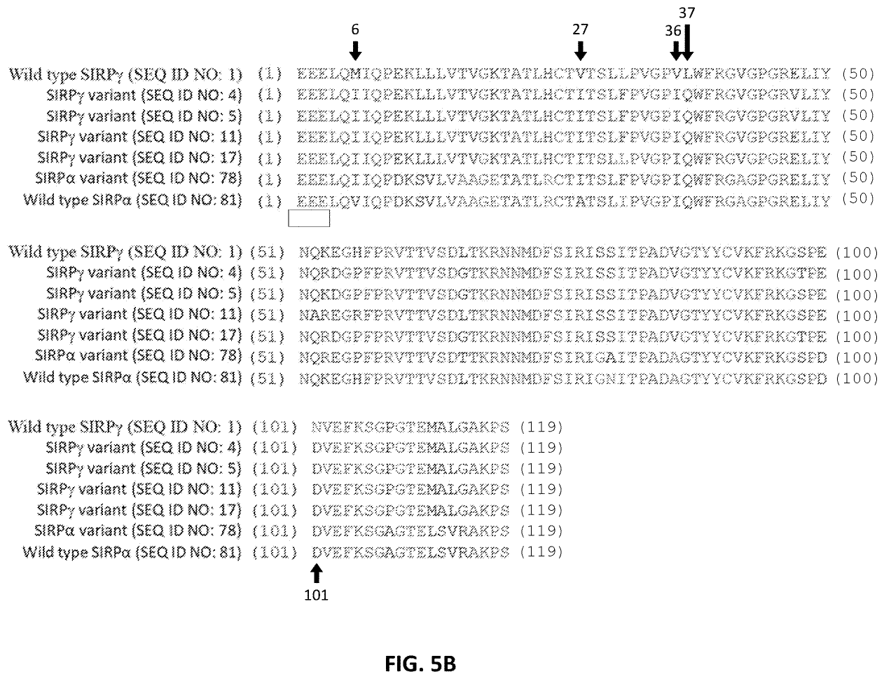

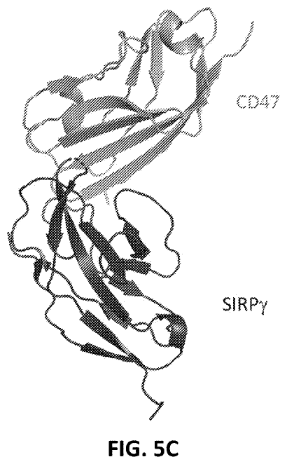

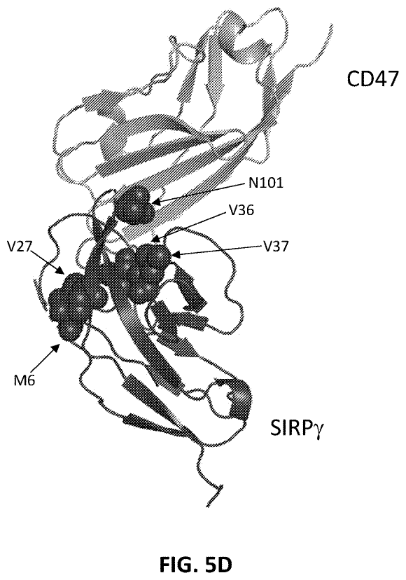

As shown in FIG. 5A, alignment of the sequences of the SIRPγ D1 domain variants described in Example 1 revealed no clear amino acid requirements for improved binding to human CD47. As shown in FIG. 5B, sequence comparisons of the wild type SIRPγ D1 domain to the four variant SIRPγ D1 domains that demonstrated highest affinities for CD47 in Table 3 (SEQ ID NOs: 4, 5, 11, 17, with affinities in the range of 0.2 nM to 0.5 nM) revealed that each of these variants comprise the same substitutions at five amino acid positions: M6I, V27I, V36I, L37Q, and N101D. The SIRPα D1 domain variant of SEQ ID NO: 78 comprises substitutions at two of these amino acid positions, i.e., V6I and A27I, whereas the amino acids at positions 36, 37, and 101 are unsubstituted. The amino acids at the unsubstituted positions in SEQ ID NO: 78 are 136, Q37, and D101.

A crystal structure of the SIRPγ D1 domain bound to CD47 is shown in FIG. 5C (PDB: 2JJW). In FIG. 5D, the five amino acid residues that were mutated in all four SIRPγ D1 domain variants with the highest affinities for human CD47 are highlighted as spheres.

Alignment of SIRPα, SIRP9l, SIRP92, and SIRPγD1 Domains

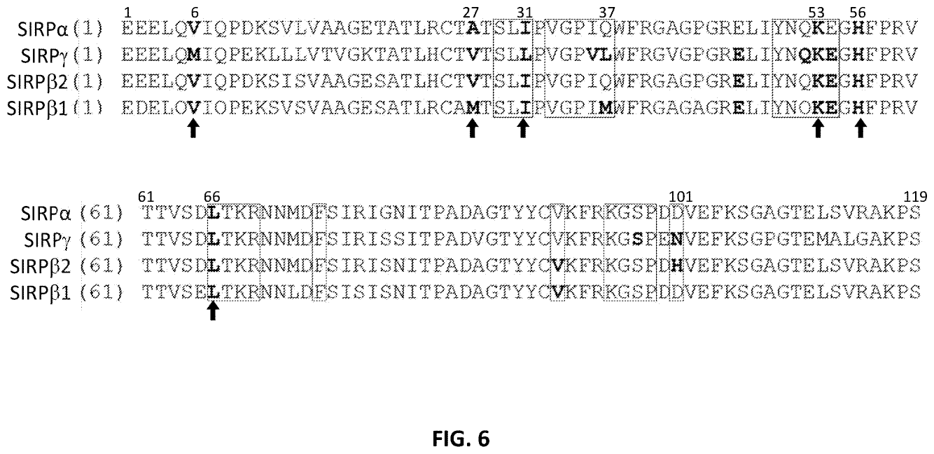

The sequences of wild type SIRPα, SIRPβ1, SIRPβ2, and SIRPγ D1 domains were aligned to identify amino acid residue differences and to determine the amino acid positions which, when substituted, improve binding to CD47. As shown in FIG. 6, the residues that were mutated in the SIRPα, SIRPβ31, SIRPβ2, and SIRPγ D1 domain variants that demonstrated improved binding to CD47 relative to wild type are bolded. Six amino acid residues (i.e., positions 6, 27, 31, 53, 56, and 66) were mutated in each of the variants (indicated with arrows). Of these six residues, positions 27, 31, 53, 56, and 66) are in or near regions that were previously characterized to be binding sites for CD47 on SIRPα (boxed regions).

### Example 3: Decoy Polypeptides Comprising Variant SIRPα, SIRPβ1, SIRPβ2, and SIRPγD1 Domains Enhance Phagocytosis of Tumor Cells by Macrophages

The following example demonstrates that decoy polypeptides that bind to human CD47 with high affinity (see Example 1) enhance in vitro phagocytosis of tumor cells by macrophages in combination with cetuximab.

### Materials and Methods

In Vitro Phagocytosis Assays

DLD-1 cells were detached from culture plates by washing twice with 20 ml PBS and incubating in 10 ml TrypLE Select (Gibco) for 10 minutes at 37° C. Cells were centrifuged, washed in PBS, and resuspended in medium. Cells were labeled with the Celltrace CFSE Cell Proliferation kit (Thermo Fisher) according to the manufacturer's instructions and resuspended in IMDM. Macrophages were detached from culture plates by washing twice with 20 ml PBS and incubating in 10 ml TrypLE Select for 20 minutes at 37° C. Cells were removed with a cell scraper (Corning), washed in PBS, and resuspended in IMDM.

Phagocytosis assays were assembled in ultra-low attachment U-bottom 96 well plates (Corning) containing 100,000 DLD-1 cells, 50,000 macrophages, five-fold serial dilutions of decoy polypeptides (from 100 nM to 6.4 pM, or 1 μM to 64 pM), and cetuximab at 0.01 μg/ml or control antibody of the same isotype. Plates were incubated two hours at 37° C. in a humidified incubator with 5 percent carbon dioxide. Cells were pelleted by centrifugation for five minutes at 400×g and washed in 250 μl FACS buffer. Macrophages were stained on ice for 15 minutes in 50 μl FACS buffer containing 10¹ human FcR Blocking Reagent (Miltenyi Biotec), 0.5 μl anti-CD33 BV421 (Biolegend), and 0.5 μl anti-CD206 APC-Cy7 (Biolegend). Cells were washed first in 200 μl FACS buffer, and then in 250 μl PBS. Cells were then stained on ice for 30 minutes in 50 μl Fixable Viability Dye eFluor 506 (eBioscience) diluted 1:1000 in PBS. Cells were washed twice in 250 μl FACS buffer and fixed overnight in 0.5% paraformaldehyde. Cells were analyzed on a FACS Canto II (BD Biosciences), with subsequent data analysis by Flowjo 10.7 (Treestar). Dead cells were excluded by gating on the e506-negative population. Macrophages that had phagocytosed tumor cells were identified as cells positive for CD33, CD206, and CFSE.

### Results

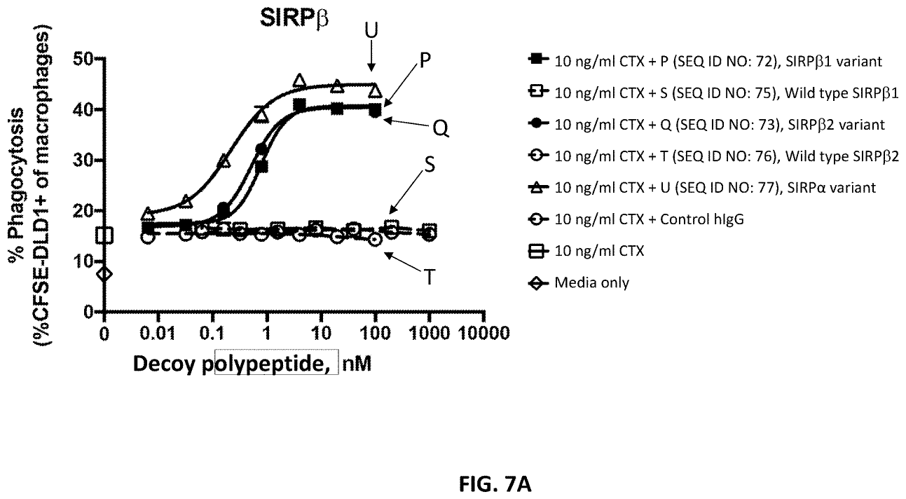

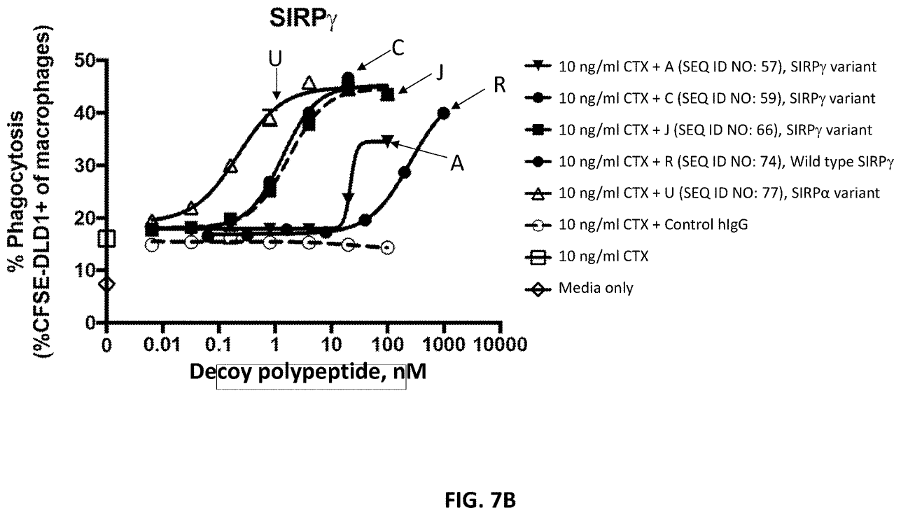

As shown in FIG. 7A, phagocytosis of CFSE-labeled DLD-1 tumor cells by human monocyte-derived macrophages in the presence of cetuximab (CTX; 10 ng/ml), an EGFR inhibitor, was not enhanced by decoy polypeptide S, which comprises a wild type SIRPβ1 D1 domain, or decoy polypeptide T, which comprises a wild type SIRPβ2 D1 domain. In contrast, decoy polypeptides P, Q, and U, each enhanced phagocytosis of DLD-1 tumor cells by macrophages in combination with cetuximab.

Decoy polypeptide R, which comprises a wild type SIRPγ D1 domain, potentiated phagocytosis of DLD-1 tumor cells by macrophages poorly in combination with cetuximab (FIG. 7B). In contrast, decoy polypeptides C and J, which each comprise a different SIRPγ D1 domain variant, strongly enhanced phagocytosis of DLD-1 tumor cells by macrophages in combination with cetuximab, as did decoy polypeptide U.

Overall, the results presented in this example show that decoy polypeptides containing variant SIRPα, SIRPβ1, SIRPβ2, and SIRPγ D1 domains with improved binding to CD47 enhance the phagocytosis of tumor cells by macrophages when combined with an anti-tumor antigen antibody, such as cetuximab.

### Example 4: Administration of a Decoy Polypeptide Comprising an Fe Variant does not Affect Hematological Parameters

A first group of 12 female CD-1 mice were administered intravenously with 10 mg/kg decoy polypeptide V, which comprises the SIRPγ d1 domain variant of SEQ ID NO: 5 and the wild type human IgG1 Fc region of SEQ ID NO: 47, and a second group of 6 female CD-1 mice were administered intravenously with 10 mg/kg decoy polypeptide C, which comprises the SIRPγ d1 domain variant of SEQ ID NO: 5 and the Fc inactive hIgG1 of SEQ ID NO: 49. See Table 2. Animals were observed for a minimum of an hour following dosing, then minimally once daily, increasing if any clinical abnormalities were observed. For complete blood counts (CBC) analysis, blood was collected via tail vein into K2EDTA microcapillary tubes (Heska) 8 hours prior to administration of decoy polypeptide (i.e., “−8”), 3 days following administration, and 8 days following administration. Hematologic parameters were evaluated using a HeskaView analyzer

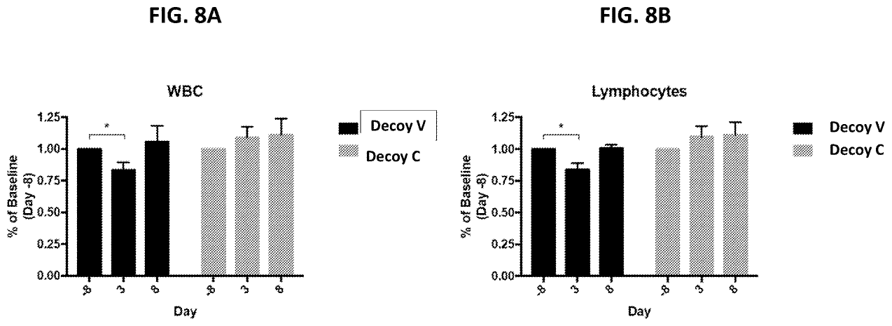

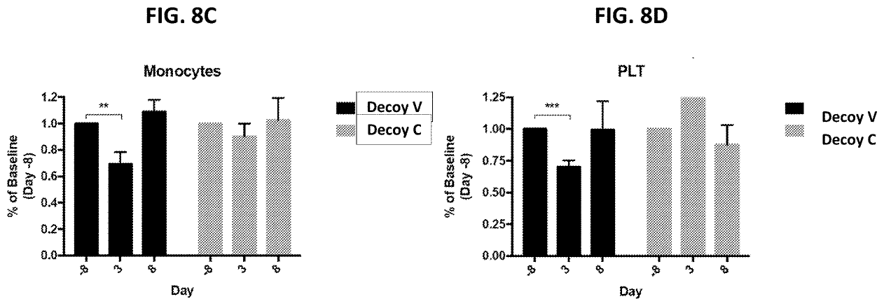

Immediately following administration, mice dosed with decoy polypeptide V showed clinical signs of stress by demonstrating a sudden lack of movement, but recovered 30-60 minutes post dosing. As shown in FIGS. 8A-8D, administration of decoy polypeptide C, which lacks Fc effector function, had little effect on hematology parameters. In mice given 10 mg/kg decoy polypeptide C, levels of platelets (PLT) (FIG. 8D), and white blood cells (WBC: lymphocytes, monocytes, and granulocytes) (FIG. 8A) at Days 3 and 8 following administration were similar to the levels at pre-dose baseline (i.e., 8 hours prior to administration, or “−8”). By contrast, administration of decoy polypeptide V resulted in a decrease in hematological parameters. Administration of 10 mg/kg decoy polypeptide V to mice resulted in 30% platelet reduction (FIG. 8D), 17% WBC reduction (FIG. 8A), 17% lymphocyte reduction (FIG. 8B), and 30% monocyte reduction (FIG. 8C) within three days post dosing as compared to levels at pre-dose baseline (i.e., 8 hours prior to administration, or “−8”) (unpaired t-test, *p<0.05 and **p<0.005). By day eight, all hematological parameters that were tested returned to baseline in mice given decoy polypeptide V. These results demonstrate that administration of an exemplary decoy polypeptide comprising a SIRPγ variant capable of blocking the interaction of SIRPα and CD47 and an Fc variant with reduced effector function (see Table 4) does not result in adverse effects on normal blood cells.

1. A decoy polypeptide comprising:
(a) a SIRPγ variant; and
(b) a human Fc variant comprising at least one amino acid substitution that reduces effector function compared to a wild type human Fc,
wherein the SIRPγ variant comprises at least one amino acid substitution relative to a wild type SIRPγ, which substitution increases the affinity of the SIRPγ variant for CD47 as compared to the affinity of the wild type SIRPγ for CD47, and wherein the SIRPγ variant lacks a transmembrane domain.

2. The decoy polypeptide of claim 1, wherein the at least one amino acid substitution is within a d1 domain of the SIRPγ variant.

3. The decoy polypeptide of claim 1 or claim 2, wherein the amino acid sequence of the d1 domain of the SIRPγ variant is at least 90% identical to a sequence of a wild type SIRPγ d1 domain set forth in
(SEQ ID NO: 1)
EEELQMIQPEKLLLVTVGKTATLHCTVTSLLPVGPVLWFRGVGPGRELIYN
QKEGHFPRVTTVSDLTKRN NMDFSIRISSITPADVGTYY CVKFRKGSPE
NVEFKSGPGTEMALGAKPS.

4. The decoy polypeptide of any one of claims 1 to 3, wherein the SIRPγ variant comprises one or more amino acid substitutions at M6, V27, L30, L31, V33, V36, L37, V42, E47, Q52, K53, E54, H56, L66, T67, V92, S98 or N101, wherein the amino acid positions are relative to the wild-type human SIRPγ d1 domain sequence set forth in SEQ ID NO: 1.

5. The decoy polypeptide of claim 4, wherein the SIRPγ variant comprises the M6 substitution, and wherein the substitution is M6I, M6L or M6F.

6. The decoy polypeptide of claim 4 or 5, wherein the SIRPγ variant comprises the V27 substitution, and wherein the substitution is V27F, V27I or V27L.

7. The decoy polypeptide of any one of claims 4 to 6, wherein the SIRPγ variant comprises the L30 substitution, and wherein the substitution is L30I, L30V, L30H, L30N or L30D.

8. The decoy polypeptide of any one of claims 4 to 7, wherein the SIRPγ variant comprises the L31 substitution, and wherein the substitution is L31F, L31I, L31V, L31T, or L31S.

9. The decoy polypeptide of any one of claims 4 to 8, wherein the SIRPγ variant comprises the V33 substitution, and wherein the substitution is V33I, V33L, V33P, V33T, or V33A.

10. The decoy polypeptide of any one of claims 4 to 9, wherein the SIRPγ variant comprises the V36 substitution, and wherein the substitution is V36I.

11. The decoy polypeptide of any one of claims 4 to 10, wherein the SIRPγ variant comprises the L37 substitution, and wherein the substitution is L37Q.

12. The decoy polypeptide of any one of claims 4 to 11, wherein the SIRPγ variant comprises the V42 substitution, and wherein the substitution is V42A.

13. The decoy polypeptide of any one of claims 4 to 12, wherein the SIRPγ variant comprises the E47 substitution, and wherein the substitution is E47V.

14. The decoy polypeptide of any one of claims 4 to 13, wherein the SIRPγ variant comprises the Q52 substitution, and wherein the substitution is Q52P, Q52L, Q52V, Q52A or Q52E.

15. The decoy polypeptide of any one of claims 4 to 14, wherein the SIRPγ variant comprises the K53 substitution, and wherein the substitution is K53R.

16. The decoy polypeptide of any one of claims 4 to 15, wherein the SIRPγ variant comprises E54 substitution, and wherein the substitution is E54D, E54K, E54N, E54Q, or E54H.

17. The decoy polypeptide of any one of claims 4 to 16, wherein the SIRPγ variant comprises the H56 substitution, and wherein the substitution is H56P or H56R.

18. The decoy polypeptide of any one of claims 4 to 17, wherein the SIRPγ variant comprises the L66 substitution, and wherein the substitution is L66I, L66V, L66P, L66T, L66A, L66R, L66S or L66G.

19. The decoy polypeptide of any one of claims 4 to 18, wherein the SIRPγ variant comprises the T67 substitution, and wherein the substitution is T67I, T67N, T67F, T67S, T67Y, T67V, T67A or T67D.

20. The decoy polypeptide of any one of claims 4 to 19, wherein the SIRPγ variant comprises the V92 substitution, and wherein the substitution is V92I.

21. The decoy polypeptide of any one of claims 4 to 20, wherein the SIRPγ variant comprises the S98 substitution, and wherein the substitution is S98R, S98N, S98K, S98T, S981 or S98M.

22. The decoy polypeptide of any one of claims 4 to 21, wherein the SIRPγ variant comprises the N101 substitution, and wherein the substitution is N101K, N101D, N101E, N101H or N101Q.

23. The decoy polypeptide of any one of claims 1 to 4, wherein the SIRPγ variant comprises an amino acid sequence set forth in EEELQX₁IQPEKLLLVTVGKTATLHCTX₂TSX₃X₄PX₅GPX₆X₇WFRGX₈GPGRX₉LIYNX₁₀X₁₁X₁₂G X₁₃FPRVTTVSDX₁₄X₁₅KRNNMDFSIRISSITPADVGTYYCX₁₆KFRKGX₁₇PEX₁₈VEFKSGPGTEM ALGAKPS (SEQ ID NO: 2), wherein X₁ is M, I, L or F; X₂ is F, I, L or V; X₃ is L, I, V, H, N or D; X₄ is F, I, L, V, T, and S; X₅ is V, I, L, P, T or A; X₆ is V or I; X₇ is L or Q; X₈ is V or A; X₉ is E or V; X₁₀ is Q, P, L, V, A or E; X₁₁ is K or R; X₁₂ is E, D, K, N, Q or H; X₁₃ is H, P or R; X₁₄ is L, I, V, P, T, A, R, S or G; X₁₅ is T, I, N, F, S, Y, V, A or D; X₁₆ is V or I; X₁₇ is S, R, N, K, T, I or M; and X₁₈ is N, K, D, E, H or Q.

24. The decoy polypeptide of any one of claims 1 to 4, wherein the SIRPγ variant comprises an amino acid sequence set forth in any one of SEQ ID NOs: 3-14, 16-24, and 42.

25. The decoy polypeptide of any one of claims 1 to 4, wherein the SIRPγ variant comprises an amino acid sequence set forth in EEELQIIQPDKSVLVAAGETATLRCTITSLFPVGPIQWFRGAGPGRVLIYNQRDGPFPRV TTVSDGTKRNNMDFSIRISSITPADVGTYYCIKFRKGIPEDVEFKSGPGTXWH (SEQ ID NO: 15), wherein X is A, R, N, D, C, Q, E, G, H, I, L, K, M, F, P, S, T, W, Y, or V.

26. The decoy polypeptide of any one of claims 1 to 4, comprising the amino acid sequence of any one of SEQ ID NOs: 57-71 and 82-86 or an amino acid sequence that is at least about 90%, 91%, 92%, 93%, 94%, 95%, 96%, 97%, 98% or 99% identical to any one of SEQ ID NOs: 57-71, 74, and 82-86.

27. A decoy polypeptide comprising:
(a) a SIRPβP1 variant; and
(b) a human Fc variant comprising at least one amino acid substitution that reduces effector function compared to a wild type human Fc,
wherein the SIRPβ1 variant comprises at least one amino acid substitution relative to a wild type SIRPβ1, which substitution increases the affinity of the SIRPβ1 variant for CD47 as compared to the affinity of the wild type SIRPβ1 for CD47, and wherein the SIRPβ1 variant lacks a transmembrane domain.

28. The decoy polypeptide of claim 27, wherein the at least one amino acid substitution is within a d1 domain of the SIRPβ1 variant.

29. The decoy polypeptide of claim 27 or claim 28, wherein the amino acid sequence of the d1 domain of the SIRPβ1 variant is at least 90% identical to a sequence of a wild type SIRPβ1 d1 domain set forth in
(SEQ ID NO: 25)
EDELQVIQPEKSVSVAAGESATLRCAMTSLIPVGPIMWFRGAGAGRELIYN
QKEGHFPRVTTVSELTKRNNLDFSISISNITPADAGTYYCVKFRKGSPDDV
EFKSGAGTELSVRAKPS.

30. The decoy polypeptide of any one of claims 27 to 29, wherein the SIRPβ1 variant comprises one or more amino acid substitution at V6, M27, 131, M37, E47, K53, E54, H56, L66, N80, or V92, wherein the amino acid positions are relative to a wild-type human SIRPβ1 d1 domain sequence set forth in SEQ ID NO: 25.

31. The decoy polypeptide of claim 30, wherein the SIRPβ1 variant comprises the V6 substitution, and wherein the substitution is V6I.

32. The decoy polypeptide of claim 30 or claim 31, wherein the SIRPβ1 variant comprises the M27 substitution, and wherein the substitution is M27I.

33. The decoy polypeptide of any one of claims 30 to 32, wherein the SIRPβ1 variant comprises the I31 substitution, and wherein the substitution is I31F.

34. The decoy polypeptide of any one of claims 30 to 33, wherein the SIRPβ1 variant comprises the M37 substitution, and wherein the substitution is M37Q.

35. The decoy polypeptide of any one of claims 30 to 34, wherein the SIRPβ1 variant comprises the E47 substitution, and wherein the substitution is E47V.

36. The decoy polypeptide of any one of claims 30 to 35, wherein the SIRPβ1 variant comprises the K53 substitution, and wherein the substitution is K53R.

37. The decoy polypeptide of any one of claims 30 to 36, wherein the SIRPβ1 variant comprises the E54 substitution, and wherein the substitution is E54Q.

38. The decoy polypeptide of any one of claims 30 to 37, wherein the SIRPβ1 variant comprises the H56 substitution, and wherein the substitution is H56P.

39. The decoy polypeptide of any one of claims 30 to 38, wherein the SIRPβ1 variant comprises the L66 substitution, and wherein the substitution is L66T.

40. The decoy polypeptide of any one of claims 30 to 39, wherein the SIRPβ1 variant comprises the N80 substitution, and wherein the substitution is N80A, N80C, N80D, N80E, N80F, N80G, N80H, N80I, N80K, N80L, N80M, N80P, N80Q, N80R, N80S, N80T, N80V, N80W, or N80Y.

41. The decoy polypeptide of any one of claims 30 to 40, wherein the SIRPβ1 variant comprises the V92 substitution, and wherein the substitution is V92I.

42. The decoy polypeptide of any one of claims 27 to 29, wherein the SIRPβ1 variant comprises an amino acid sequence
(SEQ ID NO: 26)
EDELQIIQPEKSVSVAAGESATLRCAITSLFPVGPIQWFRGAGAGRVLIYN
QRQGPFPRVTTVSETTKRNNLDFSISISNITPADAGTYYCIKFRKGSPDDV
EFKSGAGTELSVRAKPS.

43. The decoy polypeptide of any one of claims 27-29, comprising an amino acid sequence of SEQ ID NO: 72 or an amino acid sequence that is at least about 90%, 91%, 92%, 93%, 94%, 95%, 96%, 97%, 98% or 99% identical to SEQ ID NO: 72.

44. The decoy polypeptide of any one of claims 27 to 29, wherein the SIRPβ1 variant comprises an amino acid sequence
(SEQ ID NO: 88)
EDELQIIQPEKSVSVAAGESATLRCAITSLFPVGPIQWFRGAGAGRVLIYN
QRQGPFPRVTTVSETTKRNNLDFSISISAITPADAGTYYCIKFRKGSPDDV
EFKSGAGTELSVRAKPS.

45. The decoy polypeptide of any one of claims 27-29, comprising an amino acid sequence of SEQ ID NO: 90 or an amino acid sequence that is at least about 90%, 91%, 92%, 93%, 94%, 95%, 96%, 97%, 98% or 99% identical to SEQ ID NO: 90.

46. A decoy polypeptide comprising:
(a) a SIRPβ2 variant; and
(b) a human Fc variant comprising at least one amino acid substitution that reduces effector function compared to a wild type human Fc,
wherein the SIRPβ2 variant comprises at least one amino acid substitution relative to a wild type SIRPβ2, which substitution increases the affinity of the SIRPβ2 variant for CD47 as compared to the affinity of the wild type SIRPβ2 for CD47, and wherein the SIRPβ2 variant lacks a transmembrane domain.

47. The decoy polypeptide of claim 46, wherein the at least one amino acid substitution is within a d1 domain of the SIRPβ2 variant.

48. The decoy polypeptide of claim 46 or claim 47, wherein the amino acid sequence of the d1 domain of the SIRPβ2 variant is at least 90% identical to a sequence of a wild type SIRPβ2 d1 domain set forth in
(SEQ ID NO: 27)
EEELQVIQPDKSISVAAGESATLHCTVTSLIPVGPIQWFRGAGPGRELIYN
QKEGHFPRVTTVSDLTKRNNMDFSIRISNITPADAGTYYCVKFRKGSPDHV
EFKSGAGTELSVRAKPS.

49. The decoy polypeptide of any one of claims 46 to 48, wherein the SIRPβ2 variant comprises one or more amino acid substitutions at V6, V27, 131, E47, K53, E54, H56, L66, N80, V92 or H101, wherein the amino acid positions are relative to a wild-type human SIRPβ2 d1 domain sequence set forth in SEQ ID NO: 27.

50. The decoy polypeptide of claim 49, wherein the SIRPβ2 variant comprises the V6 substitution, and wherein the substitution is V6I.

51. The decoy polypeptide of claim 49 or 50, wherein the SIRPβ2 variant comprises the V27 substitution, and wherein the substitution is V27I.

52. The decoy polypeptide of any one of claims 49 to 51, wherein the SIRPβ2 variant comprises the I31 substitution, and wherein the substitution is I31F.

53. The decoy polypeptide of any one of claims 49 to 52, wherein the SIRPβ2 variant comprises the E47 substitution, and wherein the substitution is E47V.

54. The decoy polypeptide of any one of claims 49 to 53, wherein the SIRPβ2 variant comprises the K53 substitution, and wherein the substitution is K53R.

55. The decoy polypeptide of any one of claims 49 to 54, wherein the SIRPβ2 variant comprises the E54 substitution, and wherein the substitution is E54Q.

56. The decoy polypeptide of any one of claims 49 to 55, wherein the SIRPβ2 variant comprises the H56 substitution, and wherein the substitution is H56P.

57. The decoy polypeptide of any one of claims 49 to 56, the SIRPβ2 variant comprises the L66 substitution, and wherein the substitution is L66T.

58. The decoy polypeptide of any one of claims 49 to 57, the SIRPβ2 variant comprises the N80 substitution, and wherein the substitution is N80A, N80C, N80D, N80E, N80F, N80G, N80H, N80I, N80K, N80L, N80M, N80P, N80Q, N80R, N80S, N80T, N80V, N80W, or N80Y.

59. The decoy polypeptide of any one of claims 49 to 58, the SIRPβ2 variant comprises the V92 substitution, and wherein the substitution is V92I.

60. The decoy polypeptide of any one of claims 49 to 59, the SIRPβ2 variant comprises the H101 substitution, and wherein the substitution is H101D.

61. The decoy polypeptide of any one of claims 46 to 48, wherein the SIRPβ2 variant comprises an amino acid sequence
(SEQ ID NO: 28)
EEELQIIQPDKSISVAAGESATLHCTITSLFPVGPIQWFRGAGPGRVLIYN
QRQGPFPRVTTVSDTTKRNNMDFSIRISNITPADAGTYYCIKFRKGSPDDV
EFKSGAGTELSVRAKPS.

62. The decoy polypeptide of any one of claims 46-48, comprising an amino acid sequence of SEQ ID NO: 73 or an amino acid sequence that is at least 90%, 91%, 92%, 93%, 94%, 95%, 96%, 97%, 98% or 99% identical to SEQ ID NO: 73.

63. The decoy polypeptide of any one of claims 46 to 48, wherein the SIRPβ2 variant comprises an amino acid sequence
(SEQ ID NO: 89)
EEELQIIQPDKSISVAAGESATLHCTITSLFPVGPIQWFRGAGPGRVLIYN
QRQGPFPRVTTVSDTTKRNNMDFSIRISAITPADAGTYYCIKFRKGSPDDV
EFKSGAGTELSVRAKPS.

64. The decoy polypeptide of any one of claims 46 to 48, comprising an amino acid sequence of SEQ ID NO: 91 or an amino acid sequence that is at least 90%, 91%, 92%, 93%, 94%, 95%, 96%, 97%, 98% or 99% identical to SEQ ID NO: 91.

65. The decoy polypeptide of any one of claims 1-64, wherein the human Fc variant comprises a modification that reduces glycosylation of the human Fc variant relative to a wild-type human Fc.

66. The decoy polypeptide of claim 65, wherein glycosylation is reduced by enzymatic deglycosylation, expression in a bacterial host, or modification of an amino acid residue required for glycosylation.

67. The decoy polypeptide of claim 65 or 66, wherein the modification that reduces glycosylation of the human Fc variant comprises a substitution at N297, wherein numbering is according to the EU index of Kabat.

68. The decoy polypeptide of claim 67, wherein the substitution at N297 is N297A, N297Q, N297D, N297H, N297G, or N297C, wherein numbering is according to the EU index of Kabat.

69. The decoy polypeptide of any one of claims 1-64, wherein the human Fc variant comprises substitutions at L234, L235, or G237 wherein numbering is according to the EU index of Kabat.

70. The decoy polypeptide of any one of claims 65-69, wherein the human Fc variant further comprises substitutions at L234, L235, and/or G237 wherein numbering is according to the EU index of Kabat.

71. The decoy polypeptide of claim 69 or 70, wherein the human Fc variant comprises L234A and L235A substitutions, wherein numbering is according to the EU index of Kabat.

72. The decoy polypeptide of claim 71, wherein the Fc variant further comprises a K322A substitution, wherein numbering is according to the EU index of Kabat.

73. The decoy polypeptide of any one of claims 1-25, 27-42, 44, 46-61, and 63, wherein the modification to the human Fc comprises E233P, L234V, L235A, delG236, A327G, A330S, and P331S mutations, wherein numbering is according to the EU index of Kabat.

74. The decoy polypeptide of any one of claims 1-64, wherein the human Fc variant is selected from the group consisting of: (a) a human IgG1 Fc comprising L234A, L235A, G237A, and N297A substitutions, wherein numbering is according to the EU index of Kabat; (b) a human IgG2 Fc comprising A330S, P331S, and N297A substitutions, wherein numbering is according to the EU index of Kabat; and (c) a human IgG4 Fc comprising S228P, E233P, F234V, L235A, delG236, and N297A mutations wherein numbering is according to the EU index of Kabat.

75. The decoy polypeptide of claim 74, wherein the human Fc variant is a human IgG1 Fc comprising L234A, L235A, G237A, and N297A substitutions wherein numbering is according to the EU index of Kabat.

76. The decoy polypeptide of claim 74, wherein the human Fc is a human IgG1 Fc and further comprises a D265A substitution, wherein numbering is according to the EU index of Kabat.

77. The decoy polypeptide of claim 75 or 76, wherein the human Fc variant exhibits ablated or reduced binding to an Fcγ receptor as compared to a wild-type human IgG1 Fc.

78. The decoy polypeptide of any one of claims 75 to 77, wherein the human Fc variant exhibits ablated or reduced binding to CD16a, CD32a, CD32b, CD32c, and CD64 Fcγ receptors as compared to a wild-type human IgG1 Fc.

79. The decoy polypeptide of any one of claims 75 to 78, wherein the human Fc variant exhibits ablated or reduced binding to C1q compared to a wild-type human IgG1 Fc.

80. The decoy polypeptide of claim 74, wherein the human Fc variant is a human IgG2 Fc comprising A330S, P331S, and N297A substitutions, wherein numbering is according to the EU index of Kabat.

81. The decoy polypeptide of claim 80, wherein the human Fc variant exhibits ablated or reduced binding to an Fcγ receptor as compared to a wild-type human IgG2 Fc.

82. The decoy polypeptide of claim 80 or 81, wherein the human Fc variant exhibits ablated or reduced binding to CD16a, CD32a, CD32b, CD32c, and CD64 Fcγ receptors as compared to a wild-type human IgG2 Fc.

83. The decoy polypeptide of any one of claims 80 to 82, wherein the human Fc variant exhibits ablated or reduced binding to C1q compared to a wild-type human IgG2 Fc.

84. The decoy polypeptide of claim 74, wherein the human Fc variant is a human IgG4 Fc comprising S228P, E233P, F234V, L235A, delG236, and N297A mutations, wherein numbering is according to the EU index of Kabat.

85. The decoy polypeptide of any one of claims 1-25, 27-42, 44, 46-61, and 63, wherein the human Fc variant is a human IgG4 Fc comprising an S228P substitution, wherein numbering is according to the EU index of Kabat.

86. The decoy polypeptide of any one of claims 1-25, 27-42, 44, 46-61, and 63, wherein the human Fc variant is a human IgG4 Fc comprising S228P and L235E substitutions, wherein numbering is according to the EU index of Kabat.

87. The decoy polypeptide of any one of claims 84 to 86, wherein the human Fc variant exhibits ablated or reduced binding to an Fcγ receptor as compared to a wild-type human IgG4 Fc.

88. The decoy polypeptide of any one of claims 84 to 87, wherein the human Fc variant exhibits ablated or reduced binding to CD16a and CD32b Fcγ receptors compared to the wild-type version of its human IgG4 Fc.

89. The decoy polypeptide of any one of claims 1-64, wherein the human Fc variant comprises an amino acid sequence set forth in any one of SEQ ID NOs: 48-51, 53-56, 93-96, and 98-101.

90. The decoy polypeptide of any one of claims 1 to 89, wherein the human Fc variant binds to an Fcγ receptor with a K_{D} greater than about 5×10⁻⁶ M.

91. The decoy polypeptide of any one of claims 1 to 90, which does not cause acute anemia in rodents and non-human primates following administration.

92. The decoy polypeptide of any one of claims 1 to 91, which does not cause acute anemia in humans following administration.

93. The decoy polypeptide of any one of claims 1 to 92, which blocks binding of CD47 to a ligand.

94. The decoy polypeptide of claim 93, wherein the ligand is SIRPα or SIRPγ.

95. The decoy polypeptide of any one of claims 1 to 94, wherein the decoy polypeptide binds to CD47 expressed on the surface of a cell.

96. The decoy polypeptide of claim 95, wherein the cell is a tumor cell, virally infected cell, bacterially infected cell, damaged red blood cell, arterial plaque cell, fibrotic tissue cell, a healthy normal cell such as hematopoietic stem cell.

97. The decoy polypeptide of any one of claim 95 or 96, wherein the binding of the polypeptide to CD47 expressed on the surface of the cell induces or enhances phagocytosis or ADCC of the cell.

98. The decoy polypeptide of any one of claims 1 to 97, wherein the decoy polypeptide is a dimer.

99. The decoy polypeptide of claim 98, wherein the dimer is a homodimer.

100. The decoy polypeptide of any one of claims 1 to 99, further comprising a detectable label.

101. A composition comprising the decoy polypeptide of any one of claims 1 to 100 and a pharmaceutically acceptable excipient.

102. The composition of claim 101, further comprising one or more additional agents.

103. The composition of claim 102, wherein the one or more additional agents is a chemotherapeutic agent, a kinase inhibitor, a proteasome inhibitor, an inhibitor of a viral DNA polymerase, an inhibitor of a viral RNA polymerase, or a therapeutic antibody.

104. The composition of claim 103, wherein the one or more additional agents is a therapeutic antibody.

105. The composition of claim 104, wherein the therapeutic antibody is cetuximab, necitumumab, pembrolizumab, nivolumab, pidilizumab, ipilimumab, tremelimumab, urelumab, daratumumab, trastuzumab, trastuzumab emtansine, pertuzumab, elotuzumab, rituximab, ofatumumab, obinutuzumab, panitumumab, brentuximab vedotin, MSB0010718C, belimumab, bevacizumab, denosumab, ramucirumab, or atezolizumab.

106. The composition of claim 104, wherein the therapeutic antibody targets a HLA/peptide or MHC/peptide complex comprising a peptide derived from NY-ESO-1/LAGE1, SSX-2, a member of the MAGE protein family, gp100/pmel17, MelanA/MART1, gp75/TRP1, tyrosinase, TRP2, CEA, PSA, TAG-72, Immature laminin receptor, MOK/RAGE-1, WT-1, Her2/neu, EphA3, SAP-1, BING-4, Ep-CAM, MUC1, PRAME, survivin, Mesothelin, BRCA1, BRCA2, CDK4, CML66, MART-2, p53, Ras, β-catenin, TGF-βRII, HPV E6, or HPV E7.

107. The composition of claim 104, wherein the therapeutic antibody binds an antigen on a cancer cell, an immune cell, a pathogen-infected cell, or a hematopoietic stem cell.

108. The composition of claim 107, wherein the therapeutic antibody binds an antigen on a cancer cell, and wherein the antigen is EGFR, Her2/neu, CD19, CD20, CD22, CD25, CD30, CD33, CD38, CD45, CD47, CD56, CD70, CD117, or EpCAM.

109. The composition of claim 108, wherein the therapeutic antibody binds an antigen on an immune cell, and wherein the antigen is M1prime, CD2, CD3, CD4, CD5, CD8, CD19, CD20, CD22, CD25, CD38, CD56, PD-1, PD-L1, CTLA4, BTLA, TIM3, LAG3, OX40, GITR or CD137 (4-1BB).

110. The composition of claim 107, wherein the therapeutic antibody binds an antigen on a pathogen-infected cell, and wherein the antigen is a CMV protein, UL18, UL11, pp65, gB, pp150, an HIV envelope protein, Gp41, Gp120, V1V2 glycan, V3 glycan, and influenza hemagglutinin.

111. The composition of claim 107, wherein the therapeutic antibody binds an antigen on a hematopoietic stem cell, and wherein the antigen is CD11, CD45, CD117 or Sca1.

112. An isolated nucleic acid encoding the decoy polypeptide of any one of claims 1 to 99.

113. A vector comprising the nucleic acid of claim 112.

114. A host cell comprising the nucleic acid of claim 112 or the vector of claim 113.

115. A method of producing a decoy polypeptide, comprising culturing the host cell of claim 114 under conditions where the decoy polypeptide is expressed and recovering the decoy polypeptide.

116. A method of modulating phagocytosis or ADCC of a cell expressing CD47, the method comprising contacting the cell with the decoy polypeptide of any one of claims 1 to 99 or the composition of any one of claims 101 to 111.

117. A method of treating a subject having a disease or disorder, comprising administering an effective amount of the decoy polypeptide of any one of claims 1 to 99 or the composition of any one of claims 101 to 111 to the subject.

118. The method of claim 117, wherein the disease or disorder is cancer, anemia, a viral infection, a bacterial infection, an autoimmune disease or an inflammatory disorder, asthma, an allergy, a transplant rejection, atherosclerosis, or fibrosis.

119. The method of claim 118, wherein the disease or disorder is cancer, and wherein the cancer is cancer is solid tumor, hematological cancer, acute myeloid leukemia, chronic lymphocytic leukemia, chronic myeloid leukemia, acute lymphoblastic leukemia, non-Hodgkin lymphoma, Hodgkin lymphoma, multiple myeloma, bladder cancer, pancreatic cancer, cervical cancer, endometrial cancer, lung cancer, bronchus cancer, liver cancer, ovarian cancer, colon and rectal cancer, stomach cancer, gastric cancer, gallbladder cancer, gastrointestinal stromal tumor cancer, thyroid cancer, head and neck cancer, oropharyngeal cancer, esophageal cancer, melanoma, non-melanoma skin cancer, Merkel cell carcinoma, virally induced cancer, neuroblastoma, breast cancer, prostate cancer, renal cancer, renal cell cancer, renal pelvis cancer, leukemia, lymphoma, sarcoma, glioma, brain tumor, and carcinoma.

120. The method of claim 118, wherein the disease or disorder is an autoimmune disease or inflammatory disorder, and wherein the autoimmune disease or inflammatory disorder is multiple sclerosis, rheumatoid arthritis, a spondyloarthropathy, systemic lupus erythematosus, an antibody-mediated inflammatory or autoimmune disease, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, and inflammatory autoimmune myositis.

121. The decoy polypeptide of any one of claims 1 to 99 or the composition of any one of claims 101-111 for use in treating cancer, viral infection, bacterial infection, auto-immune disease, asthma, allergy, transplant rejection, atherosclerosis, or fibrosis.

122. The decoy polypeptide of any one of claims 1 to 99 or the composition of any one of claims 101 to 111 for use in preconditioning for a hematopoietic stem cell transplant.

123. A method of detecting a CD47⁺ cell in a population of cells comprising contacting the population of cells with the decoy polypeptide of claim 100, and detecting binding of the decoy polypeptide to CD47⁺ cells, wherein the detecting of the binding indicates the presence of CD47⁺ cells.

124. The method of claim 123, wherein the CD47⁺ cell is a tumor cell, a virally infected cell, a bacterially infected cell, an autoreactive T or B cell, a damaged red blood cell, an arterial plaque cell, or a fibrotic tissue cell.

125. The method of claim 123 or claim 124, wherein the contacting is in vivo.

126. The method of claim 123 or claim 124, wherein the contacting is in vitro.

127. A method of purifying a CD47⁺ cell from a population of cells, the method comprising contacting a population of cells with the decoy polypeptide of any one of claims 1-100 and isolating the cells bound to the decoy polypeptide.

128. A chimeric molecule comprising the decoy polypeptide of any one of claims 1 to 99 and an immune checkpoint inhibitor, a co-stimulatory molecule, a cytokine, or an attenuated cytokine.

129. The chimeric molecule of claim 128, wherein the decoy polypeptide is linked to the immune checkpoint inhibitor, co-stimulatory molecule, cytokine, or attenuated cytokine through a linker sequence.

130. The chimeric molecule of claim 129, wherein the linker sequence comprises Gly and Ser.

131. The chimeric molecule of claim 130, wherein the linker sequence comprises GGGGSGGGGS (SEQ ID NO: 29).

132. The chimeric molecule of any one of claims 128 to 131, wherein the decoy polypeptide is fused to the N-terminal or C-terminal end of the immune checkpoint inhibitor, co-stimulatory molecule, cytokine, or attenuated cytokine.

133. The chimeric molecule of claim any one of claims 128 to 132, wherein the decoy polypeptide is fused to an immune checkpoint inhibitor, and wherein the immune checkpoint inhibitor comprises a sequence of a PD-1 or PD-L1 antagonist, a BTLA or CD160 antagonist, a phosphatidylserine antagonist, MFGE8, TIM1, TIM3, or TIM4.

134. The chimeric molecule of claim any one of claims 128 to 132, wherein the decoy polypeptide is fused to a co-stimulatory molecule, and wherein the co-stimulatory molecule comprises a sequence of a CD40 agonist, a 41BBL or CD137 agonist.

135. The chimeric molecule of claim any one of claims 128 to 132, wherein the decoy polypeptide is fused to a cytokine, and wherein the cytokine comprises a sequence of an IL2.

136. The chimeric molecule of 135, wherein the IL2 sequence comprises mutations D20T and F42A.

137. The chimeric molecule of claim any one of claims 128 to 132, wherein the decoy polypeptide is fused to a cytokine polypeptide, and wherein the cytokine is attenuated.

138. The chimeric molecule of any one of claims 128 to 133, comprising an amino acid sequence set forth in SEQ ID NO: 30 or SEQ ID NO: 102.

139. The chimeric molecule of any one of claims 128 to 133, comprising an amino acid sequence set forth in SEQ ID NO: 31 or SEQ ID NO: 103.

140. The chimeric molecule of any one of claims 128 to 133, comprising an amino acid sequence set forth in any one of SEQ ID NOs: 32-39 or 104-111.
